(19) United States
(12) Patent Application Publication
(10) Pub. No.: US 2008/0281226 A1
(43) Pub. Date: Nov. 13, 2008

(54) Core Biopsy Device with Specimen Length Adjustment

(75) Inventor: Stephen F. Peters, Hickory Corners, MI (US)

Correspondence Address: MCGARRY BAIR PC, 32 Market Ave. SW, SUITE 500, GRAND RAPIDS, MI, 49503, US

(73) Assignee: INRAD, INC., Grand Rapids, MI (US)

(21) Appl. No.: 12/182,463

(22) Filed: Jul. 30, 2008

Related U.S. Application Data
(63) Continuation-in-part of application No. 10/908,427, filed on May 11, 2005.
(60) Provisional application No. 60/521,518, filed on May 11, 2004.

(51) Int. Cl. A61B 10/02

(52) U.S. Cl. 600/567

(57) ABSTRACT

A biopsy device comprises a housing carrying a stylet is telescoping register with a coring cannula and a specimen length adjuster operably coupled to the stylet to move the stylet relative to the coring cannula to set the size of the biopsy sample to be collected. The coring cannula is moved over the stylet and rotated relative to the stylet to excise the biopsy sample from surrounding tissue. A trigger assembly is provided that includes a release button on opposing sides of the housing as well as a rear trigger button that enables a user to comfortably actuate the biopsy device using any of the three trigger buttons.

### CROSS-REFERENCE TO RELATED APPLICATION

This application is a continuation-in-part of U.S. application Ser. No. 10/908,427, filed May 11, 2005, which claims the benefit of U.S. provisional application No. 60/521,518, filed May 11, 2004, both of which are incorporated herein in their entirety.

### BACKGROUND OF THE INVENTION

It is frequently necessary to sample or remove a sample from a suspect tissue for testing. In humans, such a sample removal is particularly useful in the diagnosis and treatment of cancerous or pre-cancerous conditions. In the case of suspected cancer, particularly cancer of the breast, early detection and diagnosis is critical to the success of the patient's treatment and recovery.

Various techniques are available to aid in detection and diagnosis, including physical examination and imaging, such as mammography, x-ray, ultrasound, magnetic resonance imaging (MRI), and the like. When a condition is detected that suggests the possibility of cancer, a biopsy can be performed to obtain tissue samples for a complete diagnosis.

One biopsy technique frequently performed is a core biopsy, which uses a core biopsy device in which a cannula is inserted into the tissue of interest, thereby coring a biopsy sample from the tissue having a cross section similar to that of the cannula, and which is retained within the cannula. The cannula, with the biopsy sample, is then removed from the tissue, followed by cytological and/or histological analysis of the sample.

One group of core biopsy devices is based on the combination of a notched inner stylet and an outer severing cannula. The stylet is retained within the lumen of the outer cannula such that the pointed end of the stylet closes off the open end of the cannula. The stylet and cannula are advanced into the tissue mass until they are near the desired biopsy site. The stylet is then advanced relative to the outer cannula to expose the notch to the biopsy site where the tissue prolapses into the notch. The outer cannula is then advanced to sever the tissue in the notch. The disadvantage of this method is that it produces a small core biopsy relative to the outer cannula size since the cross section of the sample is substantially equal to the cross section of the stylet notch, which is substantially smaller than the cross section of the outer cannula. The advantage of this method is that the sample is completely severed from the tissue mass and securely retained within the notch.

Another group of core biopsy devices is based on a coring cannula in combination with a non-notched stylet. The stylet is used to plug the end of the coring cannula during the insertion of the coring cannula into the tissue adjacent the biopsy site. The coring cannula is then advanced relative to the stylet into the biopsy site to retain a sample within the coring cannula. The advantage of this device is that a full core biopsy sample is obtained. That is, the cross section of the sample is substantially equal to the cross section of the coring cannula. The full core sample provides a much larger sample which is highly advantageous.

The disadvantage of this full core device is that the end of the sample is not positively severed from the tissue mass, creating the possibility that the biopsy sample will be pulled out of the coring cannula upon the withdrawal of the coring cannula. This can happen if the forces holding the sample in the coring cannula are not sufficient to tear the end of the sample from the tissue mass. Since the sample normally comprises wetted tissue that completely fills the coring cannula, the suction force and/or the frictional force between the tissue sample and the inner wall of the coring cannula are the dominate forces for retaining the sample in the cannula. However, if these forces are not sufficient to tear the end of the sample from the tissue mass, the sample will be pulled out of the coring cannula upon the removal of the coring cannula. Some practitioners pivot the biopsy device in hopes that the end of the cannula will at least partially sever the attached portion of the sample. However, this is not preferred as it increases the damage to the remaining tissue.

Attempts have been made to improve the severing of the sample from the tissue mass for the full core device. In some cases, the interior of the coring cannula is provided with a raceway in which a severing finger could be advanced/retracted. After the advancing of the cannula to take the core sample, the severing finger is advanced, guided by the raceway, to sever the tissue. In some cases, when the finger is advanced, it closes the end of the coring cannula and is left in the advanced position during removal. A disadvantage of this method is that the sample is not truly a full core sample since part of the interior of the cannula was reserved for the raceway. If the sample produced by the cannula with the raceway was the same size as the full core sample, the cannula with the raceway would require a larger cross sectional cannula, which is not desirable. In the biopsy art, it is highly desirable to minimize the cross section of the cannula to minimize the damage to the surrounding tissue and to minimize the invasiveness of the procedure. Generally, the smaller the cross section, the less pain the patient experiences after the procedure, and the more desirable the device.

Another alternative to severing the sample end in a full core device comprises the addition of a cutting cannula that circumscribes the coring cannula. The cutting cannula has a cutting element that severs the sample within the coring cannula or at the tip of the coring cannula. For example, the device in U.S. Pat. No. 5,755,642 discloses fingers that are deflected through windows in the coring cannula into the interior of the coring cannula to sever the tissue. The internally severing devices have the disadvantage in that the resulting sample is shorter in length than the amount of tissue that is received within the interior since the finger enters the coring cannula proximal to the coring cannula tip. Since not all of the sample received within the coring cannula is severed, extra care must be taken to ensure that the lesion or other relevant portion of the tissue to be biopsied lies behind the end location of the severing finger. This is made more difficult in that the practitioner cannot use the end of the cannula for marking the extent of the biopsy specimen.

U.S. Pat. No. 6,651,254 discloses another approach using a tubular cutting member slidably mounted to the coring cannula, with the tubular cutting member having a spring memory finger that deflects downwardly over the coring cannula tip to close off the coring cannula opening and sever the sample from the tissue mass. The finger has a sharpened edge to affect the cutting. The disadvantage of this structure is that the coring and cutting cannulae are made from multiple pieces and include built in stops, which increase the assembly requirements and cost of the device. Also, the cutting finger needs to be of sufficient width to span the coring cannula opening to ensure the complete severing of the end of the specimen.

Another disadvantage of all of the full core devices is that they rely on the relative movement between the coring cannula and the stylet to expel the sample from the interior of the coring cannula. The use of the stylet to force out the sample can damage the sample. The damage can be great enough to render the sample unsuitable for testing. This can be very detrimental since some lesions being sampled are small enough that the entire lesion is contained within the sample. For larger lesions, some practitioners will take multiple samples to allow for potential damage of one of the samples. This practice increases the invasiveness of the procedure and the pain to the patient.

While there have been many attempts in the art to produce a workable core biopsy device, there is still a strong need for a core biopsy device that minimizes patient discomfort, insures a complete excision of the biopsy sample from the surrounding tissue, enables the biopsy sample to be removed from the device without disturbance of the sample, and is simple and cost effective to manufacture.

### SUMMARY OF THE INVENTION

In one embodiment, the invention relates to a biopsy device for the percutaneous removal of a specimen from a tissue mass. The biopsy device comprises a stylet having a distal tip, a coring cannula defining a lumen for receiving the stylet and having an excising finger at a distal end of the coring cannula and operable between an inserting position and an excising position, where the distal tip of the stylet is spaced from the distal end of the coring cannula a predetermined distance, an actuator operably coupled to the coring cannula to move the coring cannula relative to the stylet from the inserting position to the excising position and to rotate the coring cannula in the excising position, and a specimen length adjuster operably coupled to the stylet to move the stylet relative to the coring cannula to set the predetermined distance.

In another embodiment, the invention relates to a method of conducting a percutaneous biopsy by removing a core specimen from a predetermined site in a tissue mass with a biopsy device comprising an axially movable coring cannula defining a lumen, and a stylet received within the lumen. The method comprises setting the length of the core specimen by adjusting the relative positions of the stylet and the coring cannula, partially forming a core specimen by moving the coring cannula relative to the stylet, and severing the core specimen from the tissue mass by rotating the coring cannula.

In yet another embodiment, the invention relates to a method of operating a biopsy device comprising an axially movable coring cannula defining a lumen, and a stylet received within the lumen. The method comprises axially moving the coring cannula relative to the stylet from a ready position to a cutting position to form a tissue chamber between a distal end of the stylet and a distal end of the coring cannula, rotating the coring cannula in the cutting position, and adjusting the relative positions of the stylet and the coring cannula to place the biopsy device in the ready position and to define the length of the tissue chamber.

In still another embodiment, the invention relates to a biopsy device for the percutaneous removal of a specimen from a tissue mass. The biopsy device comprises a housing having opposing sides, a needle assembly carried by the housing and comprising a stylet having a distal tip and a cannula defining a lumen for receiving the stylet, a firing assembly carried by the housing and operably coupled to the needle assembly to effect the relative movement of the cannula and the stylet to obtain a specimen, and a trigger assembly operably coupled to the firing assembly to control the actuation of the firing assembly and having a release button on each opposing side of the housing, wherein pressing either release button effects the actuation of the firing assembly.

### BRIEF DESCRIPTION OF THE DRAWINGS

In the drawings:

FIG. 1 is a perspective view of a lesion within a tissue mass and a first embodiment of a core biopsy device comprising an actuator assembly and a cannula assembly according to the invention for obtaining a core biopsy sample from the lesion.

FIG. 2 is a perspective view of a cannula assembly comprising a coring cannula, a spoon cannula, and a stylet, with the coring cannula in an excising position.

FIG. 3 is an exploded view of the coring cannula, spoon cannula, and stylet comprising the cannula assembly illustrated in FIG. 2.

FIG. 4 is an enlarged perspective view of a distal end of the stylet illustrated in FIG. 3.

FIG. 5 is an enlarged perspective view of a distal end of the spoon cannula illustrated in FIG. 3.

FIG. 6 is an enlarged perspective view of a distal end of the coring cannula illustrated in FIG. 3.

FIG. 7 is an enlarged view of a distal end of the coring cannula and the spoon cannula telescopically received therein, with the coring cannula in the excising position.

FIG. 7A is a sectional view taken along view line 7A-7A of FIG. 7.

FIG. 8A is a longitudinal sectional view illustrating the cannula assembly in a “cocked” configuration ready for insertion into the tissue mass.

FIG. 8B is a longitudinal sectional view illustrating the cannula assembly in a sampling configuration with the coring cannula and the spoon cannula projected distally over the stylet.

FIG. 8C is a longitudinal sectional view illustrating the cannula assembly in a sample excising configuration with the coring cannula extending distally of the spoon cannula.

FIG. 8D is a longitudinal sectional view illustrating the cannula assembly in an alternative “cocked” configuration ready for insertion into the tissue mass.

FIGS. 9A-G are longitudinal sectional views of the cannula assembly illustrated in FIG. 2 at various steps in the process of obtaining a core biopsy sample.

FIG. 10 is an enlarged view of a distal end of a second embodiment of the core biopsy device.

FIG. 11 is an enlarged view of a distal end of a third embodiment of the core biopsy device illustrating the spoon cannula and a coring cannula having a spoon section.

FIG. 11A is a sectional view similar to that taken along view line 11A-11A illustrating the spoon cannula supporting a core biopsy sample and the coring cannula rotated to diametrically juxtapose the cannulae spoon sections to enclose the core biopsy sample.

FIG. 12 is an enlarged view of a distal end of a fourth embodiment of the core biopsy device.

FIG. 12A is an enlarged perspective view of a distal end of the spoon cannula illustrated in FIG. 5 having a scalloped excising tip.

FIG. 13 is a cutaway perspective view of a first embodiment of an actuator assembly for controlling the operation of the cannula assembly for obtaining a core biopsy sample from the lesion, taken along view line 13-13 of FIG. 1.

FIG. 14 is an exploded view of the actuator assembly illustrated in FIG. 13.

FIG. 15 is an enlarged exploded view of the actuator assembly illustrated in FIG. 14.

FIGS. 16A-D are alternate views of a retraction body comprising a portion of the actuator assembly illustrated in FIG. 13.

FIGS. 17A-F are alternate views of a driving sleeve comprising a portion of the actuator assembly illustrated in FIG. 13.

FIG. 18 is an elevation view of the driving sleeve and a rotating cylinder comprising a portion of the actuator assembly of FIG. 13 with portions in phantom to illustrate the operable engagement of the driving sleeve with the rotating cylinder.

FIG. 19 is an enlarged cutaway perspective view of the actuator assembly illustrated in FIG. 13.

FIG. 20 is a cutaway perspective view of the actuator assembly illustrated in FIG. 13 in a first, cocked position.

FIG. 21 is a cutaway side elevation view of the actuator assembly illustrated in FIG. 20.

FIG. 22 is a cutaway perspective view of the actuator assembly illustrated in FIG. 13 in a second, fired position.

FIG. 23 is a cutaway side elevation view of the actuator assembly illustrated in FIG. 22.

FIG. 24 is a perspective view of a second embodiment of a core biopsy device comprising a second embodiment of an actuator assembly and a cannula assembly according to the invention.

FIG. 25 is an exploded view of the core biopsy device illustrated in FIG. 24, illustrating a cocking handle assembly, a shuttle assembly, a trigger assembly, a sample size control assembly, and a cannula operation assembly.

FIGS. 26A-C are perspective and enlarged partial views of a right housing shell comprising an element of the core biopsy device illustrated in FIG. 24.

FIG. 27 is an enlarged view of a portion of the right housing shell illustrated in FIGS. 26A-C.

FIGS. 28A-C are perspective and enlarged partial views of a left housing shell comprising an element of the core biopsy device illustrated in FIG. 24.

FIG. 29 is an enlarged view of a portion of the right housing shell illustrated in FIGS. 26A-C.

FIG. 30 is a perspective view of the underside of a handle comprising an element of the cocking handle assembly illustrated in FIG. 24.

FIG. 31 is a perspective view of a spring comprising an element of the cocking handle assembly illustrated in FIG. 24.

FIG. 32 is a perspective view of a swing arm comprising an element of the cocking handle assembly illustrated in FIG. 24.

FIGS. 33A-C are perspective views of a latch comprising an element of the cocking handle assembly illustrated in FIG. 24.

FIGS. 34A-B are perspective views of a button comprising an element of the cocking handle assembly and trigger assembly illustrated in FIG. 24.

FIGS. 35A-G are perspective and enlarged partial views of a shuttle comprising an element of the shuttle assembly illustrated in FIG. 24.

FIG. 36 is a perspective view of a spring retainer comprising an element of the shuttle assembly illustrated in FIG. 24.

FIG. 37 is a perspective view of a cam spring comprising an element of the shuttle assembly illustrated in FIG. 24.

FIGS. 38A-B are perspective views of a cam block comprising an element of the shuttle assembly illustrated in FIG. 24.

FIGS. 39A-D are perspective and enlarged partial views of a firing cage comprising an element of the trigger assembly illustrated in FIG. 24.

FIGS. 40A-C are perspective and enlarged partial views of a nosepiece comprising an element of the sample size control assembly illustrated in FIG. 24.

FIGS. 41A-B are perspective views of an adjustment member comprising an element of the sample size control assembly illustrated in FIG. 24.

FIG. 42 is a perspective view of the sample size control assembly illustrated in FIG. 24.

FIGS. 43A-B are perspective views of a latch plate comprising an element of the cannula operation assembly illustrated in FIG. 24.

FIGS. 44A-C are perspective views of a spoon cannula carriage comprising an element of the cannula operation assembly illustrated in FIG. 24.

FIG. 45 is a perspective view of the spoon cannula carriage of FIGS. 44A-C with the spoon cannula affixed thereto.

FIGS. 46A-D are perspective and enlarged partial views of a cutting cannula carriage comprising an element of the cannula operation assembly illustrated in FIG. 24.

FIGS. 47A-C are perspective views of a helical drive member comprising an element of the cannula operation assembly illustrated in FIG. 24.

FIGS. 48A-B are perspective views of a rotating driven member comprising an element of the cannula operation assembly illustrated in FIG. 24.

FIG. 49 is a left elevational view of the core biopsy device illustrated in FIG. 24 illustrating the core biopsy device in an uncocked and fired configuration, with elements removed for clarity.

FIG. 49A is a partial cutaway perspective view of a portion of the core biopsy device illustrated in FIG. 24 illustrating the cam block in a first position during cocking of the device.

FIG. 49B is a partial cutaway plan view of a portion of the core biopsy device illustrated in FIG. 24 illustrating the cam block in a first position during cocking of the device, and in a second position during firing of the device, the second position being shown in phantom.

FIG. 50 is a left elevational view of the core biopsy device illustrated in FIG. 24 illustrating the core biopsy device in a first configuration ready for cocking, with elements removed for clarity.

FIG. 51 is a left elevational view of the core biopsy device illustrated in FIG. 24 illustrating the core biopsy device in a first cocked configuration, with elements removed for clarity.

FIG. 52 is a left elevational view of the core biopsy device illustrated in FIG. 24 illustrating the core biopsy device in a second configuration ready for cocking, with elements removed for clarity.

FIG. 53 is a left elevational view of the core biopsy device illustrated in FIG. 24 illustrating the core biopsy device being placed in a second cocked configuration, with the elements removed for clarity.

FIG. 54 is a left elevational view of the core biopsy device illustrated in FIG. 24 illustrating the core biopsy device in an initial fired position with the spoon cannula and the coring cannula being advanced for introduction into a lesion.

FIG. 55 is a left elevational view of the core biopsy device illustrated in FIG. 24 illustrating the core biopsy device in an intermediate fired position with the coring cannula being advanced beyond the spoon cannula for excision of a biopsy sample.

FIG. 56 is a left elevational view of the core biopsy device illustrated in FIG. 24 illustrating the core biopsy device in a final fired positioned with the coring cannula being rotated for excision of a biopsy sample.

FIG. 57 is a perspective view of a third embodiment of a core biopsy device comprising a third embodiment of an actuator assembly and a cannula assembly according to the invention.

FIG. 58 is an exploded view of the core biopsy device illustrated in FIG. 57, illustrating cocking handle assembly, a trigger assembly, a cannula operation assembly and a sample size control assembly.

FIG. 59 is a perspective view of a right housing shell comprising an element of the core biopsy device illustrated in FIG. 57.

FIG. 60 is a perspective view of the underside of a cocking lever comprising an element of the cocking handle assembly illustrated in FIG. 58.

FIG. 61A is a perspective view of a swing arm comprising an element of the cocking handle assembly illustrated in FIG. 58.

FIG. 61B is a perspective view of a spring comprising an element of the cocking handle assembly illustrated in FIG. 58.

FIG. 62 is a perspective view of a latch and latch buttons comprising elements of the cocking handle assembly illustrated in FIG. 58.

FIG. 63A-B are perspective views of the assembled cocking handle assembly.

FIG. 64A-B are perspective views of a shuttle comprising an element of the trigger assembly illustrated in FIG. 58.

FIG. 65 is a perspective view of a spring retainer comprising an element of the trigger assembly illustrated in FIG. 58.

FIG. 66A-B are perspective views of a toggle piece comprising an element of the trigger assembly illustrated in FIG. 58.

FIG. 67 is a perspective view of a toggle button comprising an element of the trigger assembly illustrated in FIG. 58.

FIG. 68 is a perspective view of the assembled trigger assembly.

FIGS. 69A-B are perspective views of a spoon cannula carriage comprising an element of the cannula operation assembly illustrated in FIG. 58.

FIG. 70A-B are perspective views of a coring cannula carriage comprising an element of the cannula operation assembly illustrated in FIG. 58.

FIG. 71 is a perspective view of a helical drive member comprising an element of the cannula operation assembly illustrated in FIG. 58.

FIG. 72 is a perspective view of a rotating driven member comprising an element of the cannula operation assembly illustrated in FIG. 58.

FIG. 73 is a perspective view of the assembled cannula operation assembly.

FIG. 74 is a perspective view of a stylet carriage comprising an element of the sample size control assembly illustrated in FIG. 58.

FIG. 75 is a side view of an adjustment member comprising an element of the sample size control assembly illustrated in FIG. 58.

FIG. 76 is a side perspective view of the assembled sample size control assembly.

FIG. 77 is an elevational view of the core biopsy device illustrated in FIG. 57 illustrating the device in an uncocked and fired configuration, with elements removed for clarity.

FIG. 78 is a view similar to FIG. 77, illustrating the core biopsy device in a first configuration ready for a first cocking.

FIG. 79 is a view similar to FIG. 77, illustrating the core biopsy device in a first cocked configuration and in a second configuration ready for a second cocking.

FIG. 80 illustrates the relative movement of the shuttle and the toggle piece during operation of the core biopsy device.

FIG. 81 is a view similar to FIG. 77, illustrating the core biopsy device being placed in a second cocked configuration

FIG. 82A is a side view of the sample size control assembly, illustrating the specimen size to be collected by the core biopsy device being selected.

FIG. 82B is a longitudinal sectional view illustrating the cannula assembly in the cocked configuration ready for insertion into the tissue mass and the movement of the stylet for the selection of the specimen size to be collected.

FIG. 82C is a longitudinal sectional view related to FIG. 82C, illustrating the cannula assembly in a sample excising configuration and the corresponding specimen size to be collected.

FIG. 83 illustrates the core biopsy device being actuated or fired.

FIG. 84 is a view similar to FIG. 77, illustrating the core biopsy device in an initial fired position with the spoon cannula and the coring cannula being advanced for introduction into a lesion.

FIG. 85 is a view similar to FIG. 77, illustrating the core biopsy device in an intermediate fired position with the coring cannula being advanced beyond the spoon cannula for excision of a biopsy sample.

FIG. 86 is a view similar to FIG. 77, illustrating the core biopsy device in a final fired positioned with the coring cannula being rotated for excision of a biopsy sample.

### DESCRIPTION OF AN EMBODIMENT OF THE INVENTION

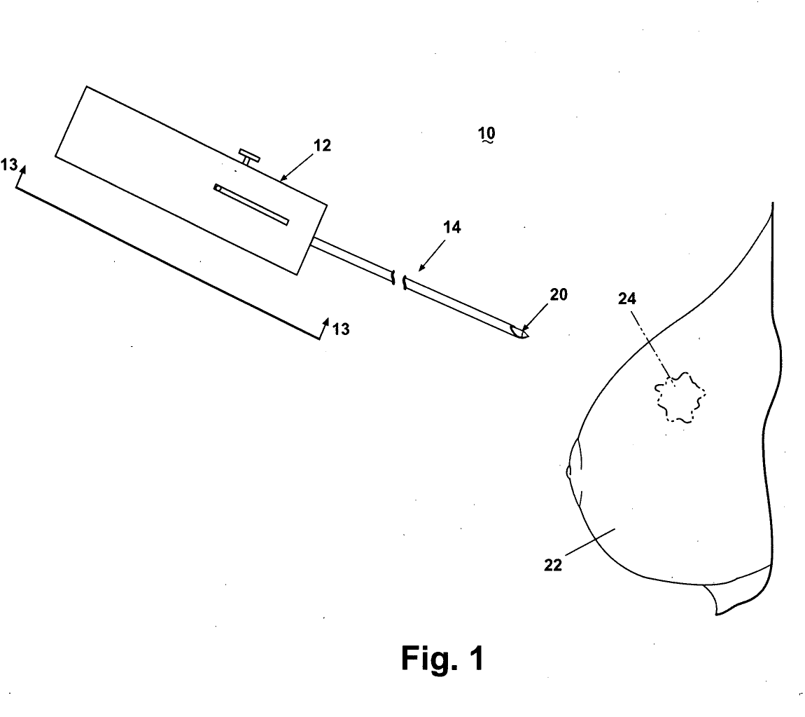

Referring now to the drawings, and specifically to FIG. 1, a core biopsy device 10 is illustrated comprising an actuator assembly 12 structurally and operably connected to a cannula assembly 14. The cannula assembly 14 is utilized to penetrate a tissue mass 22 for obtaining a core biopsy sample from a lesion 24 as more specifically described hereinafter. An embodiment of the actuator assembly 12 is described and illustrated herein comprising an automated, integrated hand-held device capable of controlling the acquisition and removal of the core biopsy sample from the lesion 24. An actuator assembly 12 is preferably utilized that is capable of automated firing of the cannula assembly 14, with the additional capability of firing a pair of telescoping cannulae and a stylet with one triggering action, or firing an inner cannula and an outer cannula independently. As described and illustrated herein, the actuator assembly 12 is capable of controlled rotation of the outer cannula around the inner cannula after the cannulae have been fired to excise the core biopsy sample from the surrounding lesion 24.

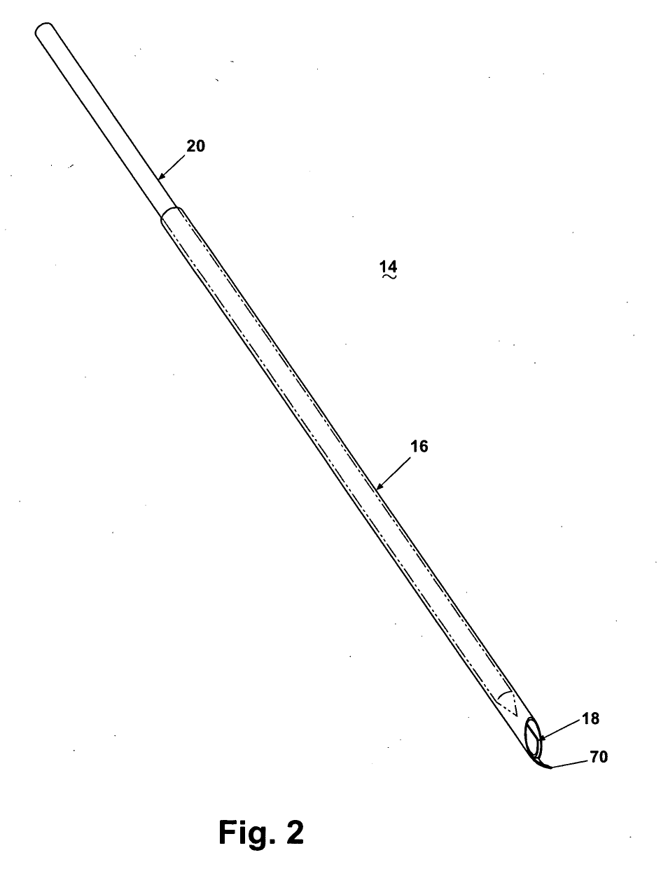

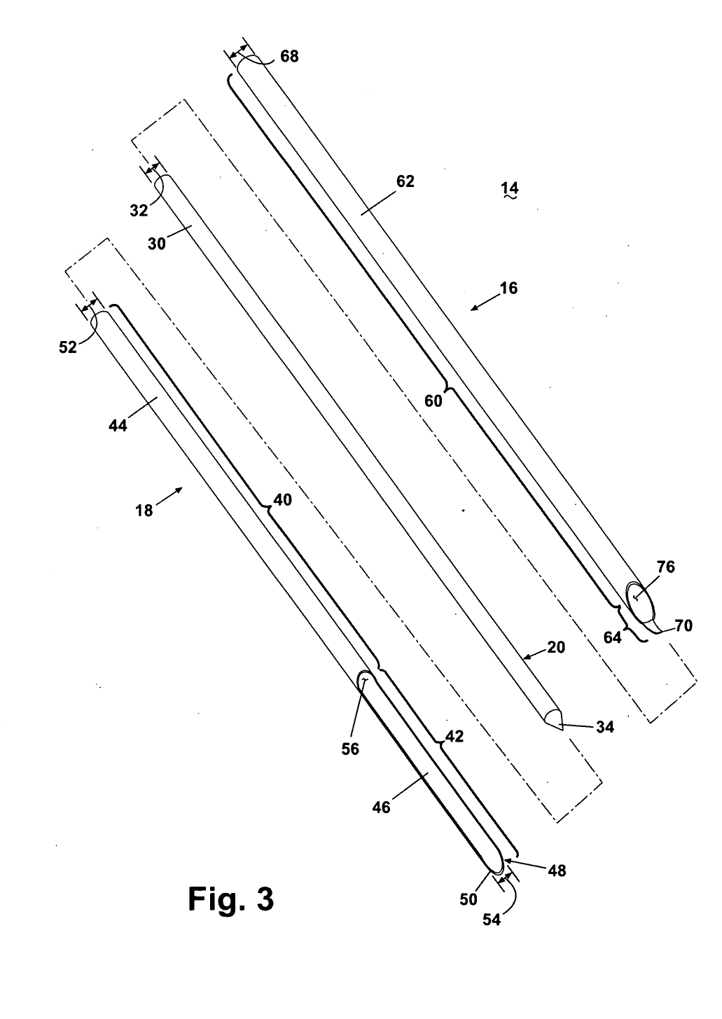

Referring now to FIGS. 2-6, the cannula assembly 14 comprises a coring cannula 16, a spoon cannula 18, and a stylet 20 in coaxially telescoping relationship, as illustrated in FIGS. 2 and 3. As used herein with respect to the coring cannula 16, the spoon cannula 18, and the stylet 20, the terms “distal” and “forward” refer to or in a direction toward that end of the cannulae 16, 18 and/or the stylet 20 that is directed toward the lesion 24 and away from the actuator assembly 12. “Proximal” or “rearward” thus refers to or in a direction toward that end of the cannulae 16, 18 and/or the stylet 20 that is directed away from the lesion 24 and toward the actuator assembly 12. Preferably, the coring cannula 16, the spoon cannula 18, and the stylet 20 are fabricated of a well-known surgically suitable material, such as stainless steel.

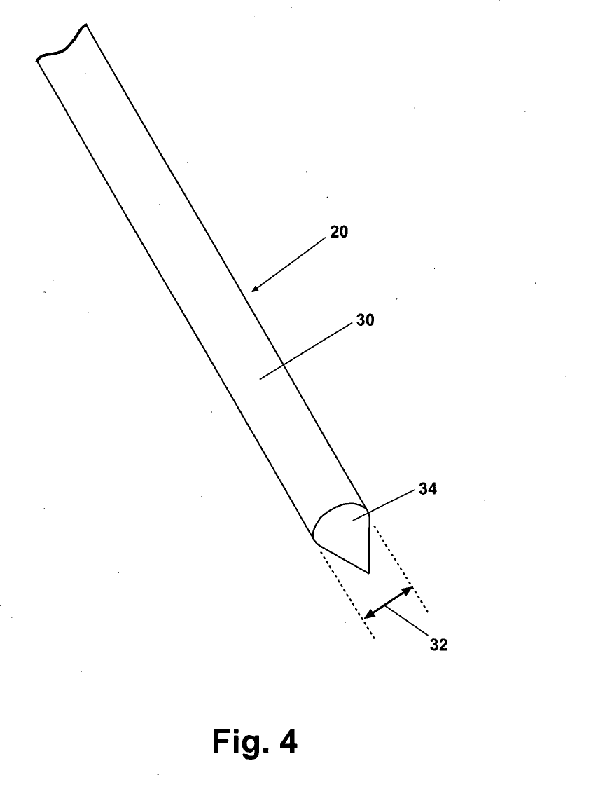

Referring specifically to FIG. 4, the stylet 20 is an elongated, solid cylindrical member comprising a well-known stylet body 30 terminating in a pointed penetration tip 34. The stylet body 30 has a constant stylet diameter 32 which is sized for slidable and coaxial insertion through the spoon cannula 18.

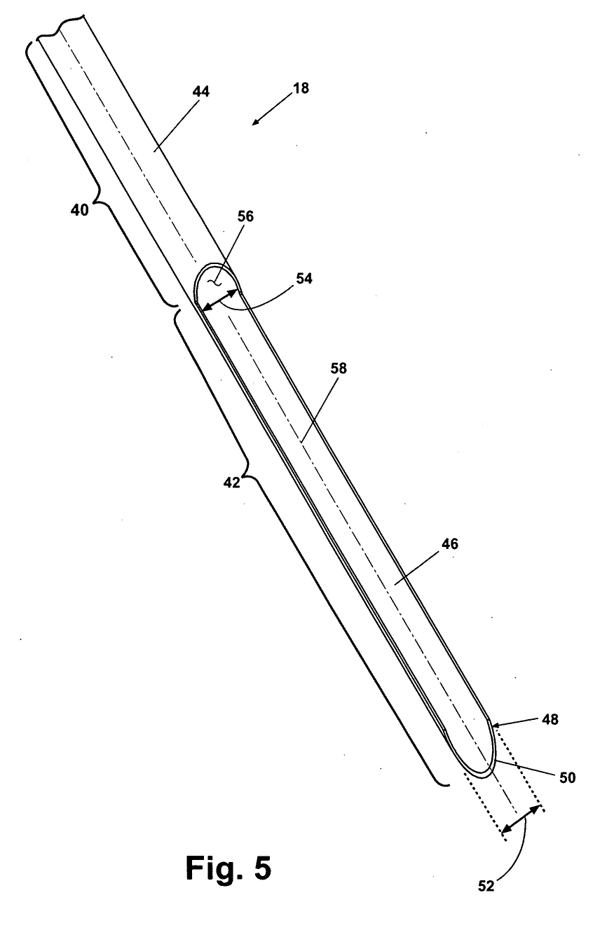

Referring specifically to FIG. 5, the spoon cannula 18 is an elongated, tubular member having an enclosed section 40 smoothly transitioning distally to a spoon section 42. The enclosed section 40 comprises an annular wall 44 having an outer diameter 52 defining a lumen 56 having an inner diameter 54. The spoon section 42 comprises an arcuate wall 46 contiguous with a portion of the annular wall 44. The arcuate wall 46 is preferably semicircular, defining a central angle of 180°. Alternatively, the arcuate wall 46 can comprise an arc length defining a central angle ranging between about 120° and somewhat greater than 180°. An arc length greater than 180° will provide enhanced support of the biopsy sample and will minimize the risk of unintended sample deformation during removal of the sample from the spoon section 42. The inner diameter 54 is somewhat greater than the stylet diameter 32 so that the stylet 20 is slidably received within the lumen 56.

The arcuate wall 46 terminates at a distal end in an insertion tip 48. The distal edge of the arcuate wall 46 at the insertion tip 48 is inclined relative to a longitudinal axis 58 of the spoon cannula 18 to define a parabolic beveled edge 50. The beveled edge 50 can be provided with a secondary bevel, which in effect sharpens the beveled edge 50, to enhance the penetration capability of the spoon cannula 18 into the tissue mass 22 and the lesion 24.

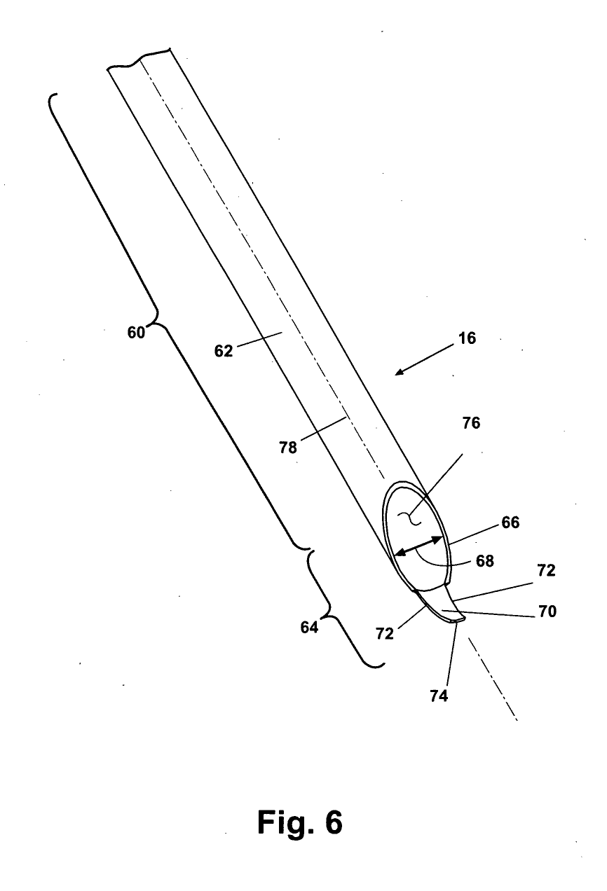

The coring cannula 16, illustrated specifically in FIG. 6, is an elongated, tubular member having an enclosed section 60 comprising an annular wall 62 defining a lumen 76 therethrough having an inner diameter 68. The coring cannula 16 terminates at a distal end in a cutting tip 64. The cutting tip 64 is inclined relative to a longitudinal axis 78 of the coring cannula 16 to define an elliptical beveled edge 66. The beveled edge 66 can be provided with a secondary bevel, which in effect sharpens the beveled edge 66, to enhance the penetration capability of the coring cannula 16 into the tissue mass 22 and the lesion 24.

The inner diameter 68 of the coring cannula 16 is somewhat greater than the outer diameter 52 of the spoon cannula 18 so that the spoon cannula 18 is slidably received within the lumen 76 of the coring cannula 16.

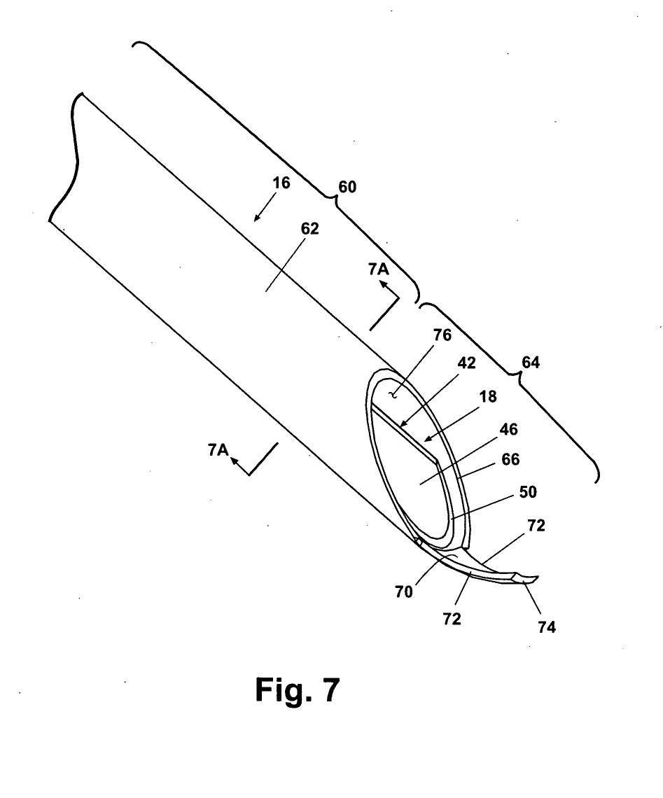

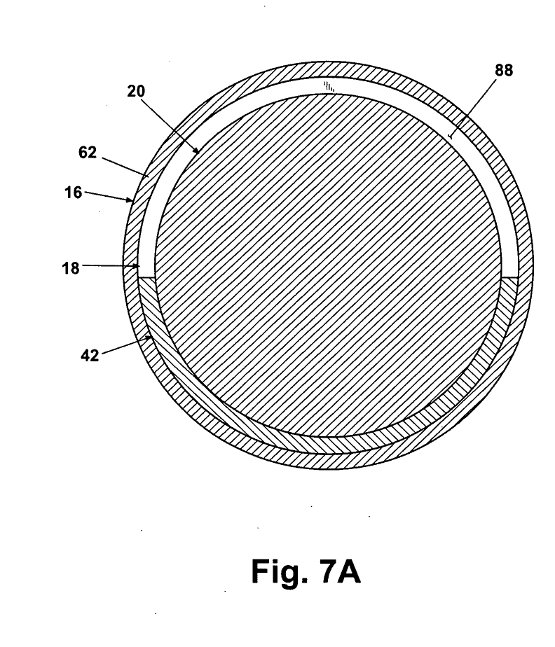

The cutting tip 64 transitions at a distal end to an arcuate excising finger 70 extending generally longitudinally away from the cutting tip 64. The excising finger 70, illustrated in detail in FIG. 7, has a trapezoidal shape comprising a pair of opposed lateral edges 72 terminating in a distal edge 74. Alternatively, the edges 72 can be parallel, providing the excising finger 70 with a rectilinear shape. The edges 72, 74 can be beveled to enhance the penetration and cutting characteristics of the excising finger 70. The excising finger 70 is adapted to have a resilience which enables the excising finger 70 to elastically deflect away from the longitudinal axis 78 and to return to an at-rest arcuate configuration as best seen in FIG. 7.

As illustrated in FIG. 7A, the semi-circular configuration of the spoon section 42 results in a semi-annular gap 88 being defined between the stylet 20 and the annular wall 62 of the coring cannula 16 when the coring cannula 16, the spoon cannula 18, and the stylet 20 are in telescoping relationship. This gap 88 extends longitudinally from the insertion tip 48 to the enclosed section 40.

As illustrated in FIGS. 6, 7, and 8C, the excising finger 70 is curved inwardly toward the longitudinal axis 78 so that the distal edge 74 extends to, and preferably somewhat beyond, the longitudinal axis 78. The extension of the excising finger 70 at least to the longitudinal axis 78 will enable the excising finger 70 to completely excise a biopsy sample from the lesion 24 when the coring cannula 16 is rotated.

Alternatively, the excising finger 70 can extend just short of the longitudinal axis 78 so that a portion of the biopsy sample remains connected to the lesion 24. In such a configuration, care must be taken to ensure that the suction and/or frictional force acting on the biopsy sample in the core biopsy device 10, along with interference from the excising finger 70 on the sample, will exert sufficient force to retain the biopsy sample in the device 10 and separate the remainder of the biopsy sample from the tissue mass 22 as the device 10 is removed.

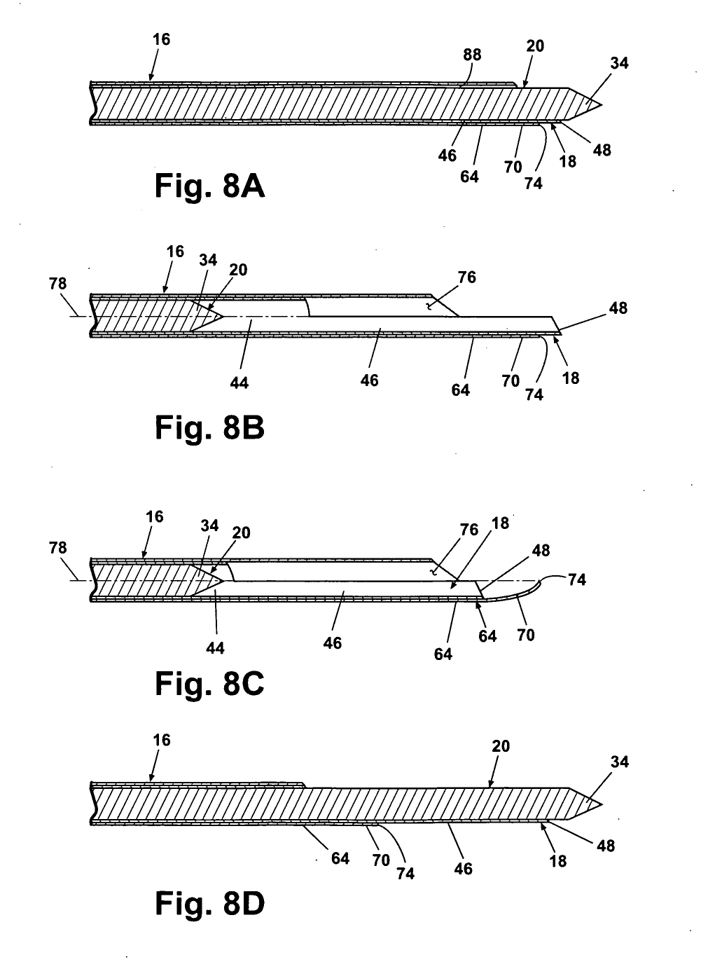

Referring to FIGS. 3 and 8A-C, the cannula assembly 14 is assembled by installing the stylet 20 into the lumen 56 of the spoon cannula 18, and installing the spoon cannula 18 into the lumen 76 of the coring cannula 16, to provide a telescoping assembly wherein the coring cannula 16 is slidably and coaxially disposed around the spoon cannula 18, which is slidably and coaxially disposed around the stylet 20. FIGS. 8A-C illustrate the various relative positions of the elements of the cannula assembly when it is moved from the cocked position (FIG. 8A) to an excising position (FIG. 8C).

As illustrated in FIG. 8A, when the cannula assembly 14 is operably attached to the actuator assembly 12 and placed in a cocked configuration, the penetration tip 34 of the stylet 20 extends somewhat distally of the insertion tip 48 of the spoon cannula 18. The insertion tip 48 also extends somewhat distally of the cutting tip 64 of the coring cannula 16 so that the distal edge 74 of the excising finger 70 is in resilient contact with the arcuate wall 46 of the spoon cannula 18, deflected away from the longitudinal axis 78.

FIG. 8B illustrates the cannula assembly in an intermediate position between the cocked and excising positions. To achieve the intermediate position, after the cannula assembly 14 has been inserted into the tissue mass 22 to the lesion 24, the cannulae 16, 18 can be moved relative to the stylet 20 such that the distal ends of the cannulae 16, 18 extend beyond the penetration tip 34 of the stylet 20 with the insertion tip 48 of the spoon cannula 18 remaining extended distally of the excising finger 70 of the coring cannula 16. This intermediate position can be either a static or dynamic position. It is preferred that this is a dynamic position that is reached as part of the overall movement from the cocked to the excising position.

As illustrated in FIG. 8C, the coring cannula 16 is projected distally of the spoon cannula 18 so that the excising finger 70 extends distally of the insertion tip 48 to deflect arcuately toward the longitudinal axis 78. This configuration is referred to herein as the excising position. While in the excising position, the rotation of the coring cannula 16 and the excising finger 70 relative to the biopsy sample excises the sample from the tissue mass 22.

Referring again to FIG. 8A, as described above with respect to FIG. 7A, the configuration of the spoon cannula 18 comprising the elimination of a portion of the annular wall 44 to form the spoon section 42 results in the semi-annular gap 88 between the stylet 20 and the annular wall 62 of the coring cannula 16 when the coring cannula 16 is extended over the stylet 20 distally of the annular wall 44. This gap is approximately equal to the thickness of the annular wall 44, or, in a preferred embodiment, approximately 0.004 inch. It is contemplated that this gap is insufficient in size for tissue to be received within the gap during the insertion of the cannula assembly into the tissue mass. However, if tissue were received within the gap, it might negatively impact the performance of the biopsy device 10.

FIG. 8D illustrates an alternate position of the coring cannula 16 in the “cocked” configuration that eliminates the gap during the insertion of the cannula assembly into the tissue mass. As shown in FIG. 8D, the enclosed section 60 of the coring cannula 16 is coextensive with the enclosed section 40 of the spoon cannula 18, but does not extend distally beyond it. This configuration will enable the insertion of the cannula assembly 14 into the tissue mass 22 and the simultaneous advancement of the cannulae 16, 18 over the stylet 20 without the presence of a gap or interference from tissue drawn therein. Thus, the penetration tip 34 of the stylet 20 extends somewhat distally of the insertion tip 48 of the spoon cannula 18, but the insertion tip 48 does not extend distally of the cutting tip 64 of the coring cannula 16, which engages the spoon section 42 somewhat proximally of the insertion tip 48. The cannula assembly 14 is inserted into the tissue mass 22 so that the penetration tip 34 and the insertion tip 48 extend to the lesion 24. It will be recognized that the spoon section 42 will necessarily be of a sufficient strength to enable the spoon section 42 to penetrate the tissue mass 22 without deflection, which would be otherwise controlled by the envelopment of the spoon section 42 by the coring cannula 16.

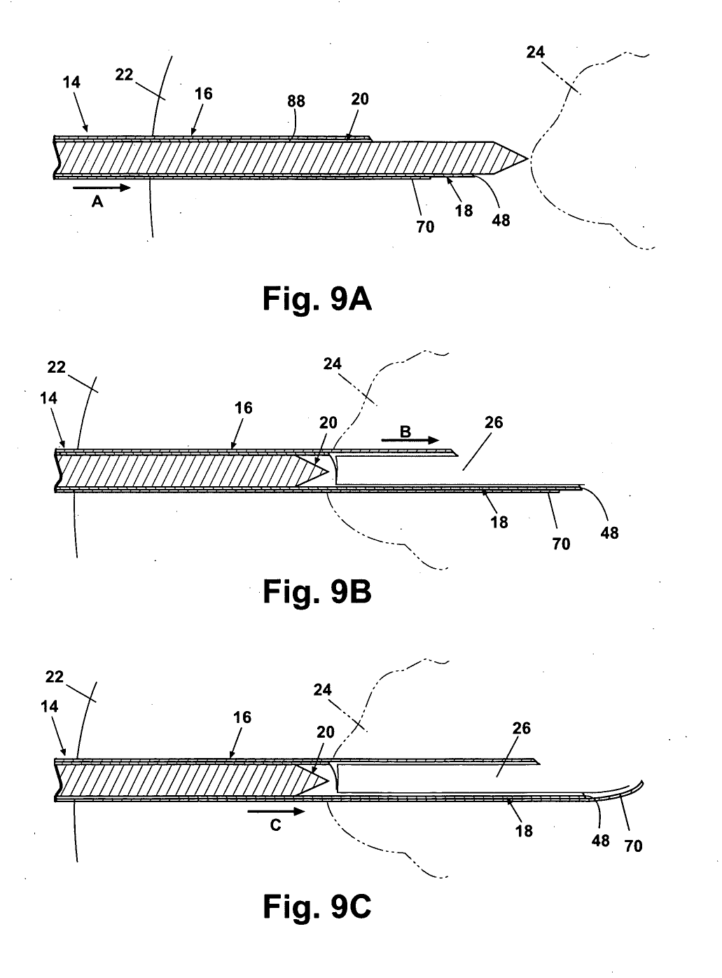

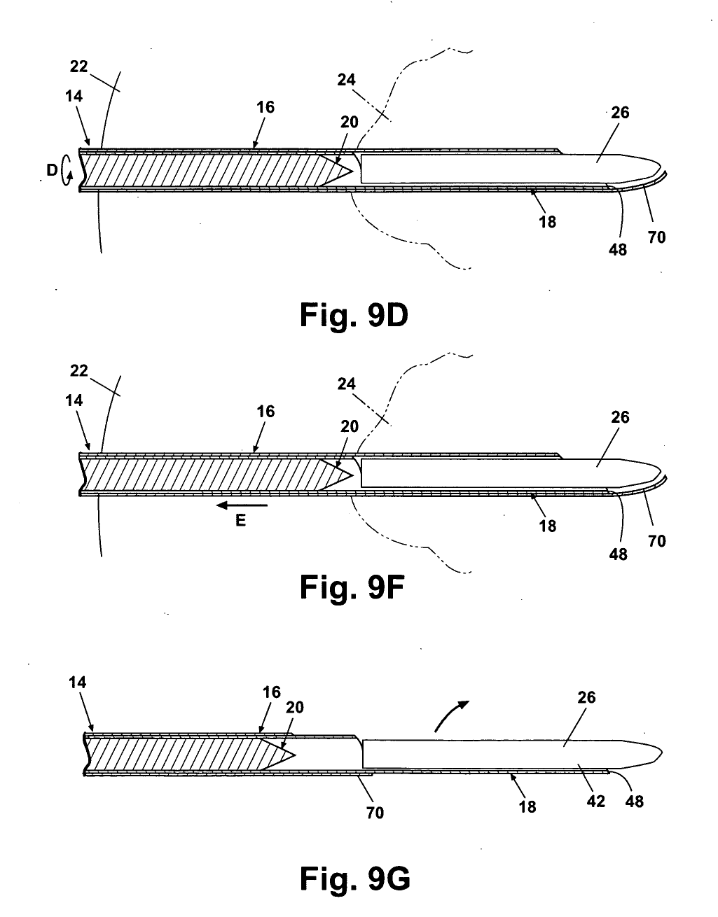

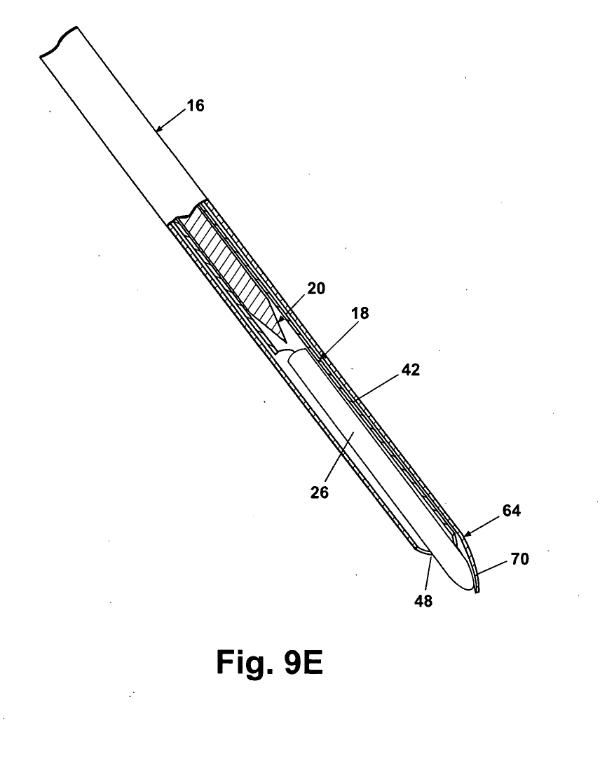

The operation of the core biopsy device 10 is illustrated in FIGS. 9A-F in the context of performing a breast biopsy. However, the core biopsy device 10 is not so limited, and can be utilized to obtain a core biopsy sample from other soft tissues, for example the liver, kidney, or skeletal muscles. As illustrated in FIG. 9A, the biopsy procedure is preferably initiated with the cannula assembly 14 in the cocked configuration. In this configuration, the cannula assembly 14 is inserted into the tissue mass 22 by manual or automated means. Preferably, the user grasps the actuator assembly 12 and inserts the cannula assembly 14 into the tissue mass 22 in the direction of arrow “A” using an imaging system to guide the positioning of the cannula assembly 14. Generally, the cannula assembly 14 is positioned within the tissue mass 22 such that, when the sample is taken, at least part of the lesion 24 is included in the sample.

Any suitable imaging system can be used, for example radiography, ultrasound, or MRI. As is well known in the art, the tip of the stylet, cannula, or spoon cannula can be made from material, shaped or provided with markings that enhance the visibility of the elements with a particular imaging system.

The penetration tip 34 of the stylet 20 extends somewhat distally of the insertion tip 48 of the spoon cannula 18 and the insertion tip 48 extends somewhat distally of the excising finger 70 to form a generally solid penetrating tip that facilitates the insertion of the cannula assembly 14 into the tissue mass 22. Portions of the cannula assembly 14 are preferably made such that they are easily viewable and positionable using the selected imaging technique.

Referring to FIG. 9B, after the initial positioning of the cannula assembly 14, the cannula assembly is moved from the cocked to the excising condition. As part of this movement, the cannula assembly passes through the intermediate position. To affect this movement, the coring cannula 16 and the spoon cannula 18 are advanced relative to the stylet 20, preferably by axially sliding the coring cannula 16 and spoon cannula 18 in the direction of the arrow B along the stylet 20 and into the lesion 24 a distance predetermined by the desired length of the biopsy sample through activation of the actuator assembly 12. In the intermediate position, the excising finger 70 still remains behind the distal end of the spoon and the sample 26 is being cored from the tissue mass 24.

As illustrated in FIG. 9C, after the partial creation of the sample 26 with its end connected to the tissue mass 22, the coring cannula 16 is advanced to the excising position to complete the coring of the sample 26. In the excising position, the excising finger 70 extends beyond the distal end of the spoon and into the end of the sample. This movement is accomplished by moving the coring cannula 16 in the direction of the arrow C. As the excising finger 70 extends beyond the insertion tip 48, the inherent resilience and memory of the excising finger 70 causes it to resiliently return to its at-rest arcuate position, biased in the lesion 24 toward the longitudinal axis 78.

The advancement of the coring cannula 16 to the excising position can be done as part of or separate from the initial advancement of the coring cannula 16 and the spoon cannula 18. Preferably, the advancement of the coring cannula 16 is accomplished in the same step as the advancement of the spoon cannula 18 to form the sample core. To accomplish such a motion, the advancement of the spoon cannula 18 can be stopped prior to the advancement of the coring cannula 16. In other words, the spoon cannula 18 would have a shorter throw distance than the coring cannula 16.

Referring to FIGS. 9D-E, with the coring cannula 16 in the excising position, the sample 26 is severed from the surrounding tissue mass by rotating the coring cannula 16 relative to the spoon cannula 18. As illustrated in FIG. 9D, the coring cannula is rotated in the direction of arrow D. However, either direction of rotation can be used.

The rotation of the excising finger 70 causes the excising finger to sever the end of the sample 26 from the tissue mass to form a somewhat rounded conical surface at the distal end of the biopsy sample 26. The excising finger 70 is preferably rotated approximately 1½ turns to ensure that the biopsy sample 26 has been completely excised from the lesion 24. However, the rotation need only be sufficient to ensure the separation of the sample 26 from the tissue mass 22, which can include a partial revolution or multiple revolutions.

After excising the biopsy sample 26 from the lesion 24, the cylindrical biopsy sample 26 will be supported by the spoon section 42 within the lumen 76, held in place partly by the friction of the cylindrical surface of the sample 26 against the arcuate wall 46 and the annular wall 62, and the radially-inward position of the excising finger 70. With this configuration, the sample 26 is retained within the coring cannula 16 upon removal from the tissue mass 22 by withdrawing the cannula assembly in the direction of arrow E.

Referring to FIG. 9F, after the cannula assembly is withdrawn from the tissue mass, the biopsy sample 26 is removed from the core biopsy device 10 by relatively moving the coring cannula 16 and the spoon cannula 18 such that the spoon section 42 extends beyond the cutting tip 64 of the coring cannula 16. In this position, the sample 26 extends beyond the coring cannula 16 while still being supported by the spoon section 42. The excising finger 70 is also located behind the distal end of the spoon. The practitioner can then lift the sample 26 away from the spoon section 42. This is a great advantage over prior art full core biopsy devices that use the advancement of the stylet to expel the sample. The forced expulsion of the sample can damage the sample and in some cases can render the sample unusable. The biopsy device 10 of the invention can be configured to advance the stylet 20 to expel the sample 26 as do the prior art devices, but for the reasons just stated, it is highly undesirable.

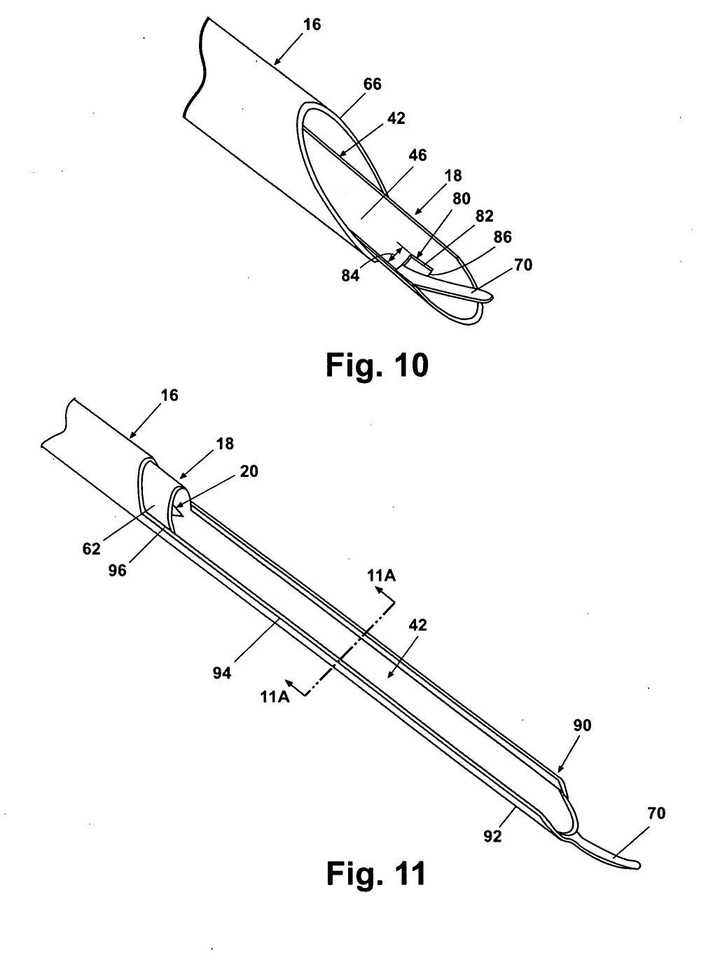

FIG. 10 illustrates a second embodiment of the spoon cannula 18 in which the spoon section 42 is provided with an excising finger window 80 adjacent the insertion tip 48. The excising finger window 80 is a generally rectilinear opening in the arcuate wall 46 comprising a pair of parallel, spaced-apart longitudinal edges 82 defining a window width 84 adapted for slidable insertion of the excising finger 70. The excising finger window 80 has a distal edge 86 adjacent the insertion tip 48. The spoon cannula 18 is received as previously described in the lumen 76 for slidable movement of the spoon cannula 18 relative to the coring cannula 16.

The excising finger 70 is adapted for insertion through the excising finger window 80 so that the excising finger 70 bears against the distal edge 86. As the spoon cannula 18 is drawn into the coring cannula 16, the distal edge 86 bearing against the excising finger 70 will urge the excising finger 70 radially inwardly toward the longitudinal axis 76. Conversely, as the spoon cannula 18 is moved distally along the lumen 76, the finger 70 will resiliently return to its at-rest position. Because the distal edge 86 deflects the excising finger 70, the curvature of the excising finger 70 in its at-rest position can be much shallower, even approaching a straight line, thereby facilitating penetration of the excising finger 70 into the tissue 22 and the lesion 24. With a shallower curvature, the excising finger 70 will be less likely to deflect laterally during penetration. The deflection of the excising finger 70 by the movement of the spoon cannula 18 will enable the excising finger 70 to completely excise the biopsy sample with rotation of the coring cannula 16. It will be evident that rotation of both the coring cannula 16 and the spoon cannula 18 will be required for this embodiment, whereas in the first embodiment only the coring cannula 16 must be rotated.

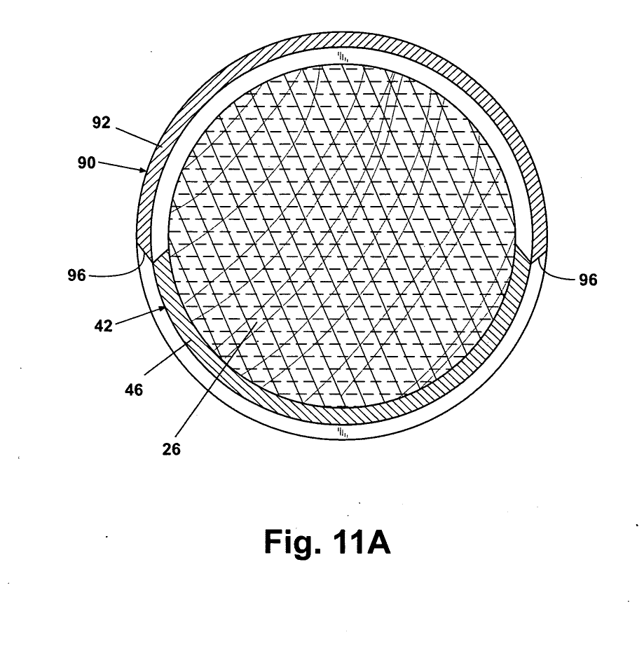

FIG. 11 illustrates a second embodiment of the coring cannula 16 in which the coring cannula 16 is provided with a spoon section 90 similar to the spoon section 42 of the spoon cannula 18. The spoon section 90 comprises an arcuate wall 92 smoothly transitioning distally from the annular wall 62 and cooperatively disposed relative to the arcuate wall 46. The arcuate wall 92 terminates in a pair of parallel, longitudinal, juxtaposed edges 94 extending from the annular wall 62 to the cutting tip 64. The edges 94 are provided with a sharpened bevel 96 adapted for excision of a biopsy sample. After the coring cannula 16 and the spoon cannula 18 have been projected into the lesion 24, as described above, the coring cannula 16 is rotated to excise a biopsy sample from the lesion 24 by the cutting action of the bevel 96 and the excising finger 70. The coring cannula 16 will be rotated so that the annular wall 92 is diametrically disposed relative to the annular wall 44 to form a generally enclosed tubular sample retaining cavity (FIG. 11A).

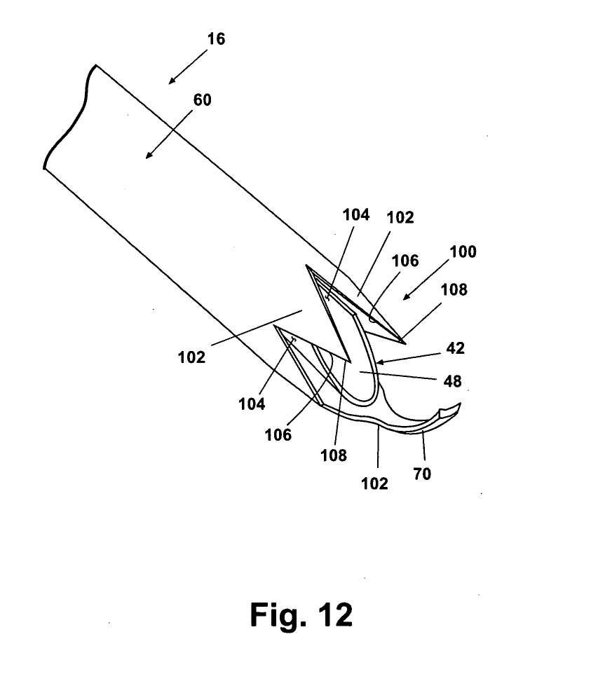

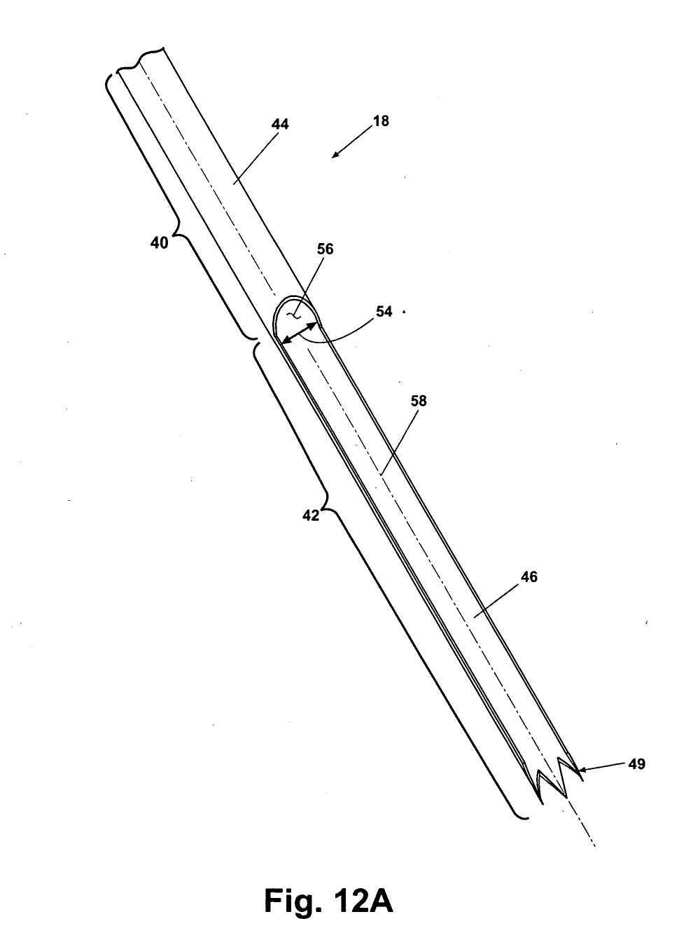

FIG. 12 illustrates an embodiment of the coring cannula 16 in which the cutting tip 64 is provided with a scalloped edge 200 to facilitate penetration of the cutting tip into the tissue 22 and the lesion 24. The scalloped edge 200 defines a plurality of teeth 202 separated by a plurality of valleys 204. The teeth 202 and the valleys 204 can be provided with a sharpened beveled tooth edge 206 for incising into the lesion 24. Each tooth 202 terminates distally in a crown 208. One of the teeth 202 is extended distally of the crown 208 to smoothly transition into the excising finger 70. The spoon cannula 18 can also be provided with a scalloped edge along the insertion tip 48 (FIG. 12A). The insertion of the cannula assembly 14 and the excision of a biopsy sample 26 are generally as previously described herein.

The core biopsy device 10 can be operated in a “corkscrew” manner by rotating the cutting cannula while it is inserted or retracted to excise a biopsy sample that is incised helically and can be unrolled into a generally flat sample. However, due to the increased complexity in obtaining a satisfactory helical sample, this procedure is not preferred.

The actuator assembly can be of any suitable construction as long as it can longitudinally extend the coring and spoon cannulae 16, 18, simultaneously or sequentially in a controlled manner, to place the coring cannula 16 in the excising position, and then rotate the coring cannula 16 a sufficient amount to ensure the separation of the sample 26 from the tissue mass 22 upon withdrawal of the cannula assembly 14.

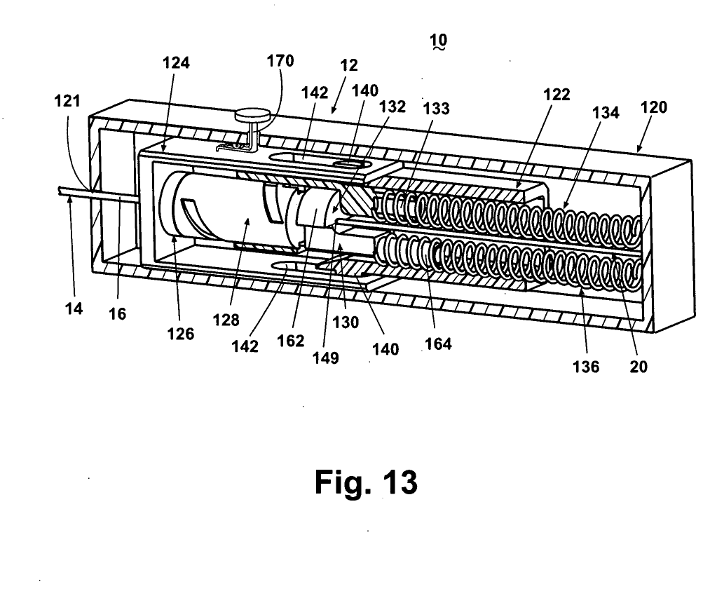

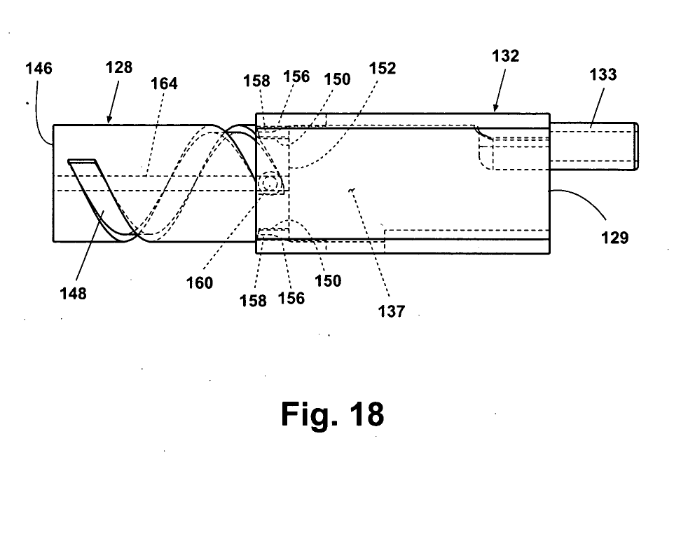

FIGS. 13-23 illustrate a first embodiment of an actuator assembly 12 for both translating the cannula assembly 14 to the excising position and then rotating the coring cannula 16. As used herein, the term “distal” or “forward” refers to or in a direction toward that end of the actuator assembly 12 and its component parts that is directed toward the cannula assembly 14. “Proximal” or “rearward” thus refers to or in a direction toward that end of the actuator assembly 12 and its component parts that is directed away from the cannula assembly 14.

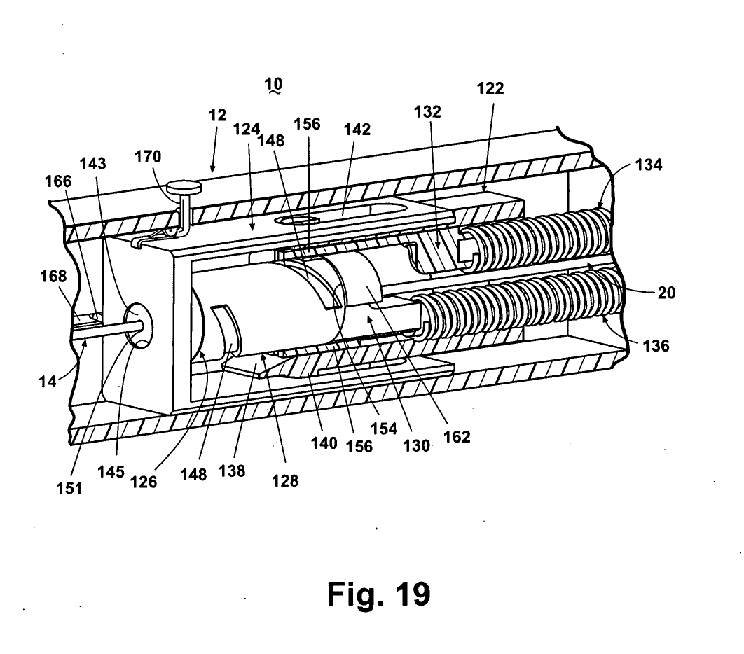

FIG. 13 illustrates the actuator assembly 12 comprising an actuator enclosure 120 having a portion removed along a longitudinal medial plane for convenience in illustrating and describing the structure and operation of the internal components. The actuator enclosure 120 is illustrated as a generally box-like structure provided with an aperture 121 through which the cannula assembly 14 can slidably extend. The actuator assembly 12 also functions as a handle for the biopsy device 10. Thus, the actuator enclosure 120 can, and preferably will, have a shape which can accommodate the internal components while providing suitable ergonomics for a user to readily and comfortably grip and operate the device. As illustrated in FIGS. 13 and 19-22, the enclosure 120 is provided with a trigger mechanism 170 for initiating the firing and rotation of the cannula assembly 14 into the lesion, and a cocking lever 166 extending through a slot 168 in a wall of the enclosure 120 (FIGS. 19-21) for setting the actuator assembly 12 into the “ready to fire” position, which corresponds to the cocked position for the cannula assembly. The trigger mechanism and cocking lever are illustrated for exemplary purposes only, and other suitable mechanisms for setting and initiating the operation of the actuator assembly can be utilized.

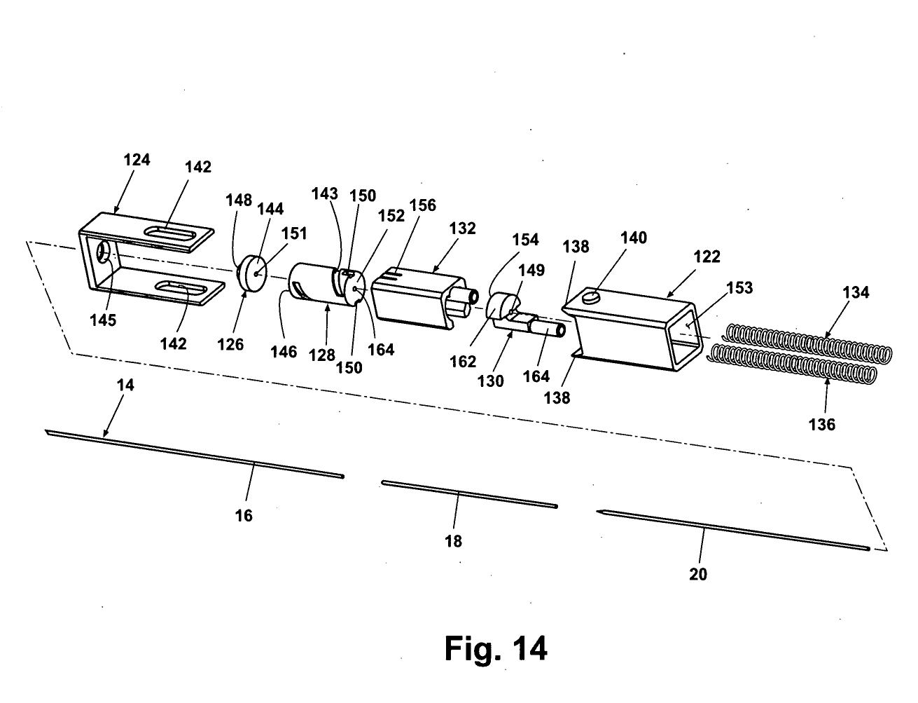

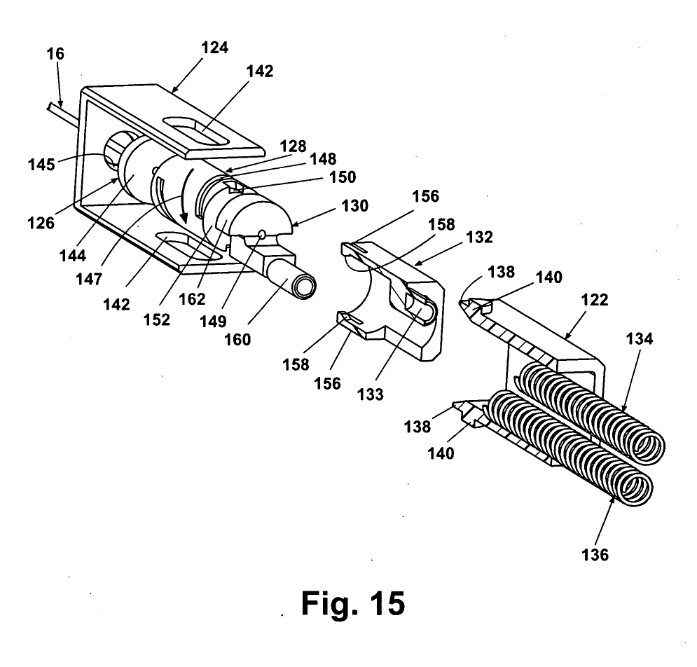

Referring also to FIGS. 14 and 15, the actuator enclosure 120 encloses a retraction body 122 which is fixedly attached thereto. The actuator enclosure 120 also encloses a translating body 124 adapted for linear movement relative to the actuator enclosure 120 and the retraction body 122, a clutch 126, a rotating cylinder 128, a cannula block 130, a driving sleeve 132, a rotation spring 134, and a drive spring 136.

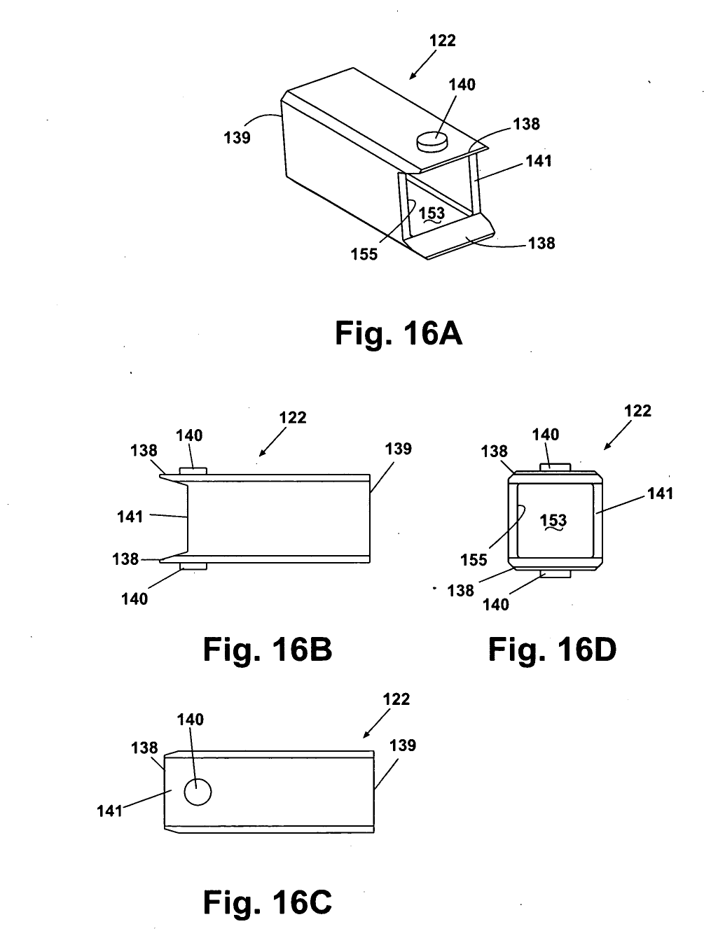

Referring to FIGS. 16A-D, the retraction body 122 is a generally rectilinear, box-like body having a generally square cross-section, an open proximal end 139, and an open distal end 141. The distal end 141 terminates in a pair of opposed, wedge-shaped flanges 138, and a pair of opposed stop bosses 140 extending laterally outwardly therefrom. A rectilinear duct 155 extends longitudinally through the retraction body 122 to define a driving sleeve opening 153.

Referring to FIGS. 14 and 15, the translating body 124 is a rectilinear, generally U-shaped body having a pair of opposed slots 142 at a proximal end thereof. The slots 142 are sized for slidable register with the stop bosses 140. The clutch 126 is a circular, plate-like body having an axial cylindrical stub shaft 143 adapted for fixed reception into a mating circular opening 145 in the translating body 124. The clutch 26 has a clutch face 144 at a proximal end thereof, and is provided with a circular clutch aperture 151 extending axially therethrough and adapted for rotation of the cannula assembly 14 therein.

Referring to FIGS. 14, 15, and 18, the rotating cylinder 128 is a generally solid cylindrical body comprising a helical slot 148 extending therearound and having a cylinder aperture 164 extending axially therethrough adapted for fixed communication with the coring cannula 16 at a distal end thereof and for rotation of the spoon cannula 18 proximal of the coring cannula 16. The rotating cylinder 128 is provided with a first cylinder face 146 at a distal end thereof adapted for operable register with the clutch face 144, and a second cylinder face 152 at a proximal end thereof. The coring cannula 16 is fixedly received in the cylinder aperture 164. A pair of diametrically opposed detents 150 extend radially inwardly at a proximal end thereof.

As illustrated in FIGS. 14, 15, and 19, the cannula block 130 is an irregularly shaped body comprising a semi-circular shoe 162 having a cannula block face 154 at a distal end thereof, and a coaxial stylet aperture 149 adapted for coaxial fixed communication with the proximal end of the spoon cannula 18 therein. The spoon cannula is fixedly mounted within the stylet aperture. The cannula block 130 is also adapted with a columnar spring support 164 which is inserted into the drive spring 136 to retain the drive spring 136 in a selected operable configuration.

The stylet 20 extends through the rotating cylinder 128 and the cannula block 130 for fixed engagement with the actuator enclosure 120, such as by seating a proximal end of the stylet 20 into a proximal end wall of the actuator enclosure 120, so that the stylet is fixed against movement relative to the cannulae 16, 18.

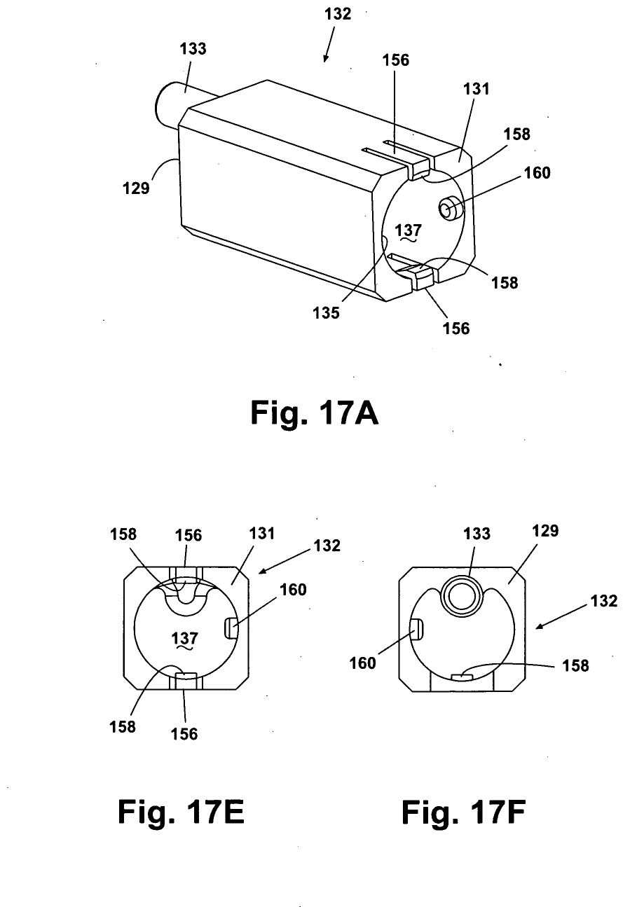

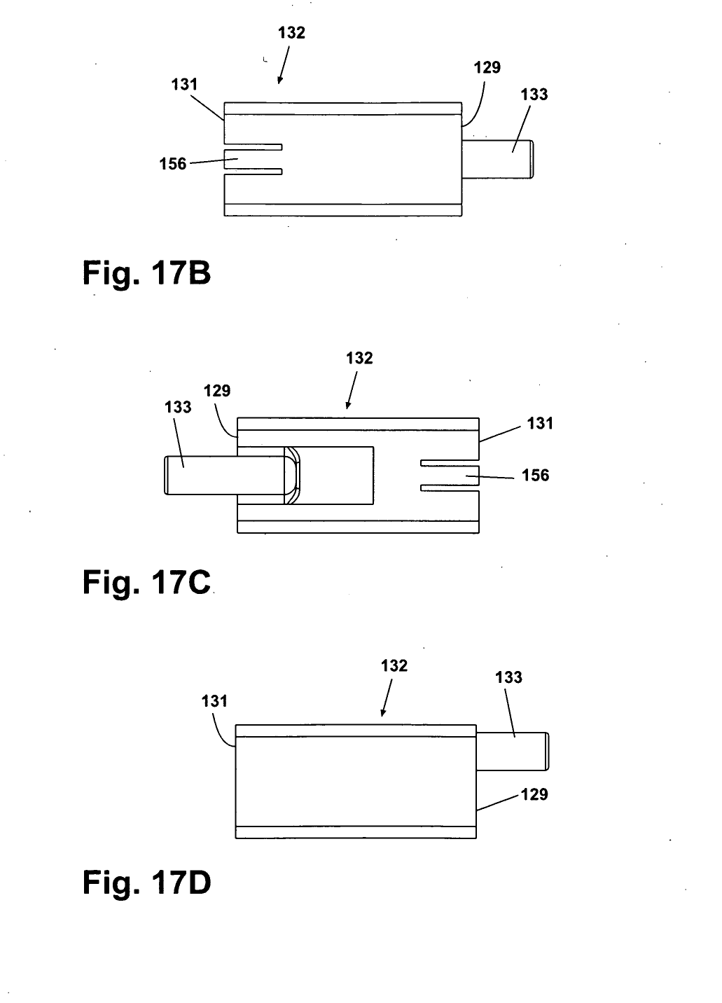

As illustrated in FIGS. 14 and 17A-F, the driving sleeve 132 is a generally rectilinear-shaped body having a generally square cross-section, and a circular coaxial bore 135 defining a cylinder passageway 137 therethrough. The driving sleeve 132 has an open proximal end 129, and an open distal end 131, and the cylinder passageway 137 is adapted for partial slidable envelopment of the cannula block 130 and the rotating cylinder 128. The distal end 131 terminates in a pair of opposed cantilevered flexure flanges 156. The flexure flanges 156 are biased to an outward position and comprise a pair of radially inwardly-directed, diametrically-opposed bosses 158 adapted for cooperating register with the detents 150. The driving sleeve 132 is also provided with a radially inwardly-directed rotation pin 160 adapted for cooperating register with the helical slot 148 and preferably spaced 90 degrees from the bosses 158. The driving sleeve 132 is also adapted with a columnar spring support 133 which is inserted into the rotation spring 134 to retain the rotation spring 134 in a selected operable configuration. Both the rotation spring 134 and the drive spring 136 are fixedly attached to the actuator enclosure 120, such as by columnar spring supports (not shown), sockets (not shown), or other suitable means, such as welding or an adhesive.

The cannula block 130 and the driving sleeve 132 are adapted for independent linear translation, but not rotation. The rotating cylinder 128 is also capable of linear translation independent of the cannula block 130, and is adapted for counterclockwise rotation, as illustrated by the rotation vector 147 in FIG. 15. The clutch face 144 and the cylinder face 146 are adapted for cooperating register to enable counterclockwise rotation of the rotating cylinder 128 but prevent clockwise rotation of the rotating cylinder 128, such as by a ratchet-type engagement that allows relative rotation in only one direction.

Referring again to FIGS. 13-15, the clutch 126 is fixedly attached to the translating body 124 by inserting the stub shaft 143 into the opening 145, with the cannula assembly 14 extending coaxially through the clutch aperture 151 for rotation of the cannula assembly 14 relative to the clutch 126. With the coring cannula 16 fixedly received in the cylinder aperture 164 in the rotating cylinder 128, and the spoon cannula 18 extending therethrough and fixedly received into the cannula block 130, the rotating cylinder 128 and coring cannula 16 can rotate about the spoon cannula 18. When the cylinder face 146 is in contact with the clutch face 144, the rotating cylinder 128 can rotate in a counterclockwise direction, but not a clockwise direction.

The driving sleeve 132 slidably envelops the rotating cylinder 128 and the cannula block 130 within the cylinder passageway 137 so that the bosses 158 engage the detents 150 and the rotation pin 160 engages the helical slot 148. The driving sleeve 132 with the rotating cylinder 128 and the cannula block 130 are slidably received in the driving sleeve opening 153 of the retraction body 122. The translating body 124 is slidably attached to the retraction body 122 by insertion of the stop bosses 140 in the slots 142.

The operation of the actuator assembly 12 will now be described. Referring again to FIG. 13 and to FIG. 19, as assembled, the translating body 124 can move linearly relative to the retraction body 122 within limits defined by the engagement of the stop bosses 140 with the slots 142. The rotating cylinder 128 and the coring cannula 16 can translate linearly with and independently of the spoon cannula 18, and can rotate independently of the spoon cannula 18. The rotating cylinder 128 can also translate linearly independently of the cannula block 130. When the driving sleeve 132 is received fully into the retraction body 122, the flexure flanges 156 will be urged inwardly so that the bosses 158 engage the detents 150 to prevent the rotation of the rotating cylinder 128. When the driving sleeve 132 translates distally away from the retraction body 122, the inclined faces of the flanges 138 enable the flexure flanges 156 to flex outwardly due to their resilient outward bias so that the bosses 158 disengage from the detents 150 to enable rotation of the rotating cylinder 128.

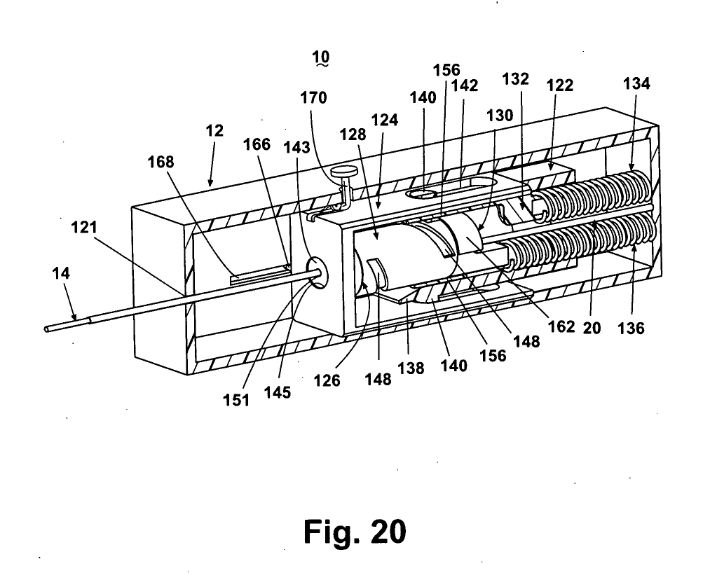

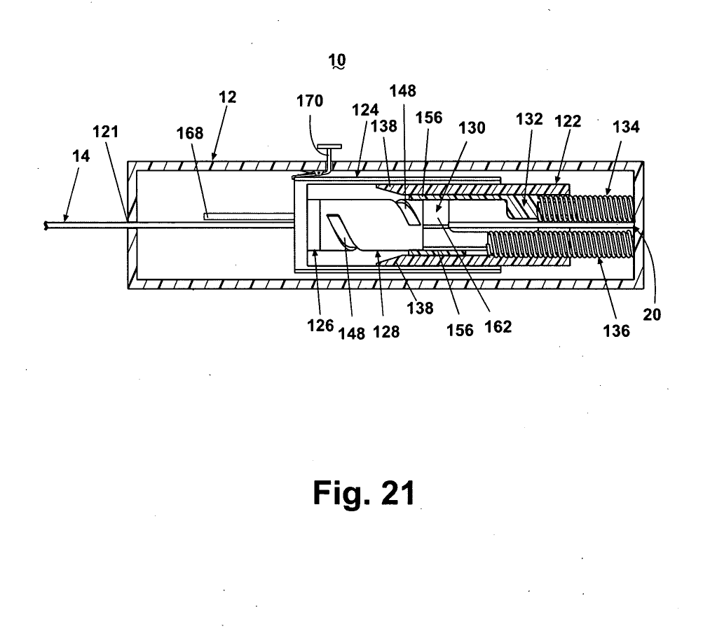

Referring to FIGS. 20 and 21, when the actuator assembly 12 is in a ready position that corresponds to the cocked condition of the cannula assembly, the driving sleeve 132 is fully received in the retraction body 122, which urges the flexure flanges 156 inwardly so that the bosses 158 engage the detents 150, thereby preventing rotation of the rotating cylinder 128. The pin 160 engages the helical slot 148. The cannula block 130 is also received within the retraction body 122 and the springs 134, 136 are compressed. The translating body 124 is held against the spring force of the springs 134, 136 by the trigger 170. The stop bosses 140 engage the distal end of the slots 142 and the rotating cylinder 128 is in contact with both the clutch 126 and the cannula block 130. In this cocked position, as also illustrated in FIG. 8A, the cannulae 16, 18 are retracted proximally so that the penetration tip 34 of the stylet 20 extends somewhat distally of the spoon section 42 of the spoon cannula 18, and the excising finger 70 is in resilient contact with the arcuate wall 46 of the spoon cannula 18 somewhat distally of the insertion tip 48. The enclosed section 60 of the coring cannula 16 extends distally of the enclosed section 48 of the spoon cannula 18 to define the semi-annular gap 88 illustrated in FIG. 7A.

As described previously herein and illustrated in FIG. 9A, with the actuator assembly 12 in the cocked condition, the cannula assembly 14 is inserted into the tissue mass 22 so that the penetration tip 34 is adjacent to the lesion 24. The actuator assembly 12 is then fired by operation of the trigger mechanism 170 for excision of a biopsy sample.

To release the actuator from the ready position to move the cannula assembly from the cocked to the excising position, the trigger 170 is depressed, permitting the translating body to move in response to the spring force. The springs 134, 136 propel the cannula block 130 and the driving sleeve 132 forward, resulting in the corresponding movement of the rotating cylinder 128 forward, which propels the translating body 124 forward. The forward movement of the rotating cylinder 128 and the cannula block 130 advance the coring cannula 16 and the spoon cannula 18, respectively, into the lesion 24 (FIGS. 9B, 22, and 23).

At a point in this movement, the drive spring 136 reaches the limit of its extension, thereby preventing further advancement of the spoon cannula 18. This corresponds to the intermediate position as illustrated in FIG. 9B. During this movement, the rotating cylinder 128 is prevented from rotating by the bosses 158 engaging the detents 150. It is within the scope of the invention to provide a positive stop for the cannula block 130 to stop the advancement of the spoon cannula, instead of relying on the expansion limit of the spring.

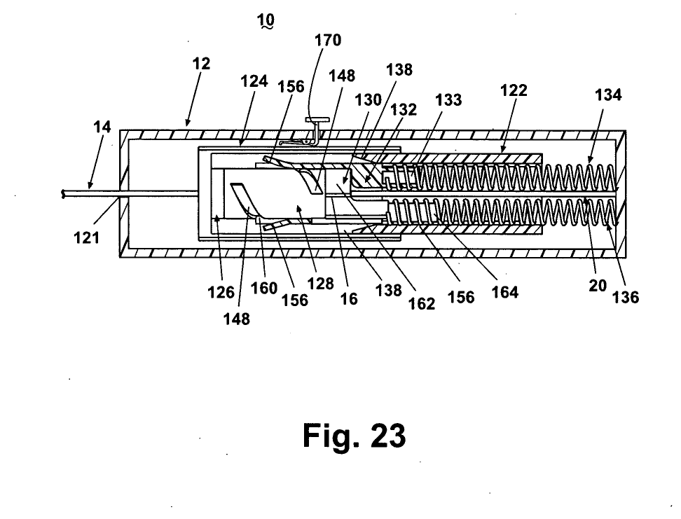

The rotation spring 134 continues to propel the driving sleeve 132 forward to move the cannula assembly into the excising position by moving the excising finger 70 until it clears the insertion tip 48 of the spoon cannula 18 (FIG. 9C). During the movement from the intermediate position to the excising position, the flexure flanges 156 clear the flanges 138, and the bosses 158 disengage from the detents 150 (FIG. 23) to permit the rotation of the rotating cylinder as the excising finger reaches the excising position. It is highly preferred that the rotation of the excising finger not begin until the excising finger has reached its longitudinal extent. This prevents the rotation of the excising finger from cutting a helical path in the sample.

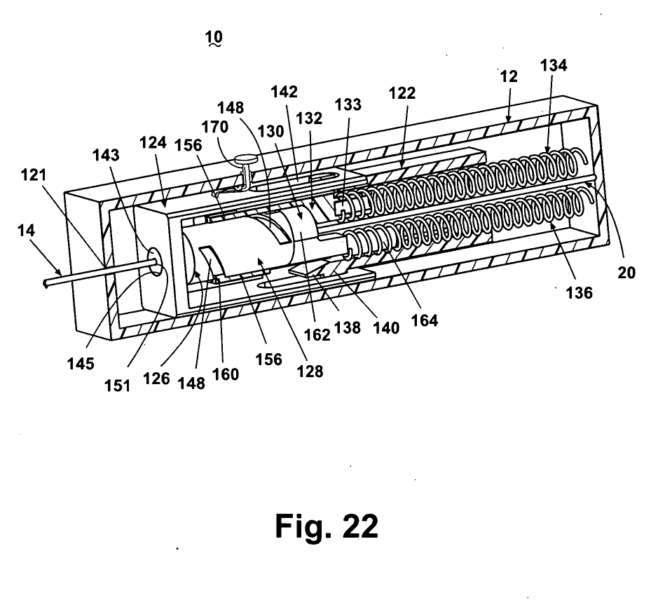

In the excising position, the translating body 124 is prevented from further longitudinal movement by the engagement of the stop bosses 140 with the proximal end of the slots 142 (FIG. 22). Likewise, the rotating cylinder is prevented from further longitudinal movement since it is in contact with the translating body via the clutch 26. However, the driving sleeve 132 can continue to move forward under the influence of the rotation spring 134. Further forward translation of the driving sleeve 132 urges the counterclockwise rotation of the rotating cylinder 128 as the rotation pin 160 travels along the helical slot 148 (FIG. 9D). That is, since the pin 160 is rotationally fixed to the driving sleeve, the forward movement of the driving sleeve causes the helical slot to follow the pin, which necessarily requires the cylinder 128 to rotate in place, without any forward movement. The helical slot 148, the driving sleeve 132, and the rotation pin 160 are preferably adapted so that the rotating cylinder 128 and the coring cannula 16 rotate 1½ turns with a complete stroke of the driving sleeve 132. More than one complete revolution increases the likelihood that the sample is completely severed.

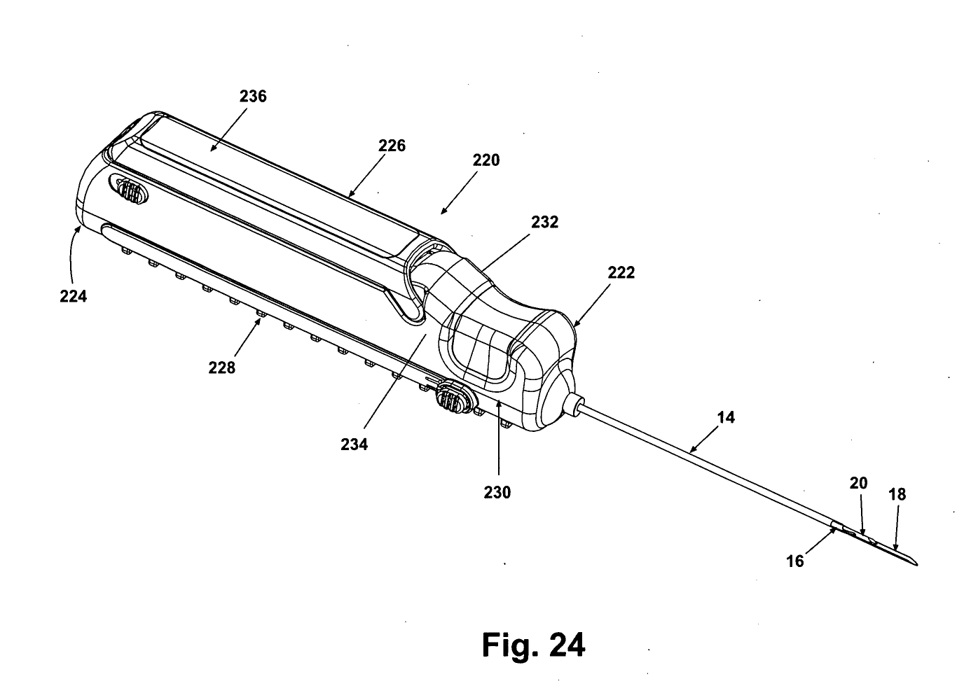

FIGS. 24-49B illustrate a second embodiment of an actuator assembly 220, also referred to as a “biopsy gun,” for both translating the cannula assembly 14 to the excising position and then rotating the coring cannula 16. As used herein, the term “distal” or “forward” refers to or in a direction toward that end of the biopsy gun 220 and its component parts that is directed toward the cannula assembly 14. “Proximal” or “rearward” thus refers to or in a direction toward that end of the biopsy gun 220 and its component parts that is directed away from the cannula assembly 14. Additionally, “dorsal” or “upper” refers to or in a direction toward the top of the biopsy gun 220 when oriented as illustrated in FIG. 24, and “ventral” or “lower” refers to or in a direction toward the bottom of the biopsy gun 220 when oriented as illustrated in FIG. 24.

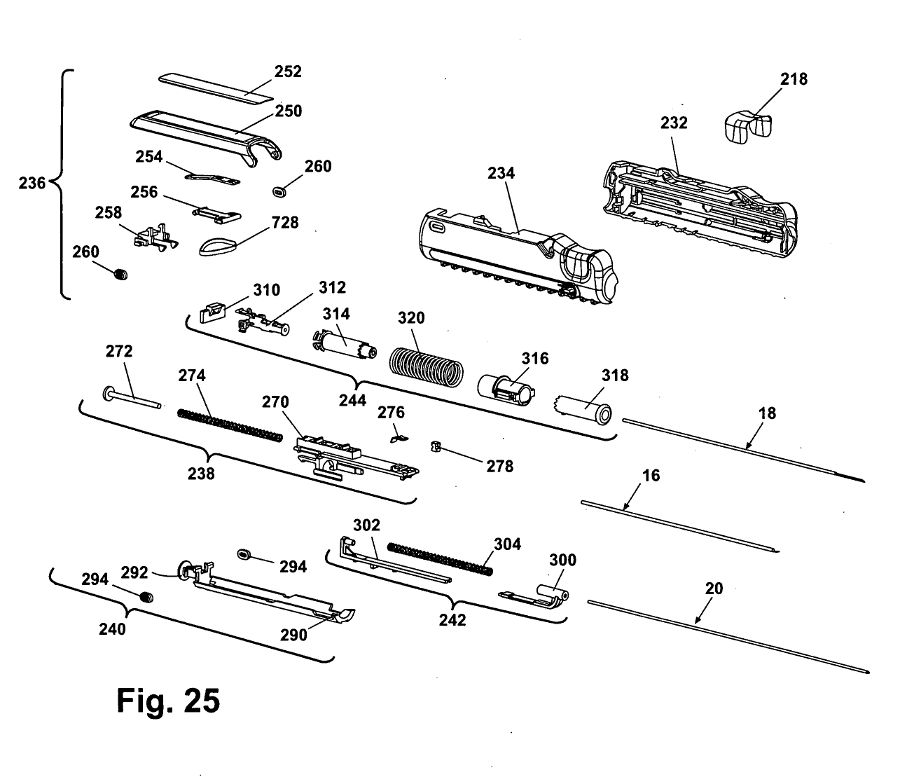

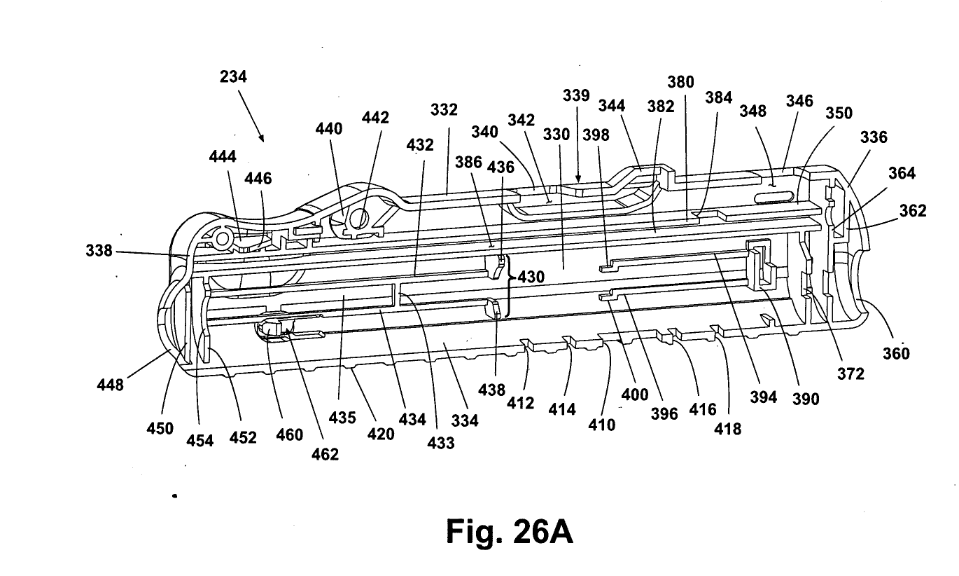

FIG. 24 illustrates the biopsy gun 220 operably connected to the cannula assembly 14. The biopsy gun 220 has a distal end 222, a proximal end 224, a dorsal side 226 supporting a cocking handle assembly 236, and a ventral side 228. Referring also to FIG. 25, the biopsy gun 220 comprises an outer housing 230 comprising a left housing shell 232 and a right housing shell 234 adapted for cooperative registry, and a housing grip 218, to provide an ergonomic, functional handle for facilitating the insertion of the cannula assembly 14 in a lesion 24 and the recovery of a biopsy sample 26.

The biopsy gun 220 comprises the cocking handle assembly 236, a shuttle assembly 238, a trigger assembly 240, a sample size control assembly 242, and a cannula operation assembly 244. The cocking handle assembly 236 comprises a handle 250, a grip pad 252, a spring 254, a swing arm 256, a latch 258, and a pair of latch buttons 260. The shuttle assembly 238 comprises a shuttle 270, a spring retainer 272, a spring 274, a cam spring 276, and a cam block 278. The trigger assembly 240 comprises a firing cage 290 terminating proximally in a firing plunger 292 and a pair of firing buttons 294. The sample size control assembly 242 comprises a nosepiece 300, an adjustment member 302, and a spring 304. The cannula operation assembly 244 comprises a latch plate 310, a spoon cannula carriage 312, a cutting cannula carriage 314, a helical drive member 316, a rotating driven member 318, and a spring 320. These elements are interconnected and supported within the housing 230 in and on various seats, slots, and rails facilitating the precisely controlled movement of the elements during the sample recovery process.

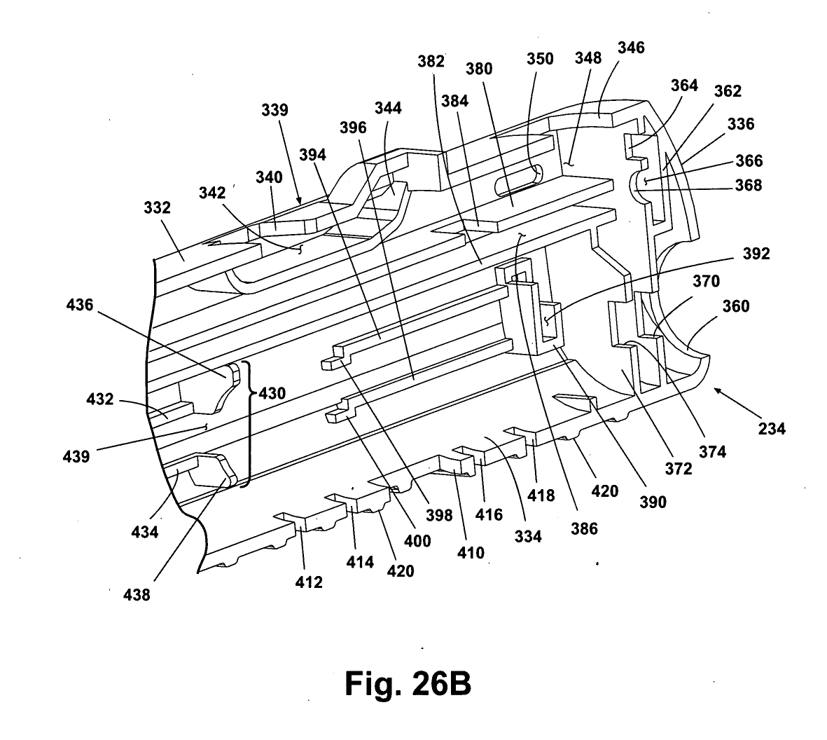

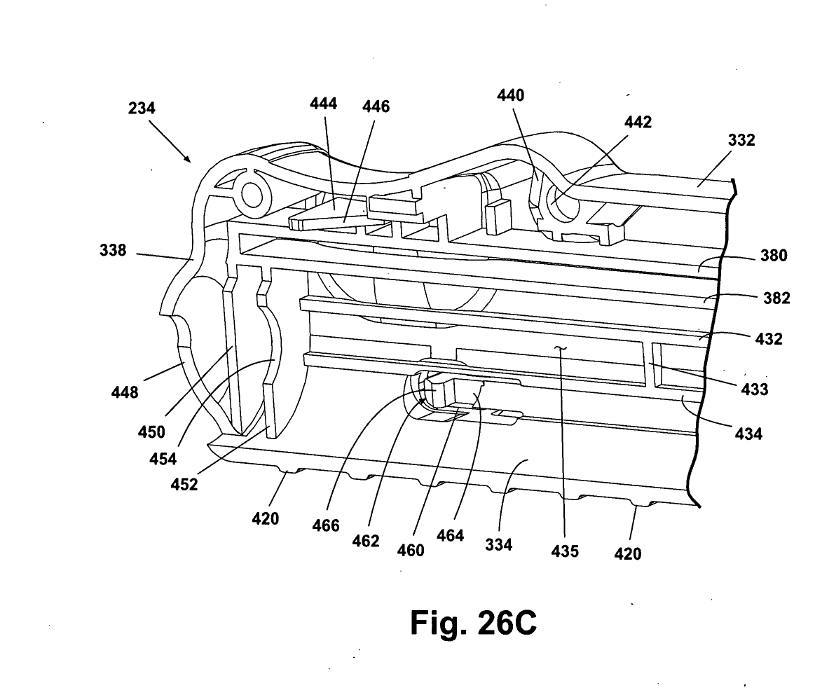

FIGS. 26A-C and 27 illustrate the right housing shell 234. The housing shell 234 is an irregularly-shaped, elongated body comprising an elongated side wall 330 joined to a top wall 332, a bottom wall 334, a proximal wall 336, and a distal wall 338. The walls 330-338 are contoured, and configured with openings, bosses, rails, and the like, for operational support of the elements comprising the biopsy gun 220. Referring specifically to FIG. 26B, the top wall 332 is provided with a swing arm opening 340 therethrough entering into a swing arm cage 339 comprising an elongated, somewhat curved swing arm chamber 342 terminating in a proximal end 344 inclined upwardly away from the swing arm opening 340. Proximal of the swing arm opening 340, the top wall 332 is provided with an upper latch opening 346 therethrough entering into a latch chamber 348. A latch slot 350 communicates with the latch chamber 348 through the side wall 330.

The proximal wall 336 curves somewhat outwardly from the top wall 332 to the bottom wall 334, and is inset with a semicircular trigger opening 360 at a lower portion thereof. Extending inwardly from the side wall 330, and depending from an upper portion of the proximal wall 336, is an abbreviated outer wall 362. Extending inwardly from the side wall 330, and from the top wall 332 to the bottom wall 334, parallel to the outer wall 362, is an intermediate wall 364. An upper portion of the intermediate wall 364 is joined to the outer wall 362 to form a narrow spring retainer chamber 366. The intermediate wall 364 is inset with a semicircular spring retainer opening 368 at an upper portion thereof in communication with the spring retainer chamber 366, and with a rectilinear intermediate trigger opening 370 at a lower portion thereof in axial alignment with the trigger opening 360. Extending inwardly from the side wall 330, and upwardly from the bottom wall 334, parallel to the intermediate wall 364, is an inner wall 372. The inner wall 372 is inset with a rectilinear distal trigger opening 374 in axial alignment with the intermediate trigger opening 370 and the trigger opening 360.

Extending inwardly from the sidewall 330 generally parallel to the top wall 332, and from the intermediate wall 364 toward the distal wall 338, are an upper interior wall 380 and a lower interior wall 382 in parallel, spaced-apart juxtaposition. The upper interior wall 380 is inset with a rectilinear lower latch opening 384 therethrough at a proximal end thereof. The upper interior wall 380 and the lower interior wall 382 define an elongated shuttle slot 386 therebetween.

Extending inwardly from the side wall 330, distally of the inner wall 372 and ventrally of the lower interior wall 382, is a rectilinear latch plate support 390 defining a latch plate channel 392. Extending inwardly from the side wall 330 and distally from the latch plate support 390 are an upper spoon cannula carriage rail 394 and a lower spoon cannula carriage rail 396 in parallel, spaced-apart juxtaposition. The upper spoon cannula carriage rail 394 terminates in an upper stop 398. The lower spoon cannula carriage rail 396 terminates in a lower stop 400. The carriage rails 394, 396 are generally parallel to the upper interior wall 380 and the lower interior wall 382. A cutting cannula carriage cradle 430 comprises an upper cradle piece 436 and a lower cradle piece 438 extending inwardly from an intermediate location on the side wall 330 and defining an arcuate, inward-facing surface. The cradle pieces 436, 438 are separated by a slot 439.

Referring now to FIG. 26C, the upper interior wall 380 and the lower interior wall 382 extend distally to an intermediate wall 450 extending inwardly from the side wall 330 and upwardly from the bottom wall 334. An inner wall 452 extends inwardly from the side wall 330 and upwardly from the bottom wall 334 parallel to the intermediate wall 450. The inner wall 452 is inset with a nosepiece opening 454 therethrough. Referring again to FIG. 26B, an upper rib 432 and a lower rib 434 extend inwardly from the side wall 330 from the cutting cannula carriage cradle 430 to the inner wall 452 in parallel, spaced-apart juxtaposition, ventrally of and generally parallel to the lower interior wall 382 to define a slot 435. A stop rib 433 extends orthogonally from the upper rib 432 to the lower rib 434 intermediate the ends of the ribs 432, 434.

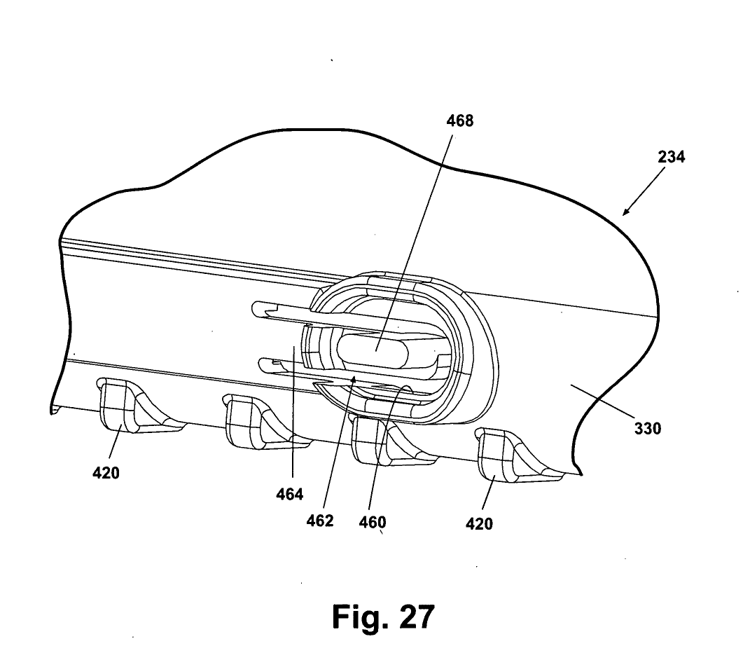

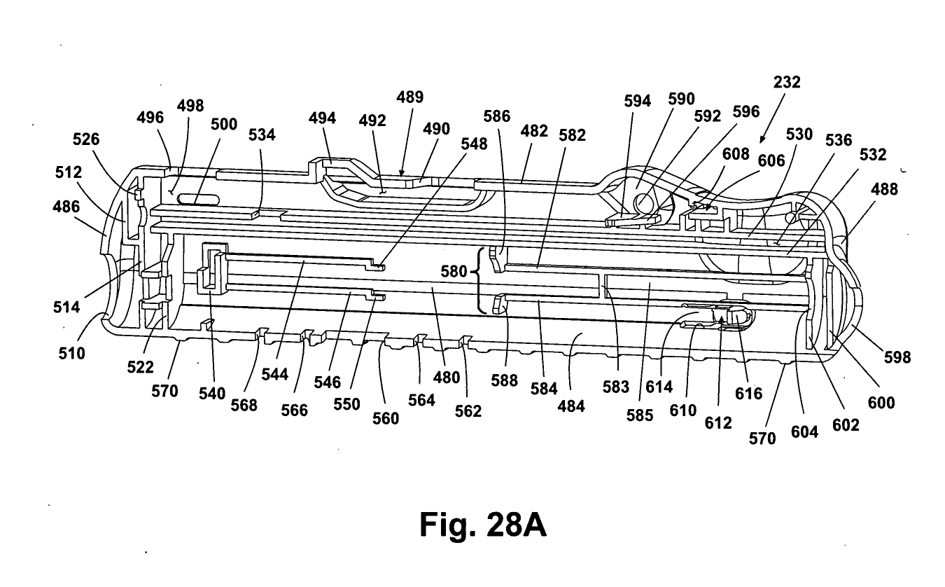

Referring also to FIG. 27, an elongated front trigger opening 460 penetrates the side wall 330 at a lower, distal region thereof, ventrally of the lower rib 434. Extending distally into the opening 460 from the proximal end thereof is a cantilevered release arm 462. The release arm 462 comprises a resilient, cantilever member 464 terminating in an inwardly-extending tooth 466. Extending outwardly from the cantilever member 464 in opposed disposition to the tooth 466 is a button boss 468.

Referring again to FIG. 26C, the distal wall 338 extends from the top wall 332 to the bottom wall 334 and has an irregular compound curvature. The distal wall 338 is inset with an irregularly-shaped nosepiece opening 448 in axial alignment with the nosepiece opening 454 in the inner wall 452.

Referring again to FIG. 26A, the bottom wall 334 extends from the distal wall 338 to the proximal wall 336 in generally parallel juxtaposition with the top wall 332. The bottom wall 334 is provided along its length with an array of regularly-spaced, laterally extending ribs 420. An elongated, rectilinear sample size selector slot 410 is inset in the bottom wall 334 proximally of the midpoint thereof. A pair of rectilinear distal slots 412, 414 is inset in the bottom wall 334 distally of the sample size selector slot 410. A pair of rectilinear proximal slots 416, 418 is inset in the bottom wall 334 proximally of the sample size selector slot 410. The spacing of the distal slots 412, 414 is equal to the spacing of the proximal slots 416, 418.

The side wall 330 and the top wall 332 transition toward the distal end of the housing shell 234 in a laterally outwardly-opening pivot receptacle 440 provided with a circular pivot aperture 442 extending therethrough. Extending inwardly from the side wall 330 intermediate the pivot receptacle 440 and the distal wall 338 is a horizontally disposed, plate-like distal cam block wedge 444 having a rearward facing inclined face 446.

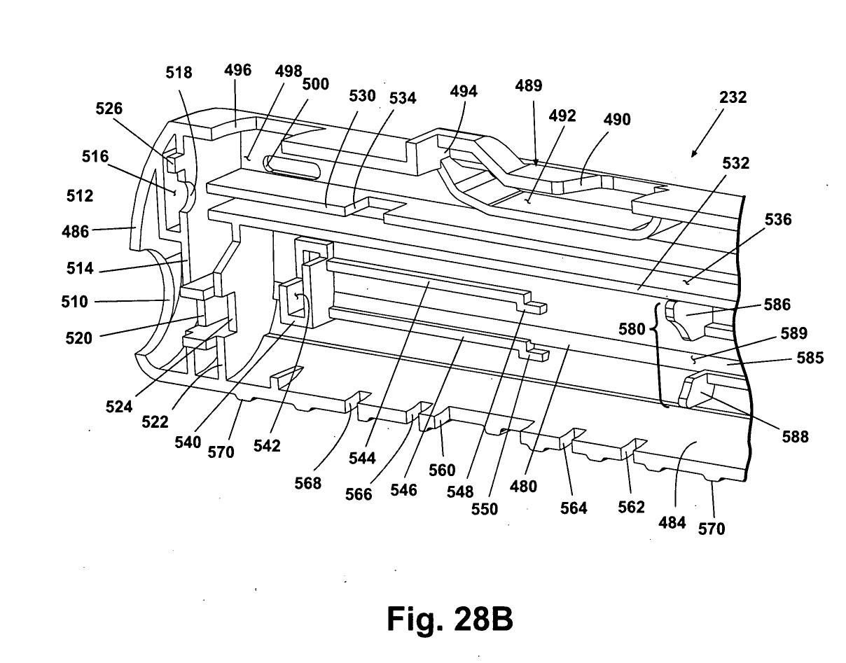

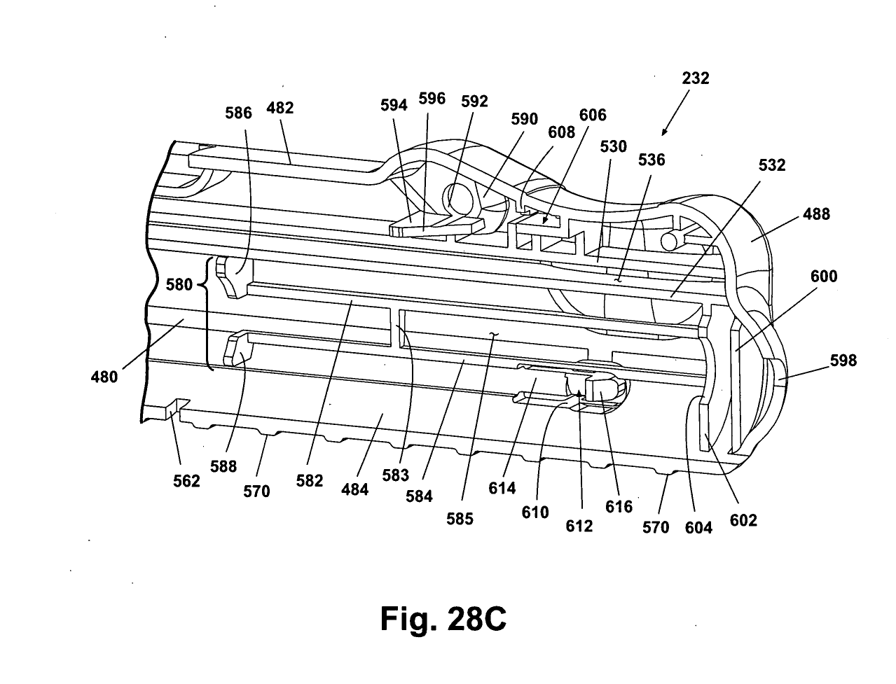

The left housing shell 232 is generally a mirror image, and has many of the same structural elements, of the right housing shell 234 arranged for cooperative registry of the structural elements in both shells 232, 234 to provide support and movement functionality to the assembled housing 230. FIGS. 28A-C and 29 illustrate the left housing shell 232. The left housing shell 232 is an irregularly-shaped, elongated body comprising an elongated side wall 480 joined to a top wall 482, a bottom wall 484, a proximal wall 486, and a distal wall 488. The walls 480-488 are contoured, and configured with openings, bosses, rails, and the like, for operational support of the elements comprising the biopsy gun 220. Referring specifically to FIG. 28B, the top wall 482 is provided with a swing arm opening 490 therethrough entering into a swing arm cage 489 comprising an elongated, somewhat curved swing arm chamber 492 terminating in a proximal end 494 inclined upwardly away from the swing arm opening 490. Proximal of the swing arm opening 490, the top wall 482 is provided with an upper latch opening 496 therethrough entering into a latch chamber 498. A latch slot 500 communicates with the latch chamber 498 through the side wall 480.

The proximal wall 486 curves somewhat outwardly from the top wall 482 to the bottom wall 484, and is inset with a semicircular trigger opening 510 at a lower portion thereof. Extending inwardly from the side wall 480, and depending from an upper portion of the proximal wall 486, is an abbreviated outer wall 512. Extending inwardly from the side wall 480, and from the top wall 482 to the bottom wall 484, parallel to the outer wall 512, is an intermediate wall 514. An upper portion of the intermediate wall 514 is joined to the outer wall 512 to form a narrow spring retainer chamber 516. The intermediate wall 514 is inset with a semicircular spring retainer opening 518 at an upper portion thereof in communication with the spring retainer chamber 516, and with a rectilinear intermediate trigger opening 520 at a lower portion thereof in axial alignment with the trigger opening 510. Extending inwardly from the intermediate wall 514 dorsally of the spring retainer opening 518 is a rectilinear retainer boss 526. Extending inwardly from the side wall 480, and upwardly from the bottom wall 484, parallel to the intermediate wall 514, is an inner wall 522. The inner wall 522 is inset with a rectilinear distal trigger opening 524 in axial alignment with the intermediate trigger opening 520 and the trigger opening 510.

Extending inwardly from the sidewall 480 generally parallel to the top wall 482, and from the intermediate wall 514 toward the distal wall 488, are an upper interior wall 530 and a lower interior wall 532 in parallel, spaced-apart juxtaposition. The upper interior wall 530 is inset with a rectilinear lower latch opening 534 therethrough at a proximal end thereof. The upper interior wall 530 and the lower interior wall 532 define an elongated shuttle slot 536 therebetween.

Extending inwardly from the side wall 480, distally of the inner wall 522 and ventrally of the lower interior wall 532, is a rectilinear latch plate support 540 defining a latch plate channel 542. Extending inwardly from the side wall 480 and distally from the latch plate support 540 are an upper spoon cannula carriage rail 544 and a lower spoon cannula carriage rail 546 in parallel, spaced-apart juxtaposition. The upper spoon cannula carriage rail 544 terminates in an upper stop 548. The lower spoon cannula carriage rail 546 terminates in a lower stop 550. The carriage rails 544, 546 are generally parallel to the upper interior wall 530 and the lower interior wall 532. A cutting cannula carriage cradle 580 comprises an upper cradle piece 586 and a lower cradle piece 588 extending inwardly from an intermediate location on the side wall 480 and defining an arcuate, inward-facing surface. The cradle pieces 586, 588 are separated by a slot 589.

Referring now to FIG. 28C, the upper interior wall 530 and the lower interior wall 532 extend distally to an intermediate wall 600 extending inwardly from the side wall 480 and upwardly from the bottom wall 484. An inner wall 602 extends inwardly from the side wall 480 and upwardly from the bottom wall 484 parallel to the intermediate wall 600. The inner wall 602 is inset with a nosepiece opening 604 therethrough. Referring again to FIG. 28B, an upper rib 582 and a lower rib 584 extend inwardly from the side wall 480 from the cutting cannula carriage cradle 580 to the inner wall 602 in parallel, spaced-apart juxtaposition, ventrally of and generally parallel to the lower interior wall 532 to define a slot 585. A stop rib 583 extends orthogonally from the upper rib 582 to the lower rib 584 intermediate the ends of the ribs 582, 584.

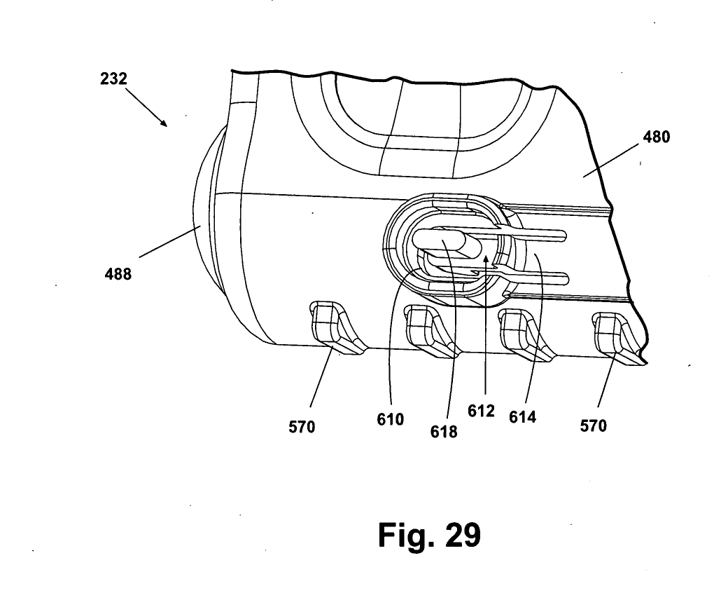

Referring also to FIG. 29, an elongated front trigger opening 610 penetrates the side wall 480 at a lower, distal region thereof, ventrally of the lower rib 584. Extending distally into the opening 610 from the proximal end thereof is a cantilevered release arm 612. The release arm 612 comprises a resilient, cantilever member 614 terminating in an inwardly-extending tooth 616. Extending outwardly from the cantilever member 614 in opposed disposition to the tooth 616 is a button boss 618.

Referring again to FIG. 28C, the distal wall 488 extends from the top wall 482 to the bottom wall 484 and has an irregular compound curvature. The distal wall 488 is inset with an irregularly-shaped nosepiece opening 598 in axial alignment with the nosepiece opening 604 in the inner wall 602.

Referring again to FIG. 28A, the bottom wall 484 extends from the distal wall 488 to the proximal wall 486 in generally parallel juxtaposition with the top wall 482. The bottom wall 484 is provided along its length with an array of regularly-spaced, laterally extending ribs 570. An elongated, rectilinear sample size selector slot 560 is inset in the bottom wall 484 proximally of the midpoint thereof. A pair of rectilinear distal slots 562, 564 is inset in the bottom wall 484 distally of the sample size selector slot 560. A pair of rectilinear proximal slots 566, 568 is inset in the bottom wall 484 proximally of the sample size selector slot 560. The spacing of the distal slots 562, 564 is equal to the spacing of the proximal slots 566, 568.

The side wall 480 and the top wall 482 transition toward the distal end of the housing shell 232 in a laterally outwardly-opening pivot receptacle 590 provided with a circular pivot aperture 592 extending therethrough. Extending inwardly from the pivot receptacle 590 immediately below the pivot aperture 592 is a horizontally disposed, plate-like proximal cam block wedge 594 having a forward facing inclined face 596. Distally of the pivot receptacle 590 is a rectilinear cam spring housing 606 having a stop wall 608 depending from the top wall 482. The stop wall 608 is separated from the cam spring housing 606 so that the cam spring housing 606 opens proximally toward the pivot receptacle 590.

The housing shells 232, 234 are provided with a suitable number of connecting elements, such as snapfit retainers, post and seat structures, threaded fastener apertures and seats, and the like, for fixedly interconnecting the housing shells 232, 234 to form the housing 230.

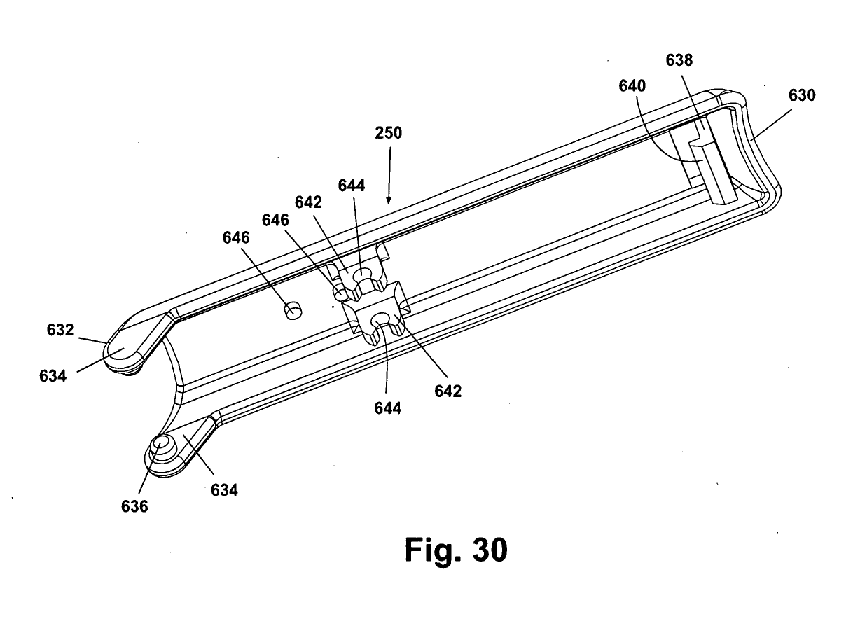

Referring now to FIG. 30, the handle 250 is an elongated, beam-like member having a proximal end 630 and a distal end 632. The distal end 632 terminates in a pair of parallel, spaced-apart, inclined pivot extensions 634, each of which terminates in an inwardly extending pivot boss 636. The pivot extensions 634 and pivot bosses 636 are adapted for receipt in the pivot receptacles 440, 590 and the pivot apertures 442, 592, respectively, with the housing shells 232, 234 in an assembled configuration. The pivot receptacles 440, 590 are adapted for pivotal movement of the handle 250 about an axis coaxial with the pivot apertures 442, 592.

A latch extension 638 extends orthogonally from the underside of the handle 250 at the proximal end 630 thereof, terminating in a hook 640 extending toward the distal end 632. A pair of parallel, spaced-apart swing arm supports 642 extends from the underside of the handle 250 intermediate the proximal end 630 and the distal end 632. The swing arm supports 642 are provided with pivot openings 644 defining a rotational axis orthogonal to the longitudinal axis of the handle 250. Intermediate the swing arm supports 642 and the distal end 632 is a pair of cylindrical spring mounting bosses 646 extending from the underside of the handle 250.

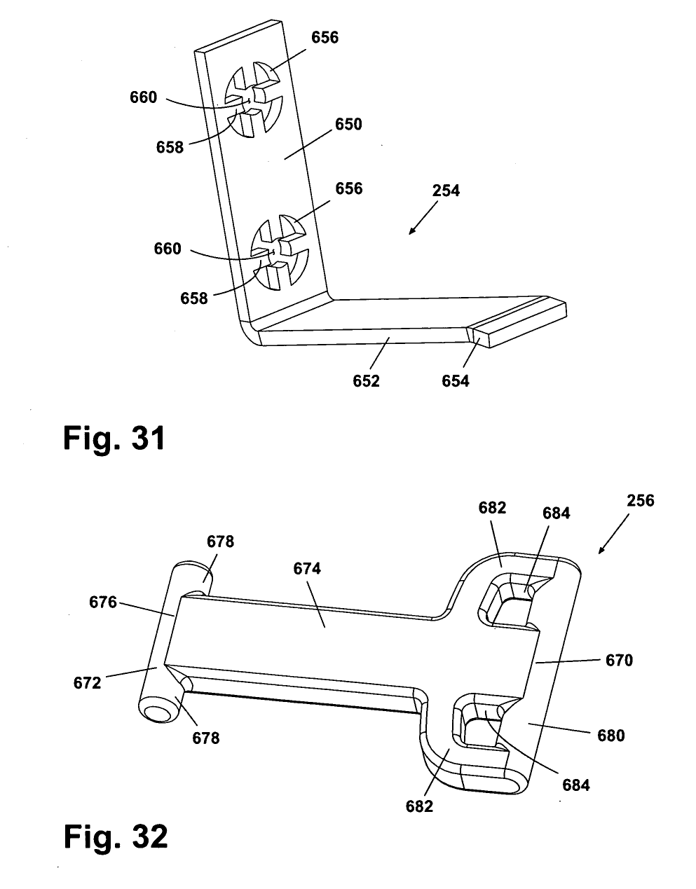

Referring now to FIG. 31, the spring 254 is a resilient, generally L-shaped member comprising a mounting arm 650 and a contact arm 652 extending generally orthogonally therefrom. The contact arm 652 terminates in a flange 654 inclined somewhat away from the mounting arm 650. A spaced pair of mounting openings 656 having a generally circular configuration extends through the mounting arm 650. A plurality, illustrated in FIG. 31 as numbering four, of mounting fingers 658 extends in cantilevered fashion radially inwardly from the perimeter of each mounting opening 656 to terminate in spaced disposition to define a center aperture 660 coaxial with the center of the mounting opening 656. The ends of each finger 658 can be provided with a curved tip to define a circular center aperture 660. The size of the center apertures 660 is adapted for insertion of the spring mounting bosses 646 therethrough for fixed frictional attachment of the spring 254 to the handle 250. The spring 254 is fabricated of a suitable material, such as steel or high strength plastic, having sufficient strength and resiliency for the purposes described herein.

As illustrated in FIG. 32, the swing arm 256 is a generally flattened, somewhat T-shaped member having a pivot end 670 and a sliding end 672 interconnected by a center beam 674. The sliding end 672 comprises a cylindrically shaped sliding rod 676 orthogonal to the longitudinal axis of the center beam 674 and extending laterally away from the center beam 674 to define a pair of opposed end bosses 678. The pivot end 670 comprises a cylindrically shaped pivot rod 680 orthogonal to the longitudinal axis of the center beam 674 and extending laterally away from the center beam 674, parallel to the sliding rod 676. A pair of curved buttresses 682 extend from the ends of the pivot rod 680 to join the center beam 674 at an intermediate region thereof, to define with the pivot rod 680 and the center beam 674 a pair of spaced-apart openings 684. The sections of the pivot rod 680 adjacent the openings 684 are adapted for insertion into the pivot openings 644 of the swing arm supports 642 of the handle 250 for pivotal mounting of the swing arm 256 to the handle 250. Preferably, the swing arm supports 642 are adapted so that the pivot rod 680 is snapped into the pivot openings 644.

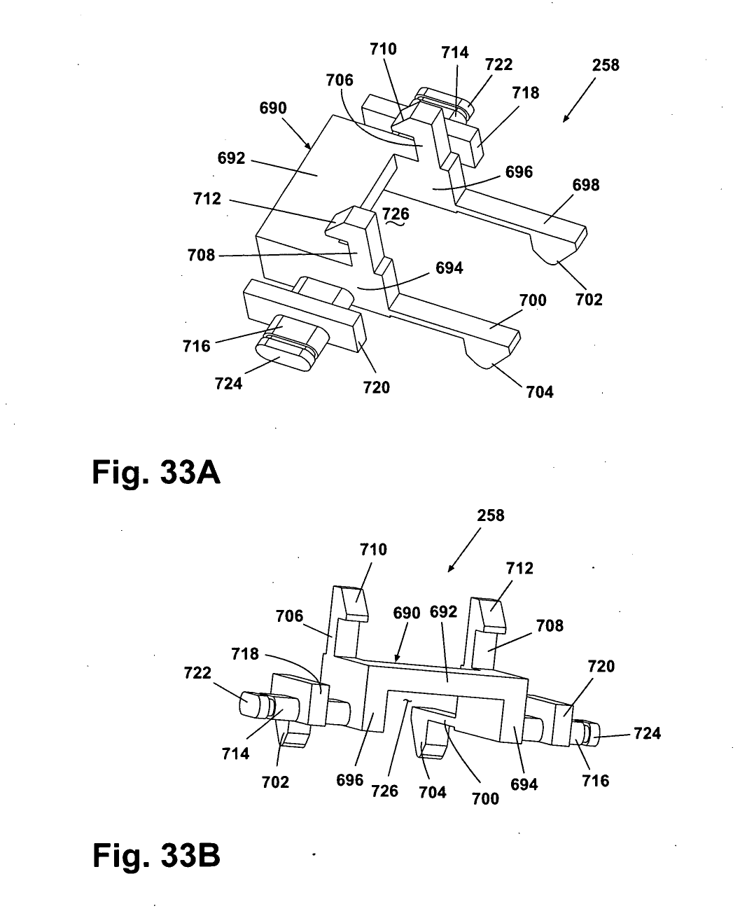

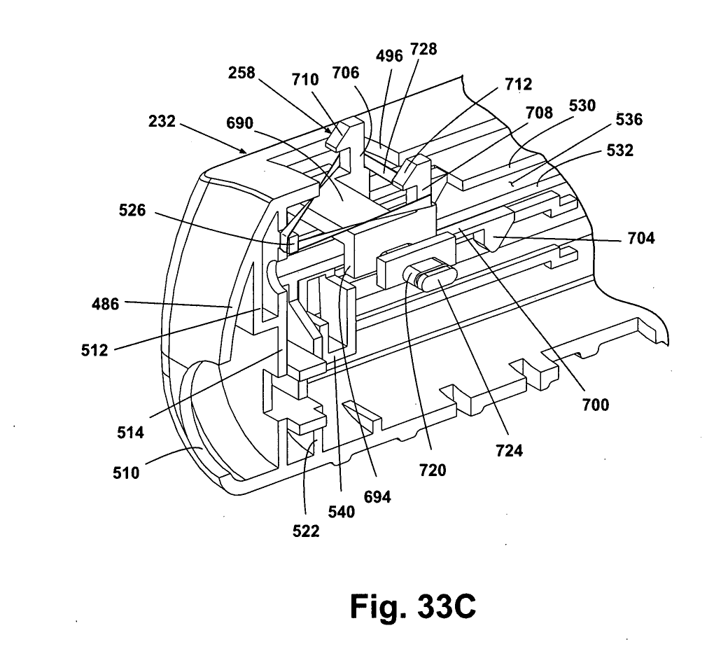

Referring now to FIGS. 33A-C, the latch 258 is a carriage-like body comprising a rectilinear frame portion 690 having a center wall 692 joining a pair of orthogonally-depending, spaced-apart sidewalls 694, 696. The center wall 692 and side walls 694, 696 define a rectilinear shuttle cavity 726. Extending away from the side wall 694 in cantilever fashion and coplanar therewith is a flex arm 700 terminating away from the side wall 694 in a downwardly depending tooth 704. Parallel to the flex arm 700, extending away from the side wall 696 in cantilever fashion and coplanar therewith is a flex arm 698 terminating away from the side wall 696 in a downwardly depending tooth 702. Extending away from the center wall 692 in cantilever fashion coplanar with the side wall 694 is a latch arm 708 terminating away from the center wall 692 in a hook 712 extending above the center wall 692. Extending away from the center wall 692 in cantilever fashion coplanar with the side wall 696 is a latch arm 706 terminating away from the center wall 692 in a hook 710 extending above the center wall 692. A flattened lateral beam 714 extends outwardly away from the side wall 696 to terminate in a button mount 722. An elongated flange plate 718 intersects the lateral beam 714 parallel to and spaced away from the side wall 696. A flattened lateral beam 716 extends outwardly away from the side wall 694 to terminate in a button mount 724. An elongated flange plate 720 intersects the lateral beam 716 parallel to and spaced away from the side wall 694. The lateral beams 714, 716 are coaxially aligned.

As illustrated in FIG. 33C, the latch 258 is provided with an elastic band 728 adapted to encircle the latch arms 706, 708 and the retainer boss 526 when the latch 258 is installed in the housing 230, as hereinafter described.

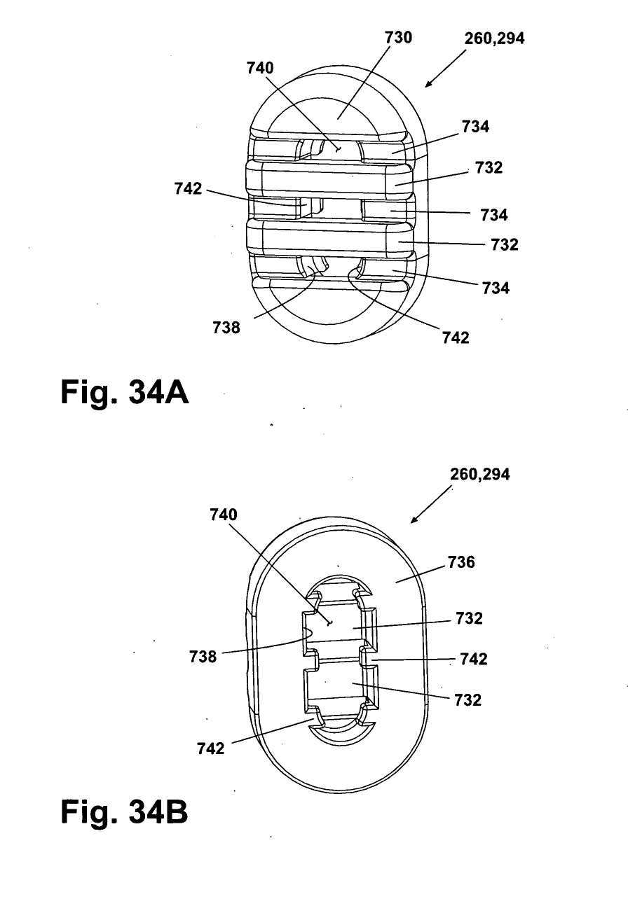

Referring now to FIGS. 34A-B, the button 260, 294 is a somewhat oval-shaped, rounded body having an outer surface 730 and an opposed inner surface 736. The outer surface 730 is divided into ribs 732 by an array of regularly-spaced slots 734 oriented orthogonal to the longitudinal axis of the button 260, 294. A somewhat oval-shaped perimeter wall 738 depends from the inner surface 736 to the ribs 732 to define a cavity 740. The cavity 740 is adapted for slidable insertion of a button mount 722, 724, or a button boss 318, 468, therein. A plurality of inwardly extending pegs 742 is spaced around the perimeter wall 738 to facilitate the fixed attachment of the button 260 to the button mount 722, 724, and the button boss 318, 468.

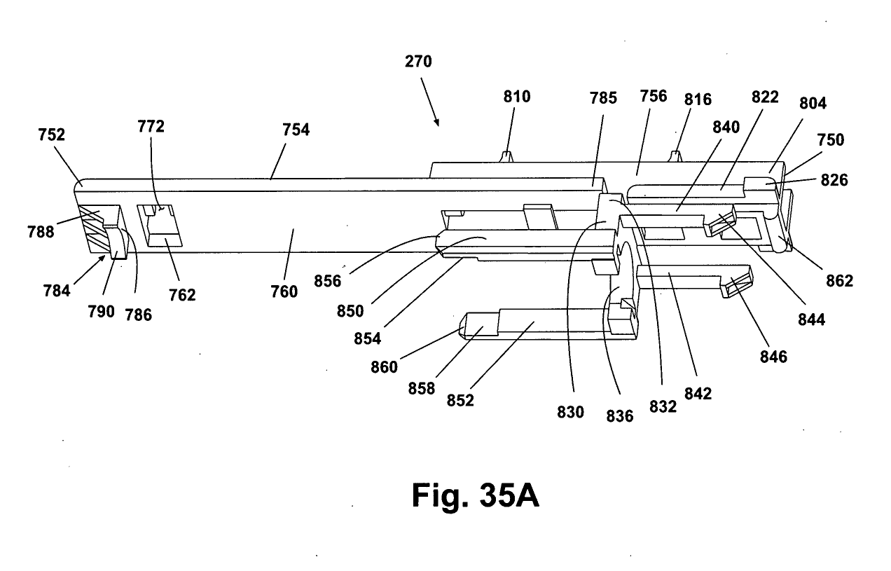

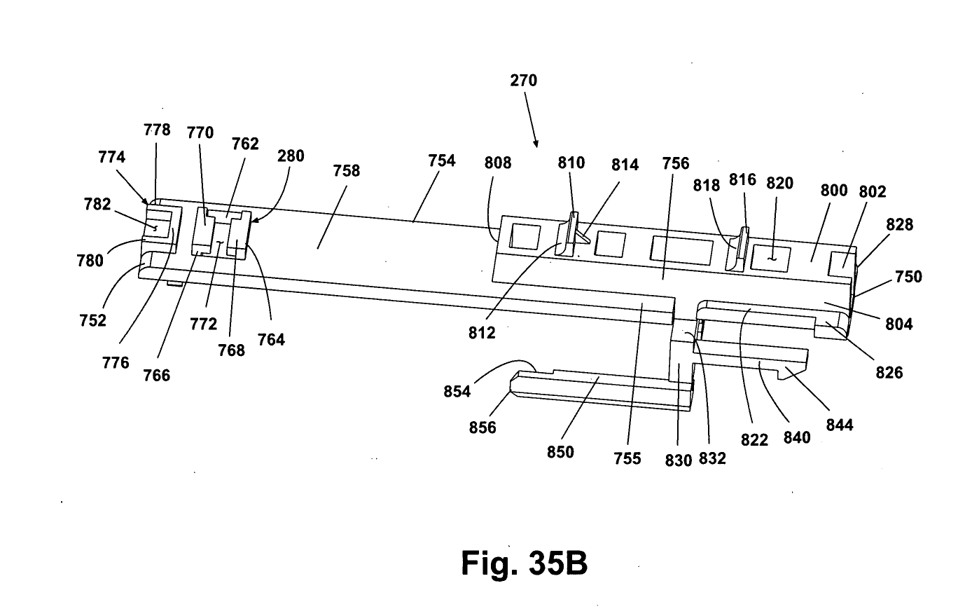

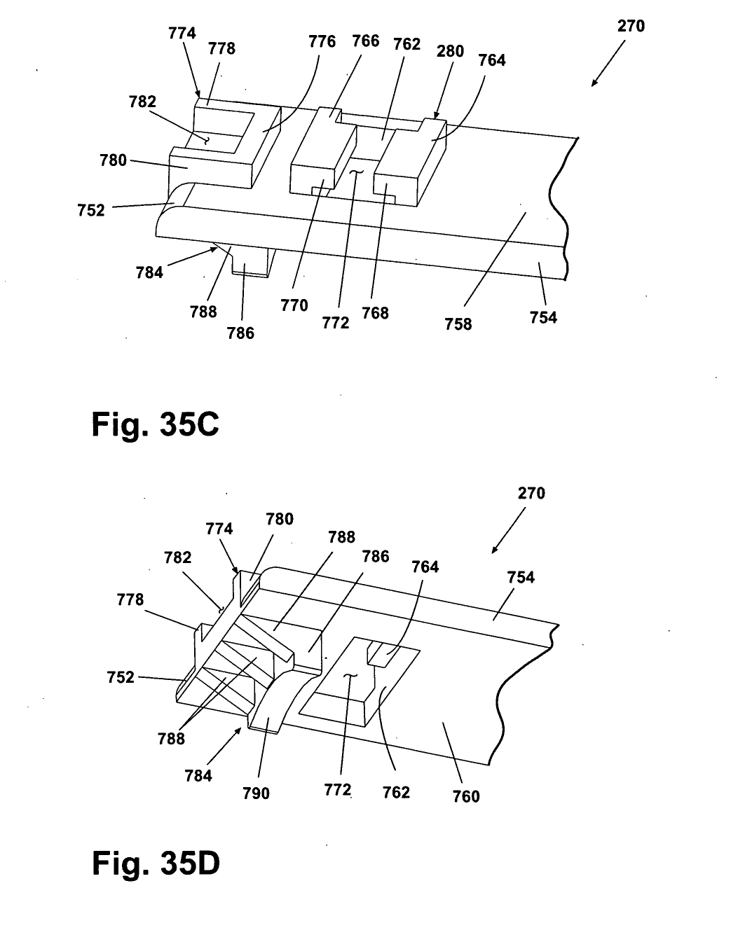

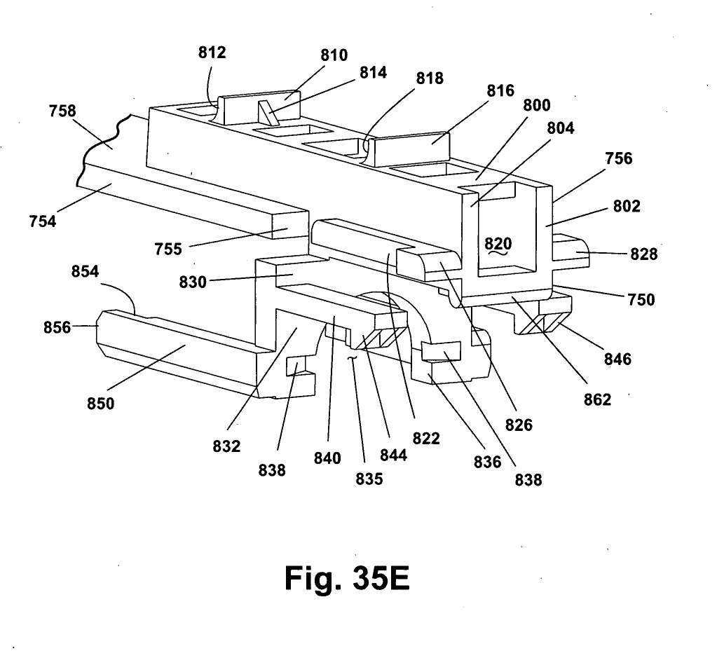

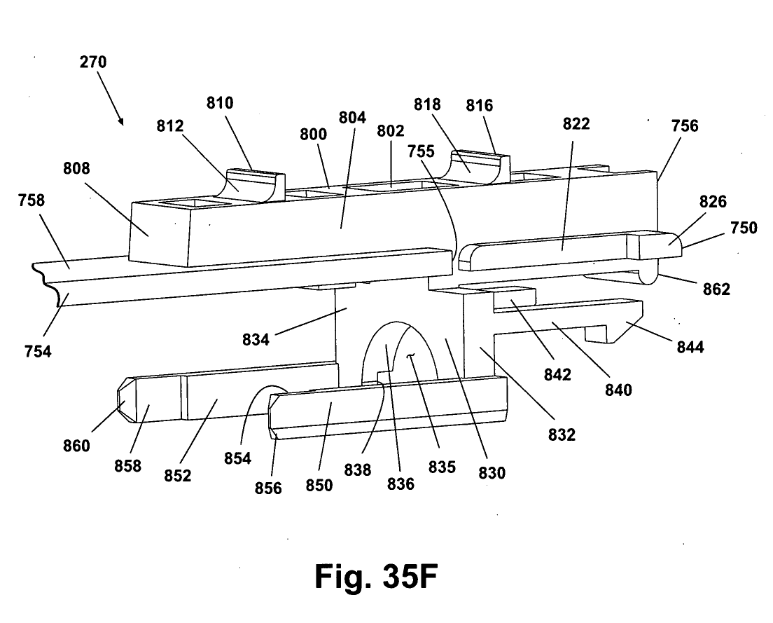

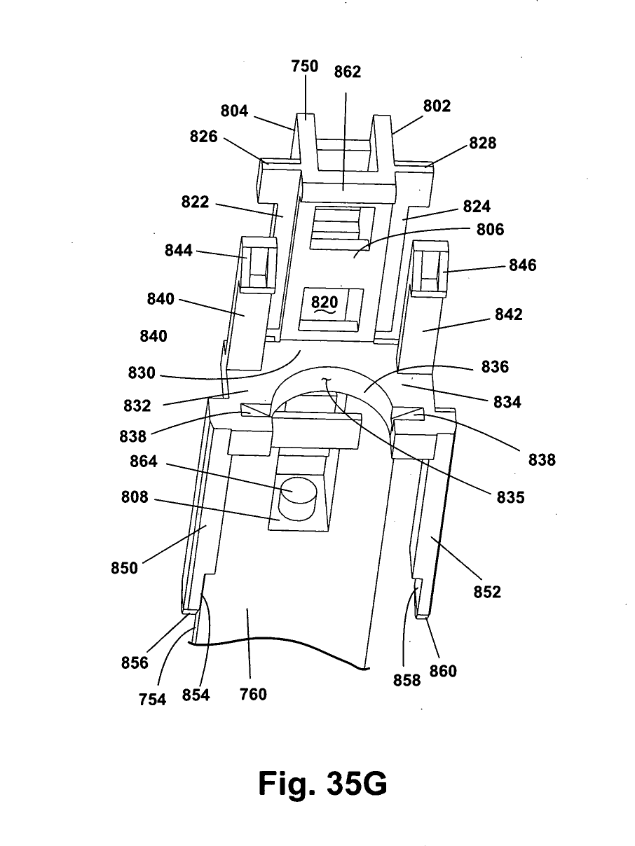

As illustrated in FIGS. 35A-G, the shuttle 270 is an elongated carriage-like body having a proximal end 750 and a distal end 752, and comprising a plate portion 754 and a box portion 756. The plate portion 754 extends from the distal end 752 into cooperative registry with the box portion 756, terminating in a plate proximal end 755 at approximately the mid-line of the box portion 756. The box portion 756 extends from the plate portion 754 to the proximal end 750. The plate portion 754 is an elongated, plate-like member having an upper surface 758 and a lower surface 760. A rectilinear opening 762 extends through the plate portion 754 near the distal end 752.

Referring to FIG. 35C, a cam block retainer 280 comprises an inner wall 764 extending laterally along the opening 762 orthogonally away from the upper surface 758, and an outer wall 766 extending laterally along the opening 762 orthogonally away from the upper surface 758 parallel to the inner wall 764. An inner flange 768 extends orthogonally along the top of the inner wall 764 over the opening 762, and an outer flange 770 extends orthogonally along the top of the outer wall 766 over the opening 762 coplanar with the inner flange 768 in spaced disposition therewith to define a gap 772 therebetween.

An end box 774 extends distally of the cam block retainer 280 and comprises an inner wall 776 extending orthogonally away from the upper surface 758, and a pair of side walls 778, 780 extending orthogonally away from the upper surface 758 and the inner wall 776 in parallel, spaced-apart juxtaposition. The walls 776-780 define a rectilinear chamber 782 opening toward the distal end 752. Referring to FIG. 35D, depending from the lower surface 760 intermediate the distal end 752 and the opening 762 is a cradle 784 comprising a cradle wall 786 terminating in an arcuate surface 790 opening away from the lower surface 760. The cradle wall 786 is reinforced with a plurality of triangular braces 788 between the cradle wall 786 and the distal end 752. The distal end 752 terminates in a rounded edge depending from the upper surface 758.

Referring to FIGS. 35E and F, the box portion 756 is an elongated, rectilinear structure comprising a top wall 800 parallel to and spaced away from the upper surface 758 of the plate portion 754, and a bottom wall 806 parallel to and spaced away from the top wall 800 parallel to the plate portion 754. A pair of parallel, spaced-apart side walls 802, 804 depends orthogonally from the top wall 800 for connection, in part, with the plate portion 754 and, in part, with the bottom wall 806. An inner end wall 808 extends orthogonally away from the upper surface 758 of the plate portion 754 to join the side walls 802, 804 to enclose a distal end of the box portion 756. The walls 800-808 define an elongated, rectilinear chamber 820. As illustrated in FIG. 24G, a cylindrical spring boss 864 extends orthogonally from the inner end wall 808 into the chamber 820.

Referring again to FIGS. 35E and F, an inner bearing wall 810 extends orthogonally away from the top wall 800 adjacent the inner end wall 808. The inner bearing wall 810 is provided with an arcuate surface 812 on the distal side of the wall 810. A triangular brace 814 extends from the proximal side of the inner bearing wall 810 to the top wall 800. An outer bearing wall 816 extends orthogonally away from the top wall 800 intermediate the inner bearing wall 810 and the proximal end 750. The outer bearing wall 816 is provided with an arcuate surface 818 on the distal side of the wall 816.

An elongated side rib 822 extends laterally away from the side wall 804 coplanar with the plate portion 754. An end boss 826 extends laterally away from the proximal end of the side rib 822. An elongated side rib 824 extends laterally away from the side wall 802 coplanar with the plate portion 754. An end boss 828 extends laterally away from the proximal end of the side rib 824. The proximal end of each end boss 826, 828 is rounded. Depending from the proximal end of the bottom wall 806 is a semicylindrical end boss 862 disposed transversely of the longitudinal axis of the shuttle 270.

Referring to FIGS. 35E-G, depending orthogonally from the bottom wall 806 proximally of the plate proximal end 755 is an intermediate wall 830 inset with a cradle opening 835 opening ventrally away from the box portion 756 and defined by an arcuate surface 836. The lateral sides of the cradle 835 define a pair of downwardly-depending wings 832, 834. The proximal side of each wing 832, 834 is inset with a chamfered notch 838 inclined inwardly toward the cradle opening 835. Extending proximally from each wing 832, 834 parallel to the side walls 802, 804, respectively, is a cantilevered flex arm 840, 842, respectively, terminating proximally in a downwardly-depending hook 844, 846. Each flex arm 840, 842, terminates somewhat distally of the adjacent end boss 826, 828. Extending distally from a lower lateral portion of each wing 832, 834, parallel to the side walls 802, 804 is a cantilevered lower rail 850, 852. The lower rail 850 terminates in an inset end portion 854 having a tapered tip 856. The lower rail 852 terminates in an inset end portion 858 having a tapered tip 860.

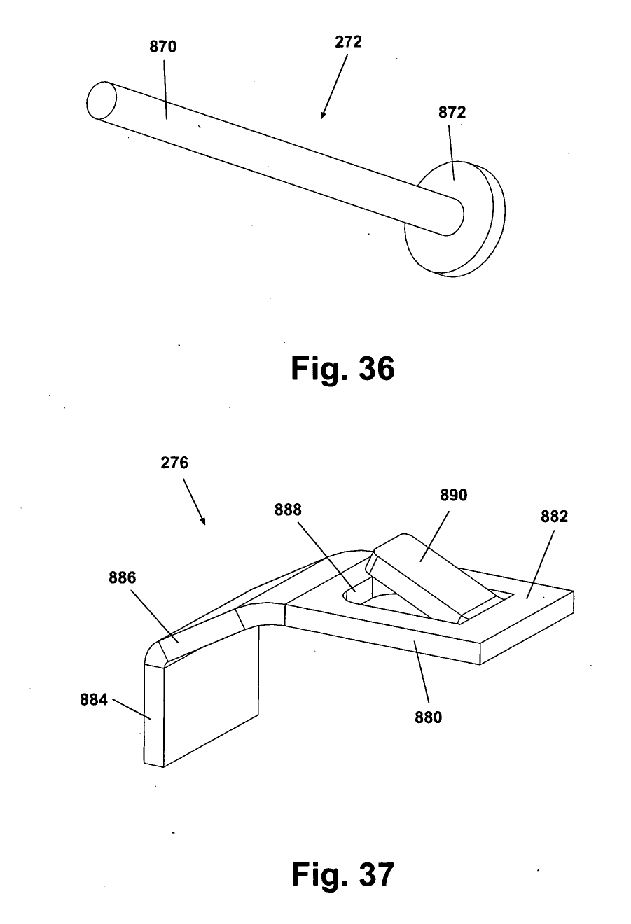

As illustrated in FIG. 36, the spring retainer 272 is an elongated, somewhat nail-shaped member comprising a cylindrical rod 870 terminating at one end in a circular flange 872 coaxial with the rod 870. The rod 870 is adapted for slidable receipt in the spring retainer openings 368, 518 with the flange 872 received in the spring retainer chambers 366, 516.

Referring to FIG. 37, the cam spring 276 is a somewhat L-shaped member having an upper leg 880 and a lower leg 884 angled away from the upper leg 880, the upper leg 880 and the lower leg 884 being interconnected by an inclined member 886. The upper leg 880 has an upper surface 882. A generally rectilinear opening 888 extends through the upper leg 880. A finger 890 extends in cantilevered fashion into the opening 888 toward the inclined member 886, and is inclined above the upper surface 882 to form a somewhat hook-like structure. The cam spring 276 is fabricated of a suitable material, such as steel or high strength plastic, having sufficient strength and resiliency for the purposes described herein.

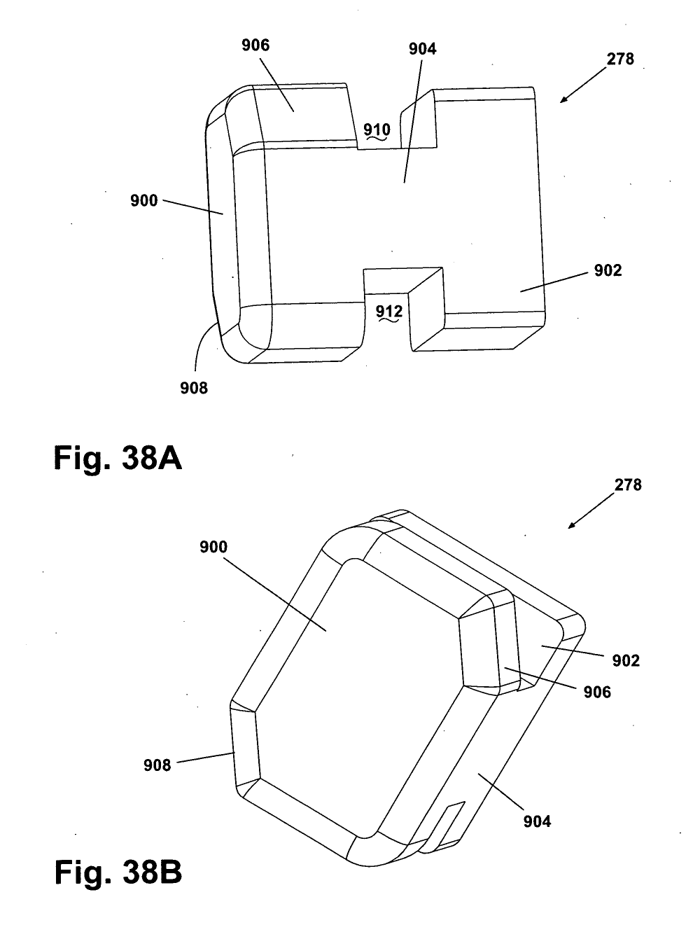

As illustrated in FIGS. 38A-B, the cam block 278 is a generally rectilinear body comprising an upper block 900 and a lower block 902 interconnected by a center beam 904. The upper block 900 is provided with a pair of diagonally juxtaposed inclined faces 906, 908 interrupting the rectilinear perimeter of the upper block 900. Laterally of the center beam 904 is a pair of parallel channelways 910, 912 separating the upper block 900 from the lower block 902. The cam block 278 is adapted for slidable receipt in the cam block retainer 280 with the beam 904 received in the gap 772 and the upper block 900 supported dorsally of the inner flange 768 and the outer flange 770.

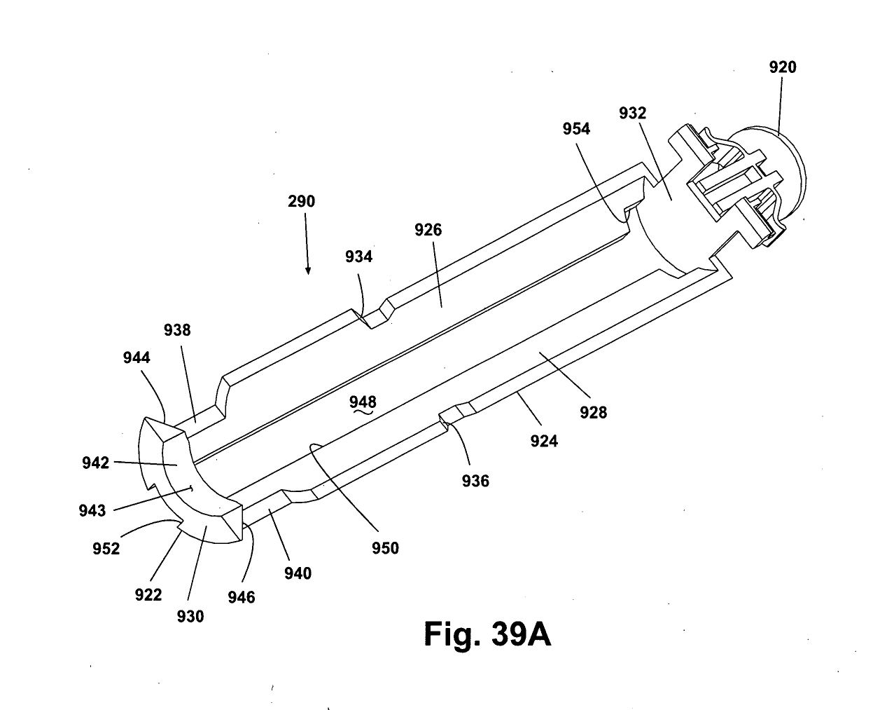

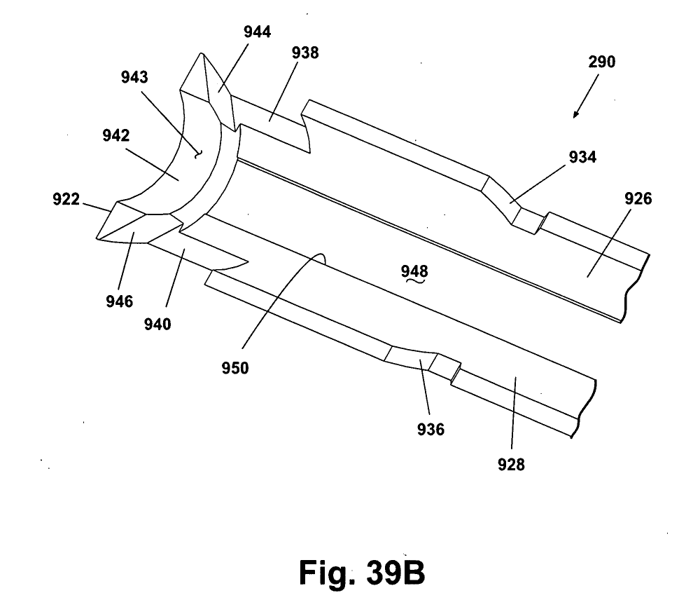

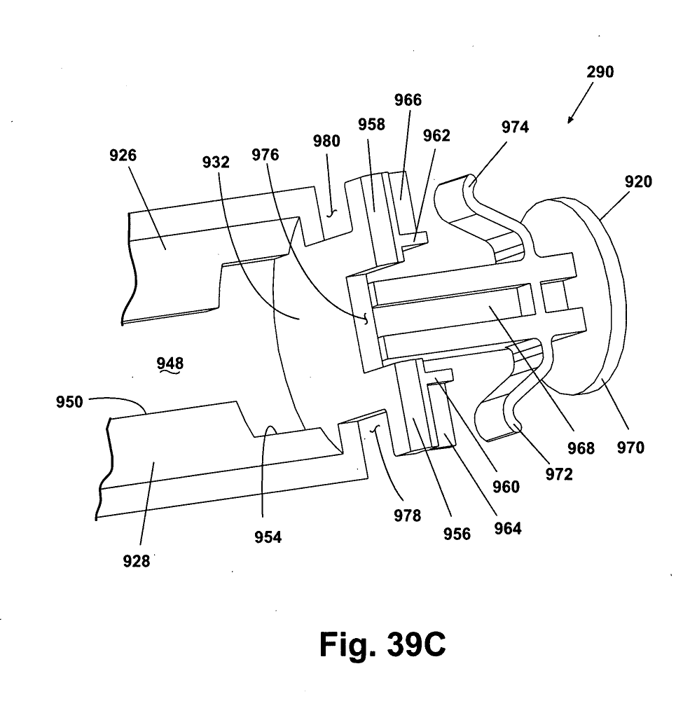

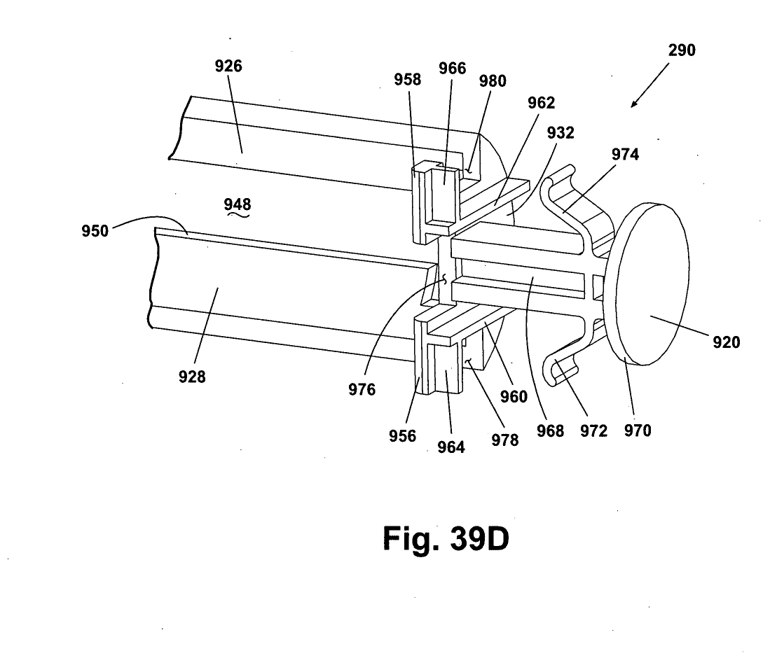

Referring now to FIGS. 39A-D, the firing cage 290 is an elongated member having a proximal end 920 and a distal end 922 joined by a center portion 924. The center portion 924 comprises a pair of parallel, spaced-apart side rails 926, 928 having a semicircular cross-section and extending ventrally between a distal end wall 930 and a proximal end wall 932. The side rails 926, 928 defined a semicylindrical, upwardly-opening channelway 948 between the distal end wall 930 and the proximal end wall 932. Each rail 926, 928 is inset with an upwardly opening center notch 934, 936, respectively, intermediate the end walls 930, 932, and an upwardly-opening end notch 938, 940, respectively, adjacent the end wall 930. The spacing of the rails 926, 928 defines a slot 950 extending longitudinally between the end walls 930, 932. The proximal end of the slot 950 expands laterally to define a rectilinear expanded slot 954.

Referring to FIG. 39B, the distal end wall 930 is a somewhat arch-shaped body extending orthogonally from the side rails 926, 928, and having an arcuate surface 942 opening dorsally to define a cradle opening 943. The distal end wall 930 is also provided with an end wall slot 952 extending therethrough coextensive with the slot 950. A pair of outwardly chamfered inclined faces 944, 946 abuts the ended notches 938, 940, respectively. Referring to FIG. 39C, the proximal end wall 932 is a somewhat T-shaped body inset with an upper notch 976 opening dorsally, and a pair of side notches 978, 980 opening laterally to define a pair of L-shaped flanges 956, 958, respectively, along a dorsal portion of the proximal end wall 934. Referring also to FIG. 39D, the proximal side of the proximal end wall 932 is provided with a pair of vertical braces 960, 962 and horizontal braces 964, 966 for strengthening the flanges 956, 958. Extending orthogonally away from the proximal side of the proximal end wall 932 is a somewhat I-beam shaped beam 968 terminating in a circular, plate-like plunger 970. Extending laterally from the beam 968 adjacent the plunger 970, parallel to the flanges 956, 958, is a pair of ogee-shaped spring arms 972, 974.

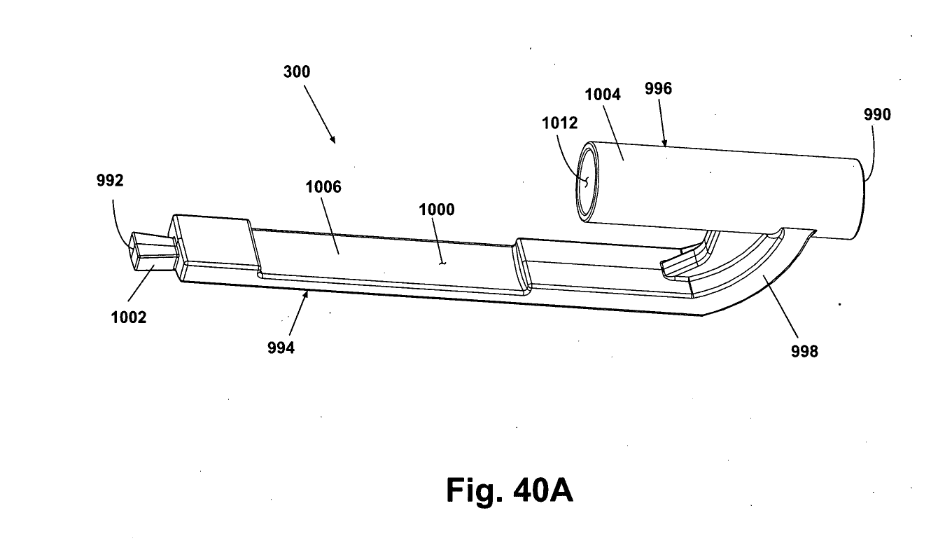

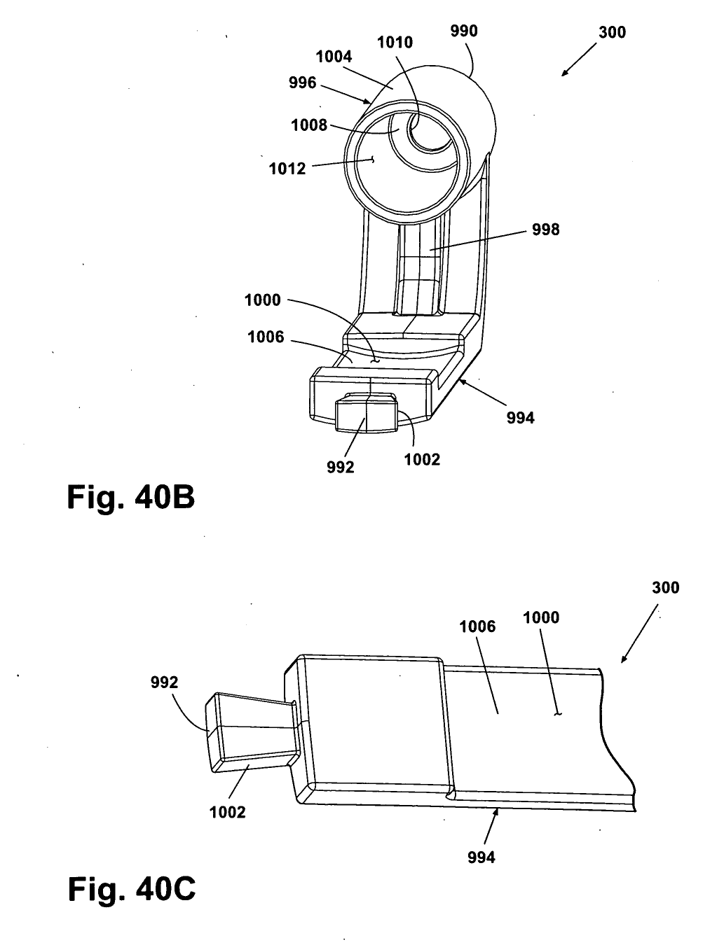

Referring now to FIGS. 40A-C, the nosepiece 300 is an elongated member having a distal end 990 and a proximal end 992, and comprising an elongated beam 994 interconnected in parallel, spaced-apart disposition with an elongated cylindrical collar 996 through a support member 998. The beam 994 is a generally strap-like member inset along an intermediate portion thereof with an upwardly-opening elongated mortise 1000 having an arcuate surface 1006, and terminating at the proximal end 992 in a dovetail 1002. The collar 996 comprises an annular wall 1004 terminating at the distal end 990 in a circular end wall 1008 having an aperture 1010 extending coaxially therethrough, and defining a cylindrical chamber 1012.

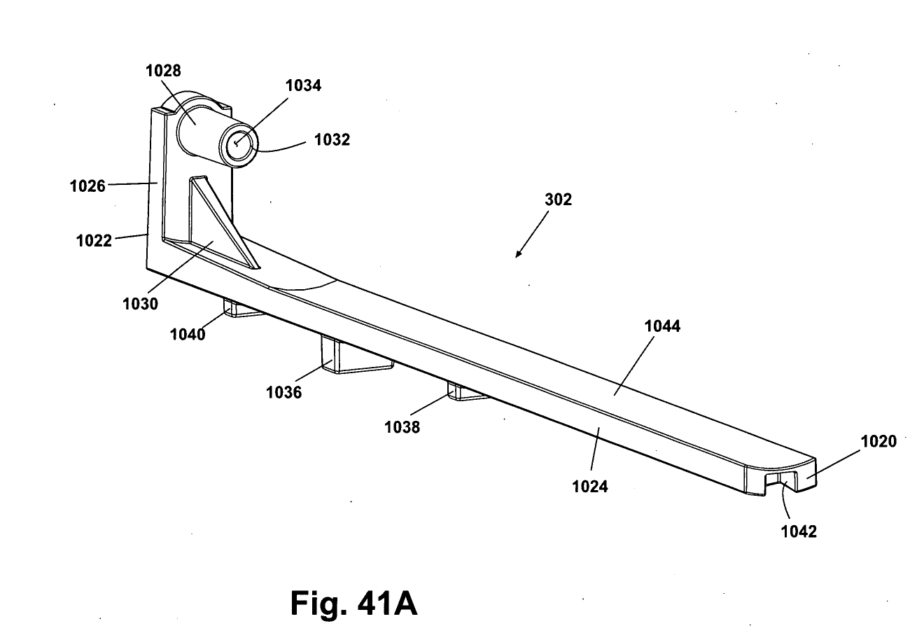

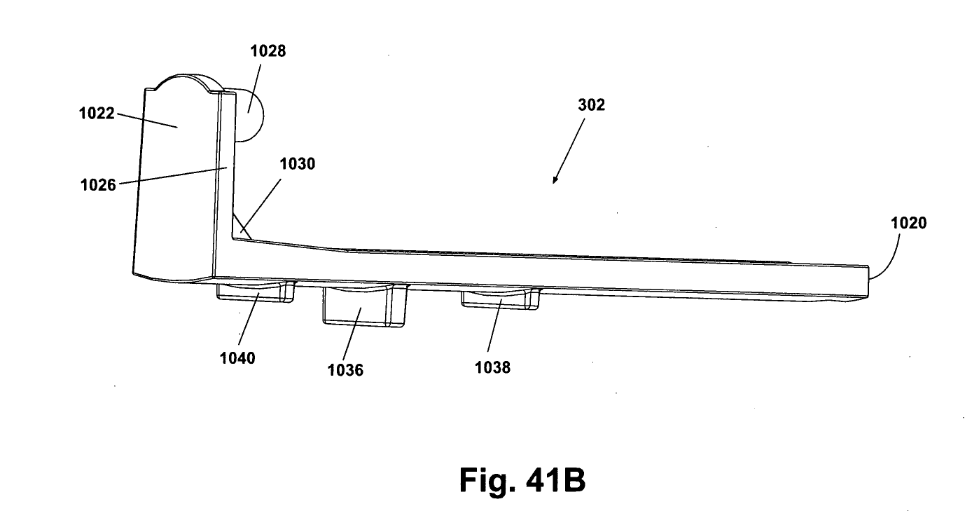

Referring now to FIGS. 41A-B, the adjustment member 302 is an elongated member having a distal end 1020 and a proximal end 1022, and comprising an elongated beam 1024 terminating at the proximal end 1022 in an orthogonally-disposed end wall 1026. The beam 1024 is a generally strap-like member having a cross-section approximating the cross-section of the beam 994, and a dorsal arcuate surface 1044 along its full length. The distal end 1020 is provided with a dovetail cutout 1042 adapted for slidable receipt of the dovetail 1002. The ventral surface of the beam 1024 is provided with a wall-like adjustment lever 1036 extending laterally across the beam 1024 orthogonal to the ventral surface. A wall-like forward stop 1038 extends laterally across the beam 1024 orthogonal to the ventral surface and parallel to the adjustment lever 1036, distally thereof. A wall-like rearward stop 1040 extends laterally across the beam 1024 orthogonal to the ventral surface and parallel to the adjustment lever 1036, proximally thereof.

The end wall 1026 terminates at a dorsal end in a cylindrical stylet seat 1028 comprising an annular wall 1032 defining a cylindrical chamber 1034 opening away from the end wall 1026 toward the distal end 1020. The chamber 1034 is adapted for slidable receipt of the proximal end of the stylet 20 therein. A triangular brace 1030 extends between the end wall 1026 and the beam 1024.

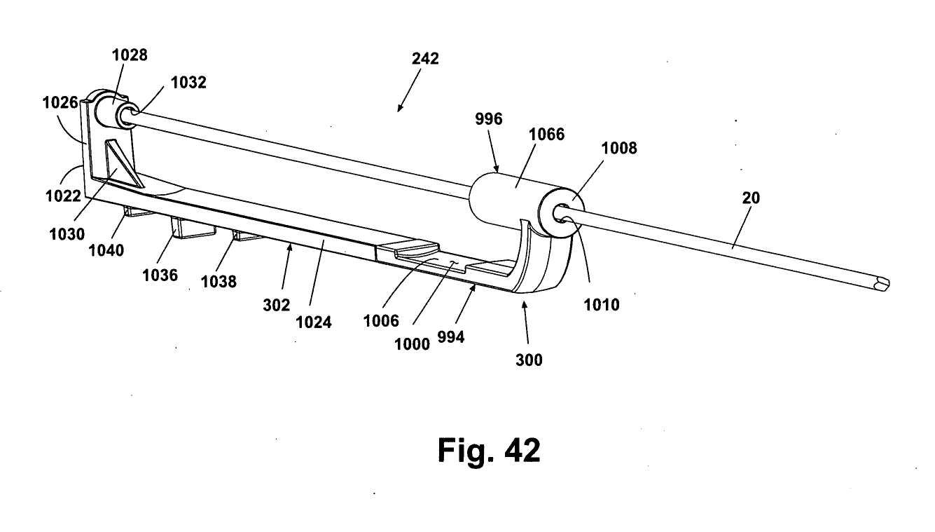

As illustrated in FIG. 42, the nosepiece 300 is joined to the adjustment member 302 by insertion of the dovetail 1002 into the dovetail cutout 1042 to form the sample size control assembly 142. The proximal end of the stylet 20 is held in the chamber 1034 so that the stylet 20 extends into the chamber 1012 and through the aperture 1010.

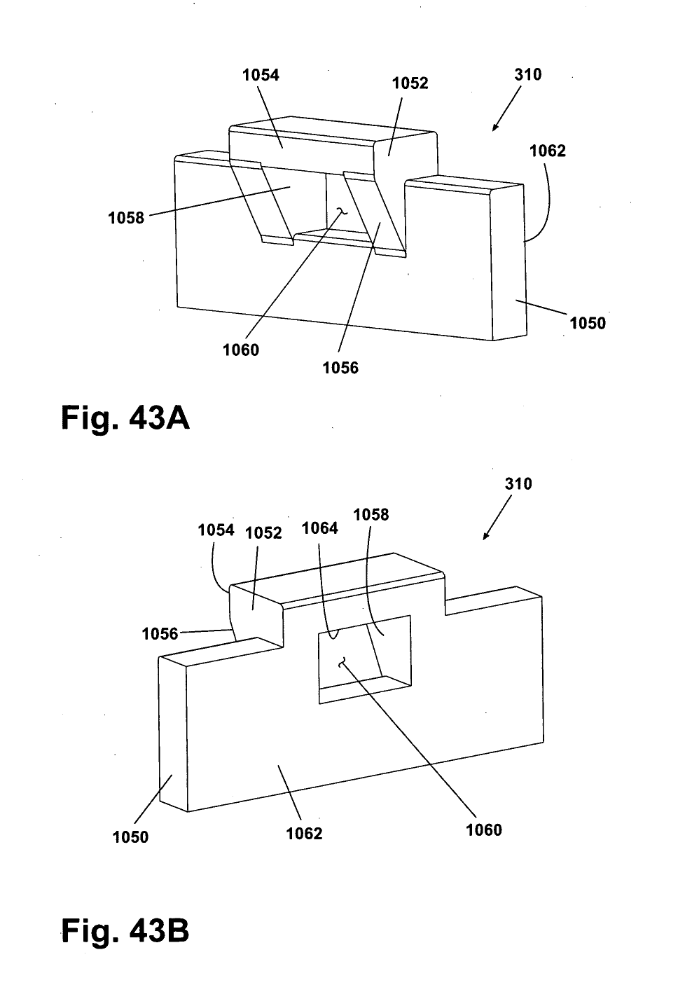

Referring now to FIGS. 43A-B, the latch plate 310 is a wall-like body comprising a plate portion 1050 and a crown portion 1052. The plate portion 1050 has a planar surface 1062 along a distal side thereof. The crown portion 1052 comprises a top wall 1054 disposed proximal and orthogonal to the plate portion 1050 and attached to the plate portion 1050 through a pair of triangular braces 1056, 1058 orthogonally intersecting the plate portion 1050 at an upper portion thereof to define a rectilinear opening 1060 therethrough. The top wall 1054 terminates in a top edge 1064 along an upper portion of the opening 1060.

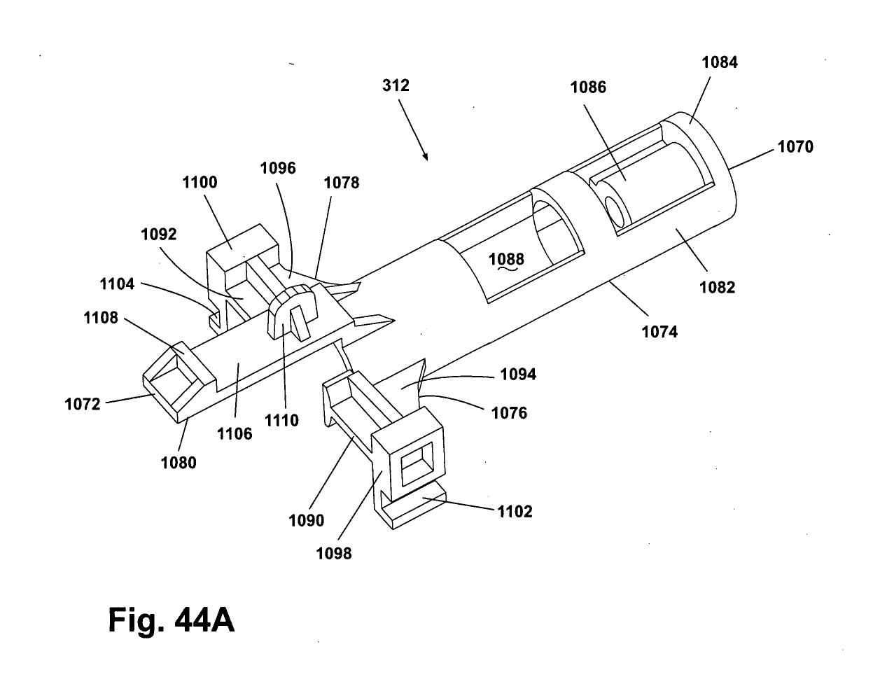

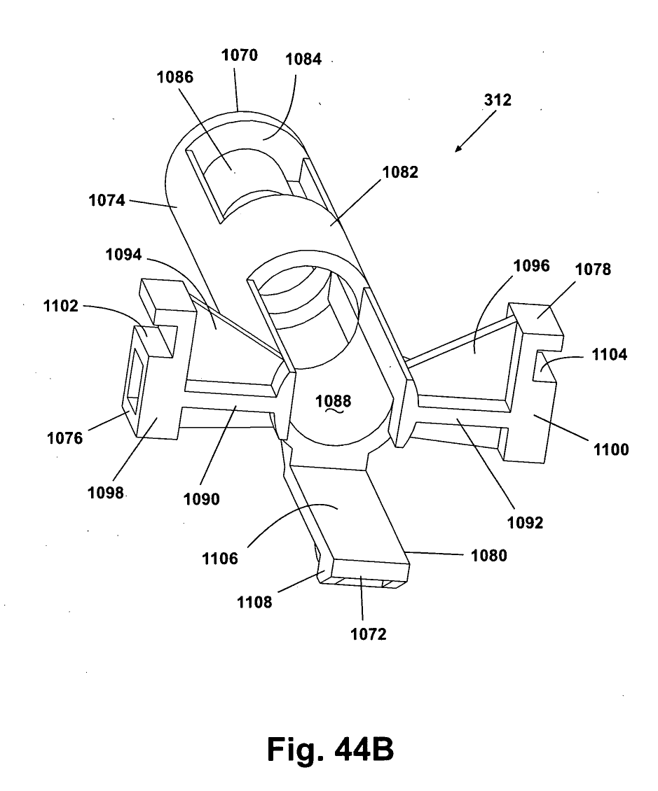

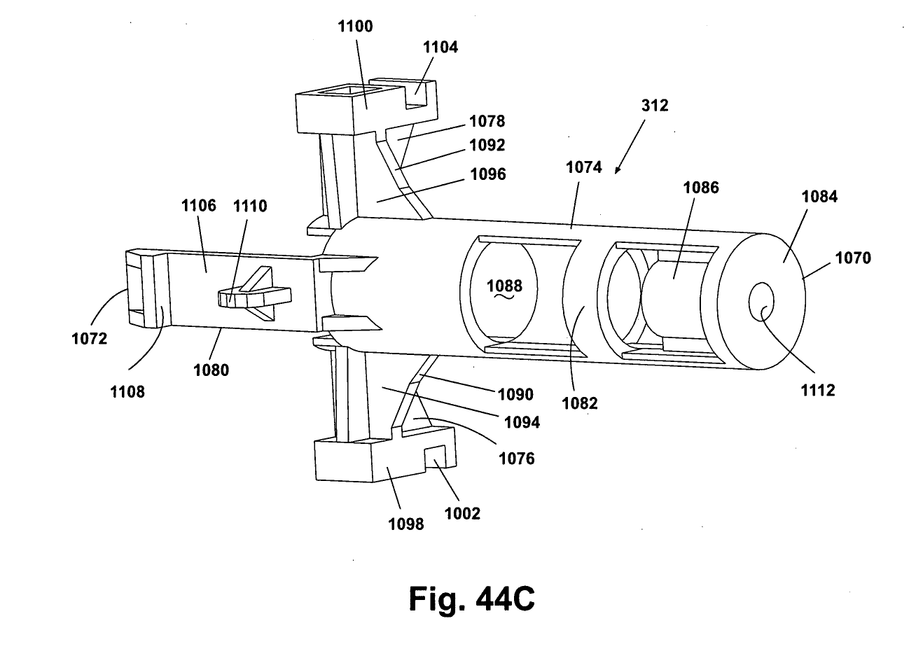

As illustrated in FIGS. 44A-C, the spoon cannula carriage 312 has a distal end 1070 and a proximal end 1072, and comprises a barrel portion 1074, a pair of wing portions 1076, 1078, and a hook portion 1080. The barrel portion 1074 is an elongated, cylindrically-shaped body comprising an annular wall 1082 terminating at the distal end 1070 and a circular end wall 1084. The annular wall 1082 and the end wall 1084 define a cylindrical barrel chamber 1088. Extending orthogonally from the end wall 1084 into the barrel chamber 1088 is a cylindrical spoon cannula seat 1086 having a circular aperture 1112 extending coaxially through the spoon cannula seat 1086 and the end wall 1084.

Each wing portion 1076, 1078 comprises a beam 1090, 1092, respectively, extending diametrically outwardly from a proximal end of the barrel portion 1074. Each beam 1090, 1092 is provided with a triangular brace 1094, 1096, respectively, extending distally of the beam 1090, 1092 to join the barrel portion 1074. Each brace 1094, 1096 terminates in a rectilinear end bearing 1098, 1100, respectively, having an elongated, rectilinear end slot 1102, 1104, respectively, at a lower portion thereof parallel to the longitudinal axis of the barrel portion 1074.

The hook portion 1080 comprises an elongated member extending longitudinally away from a proximal end of the barrel portion 1074, disposed 90° from each wing portion 1076, 1078 parallel to the longitudinal axis of the barrel portion 1074. The hook portion 1080 comprises a strap-like resilient arm 1106 attached to the barrel portion 1074 in cantilevered fashion, terminating in an upwardly-disposed hook 1108. Extending from the resilient arm 1106 adjacent the connection of the arm 1106 with the barrel portion 1074 is an upwardly-disposed, plate-like fin 1110 aligned with the longitudinal axis of the barrel portion 1074. The barrel portion 1074 is adapted for slidable registry with the cradle opening 835 of the shuttle 170, and the braces 1094, 1096 are adapted for slidable receipt in the notches 838 of the wings 832, 834.

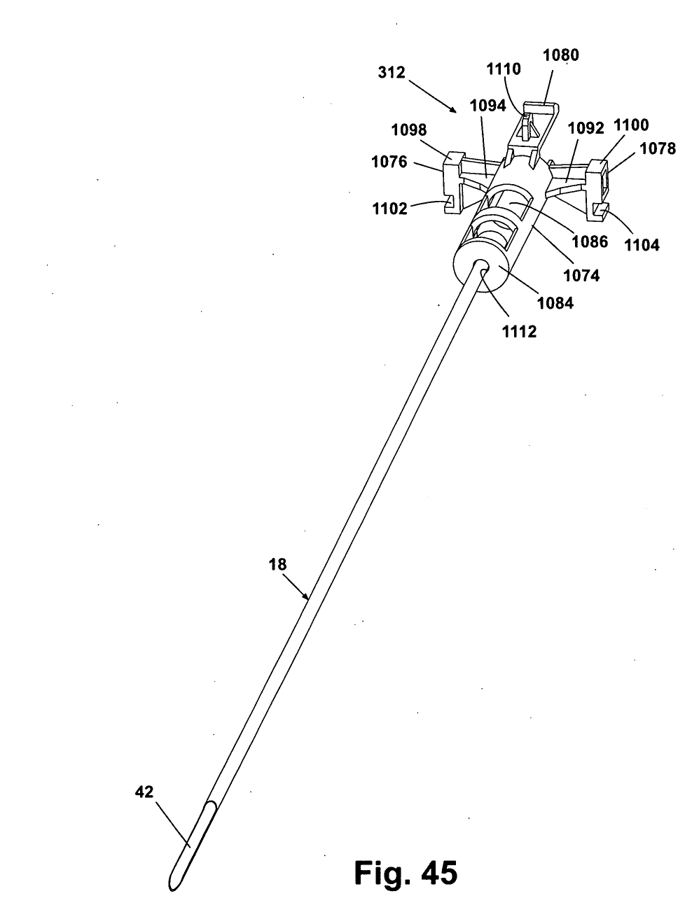

As illustrated in FIG. 45, a proximal end of the spoon cannula 18 is received in the aperture 1112 for fixed attachment to the spoon cannula seat 1086 to extend distally away from the end wall 1084. The spoon cannula 18 is oriented in the spoon cannula seat 1086 so that the spoon section 42 opens dorsally in longitudinal alignment with the fin 1110.

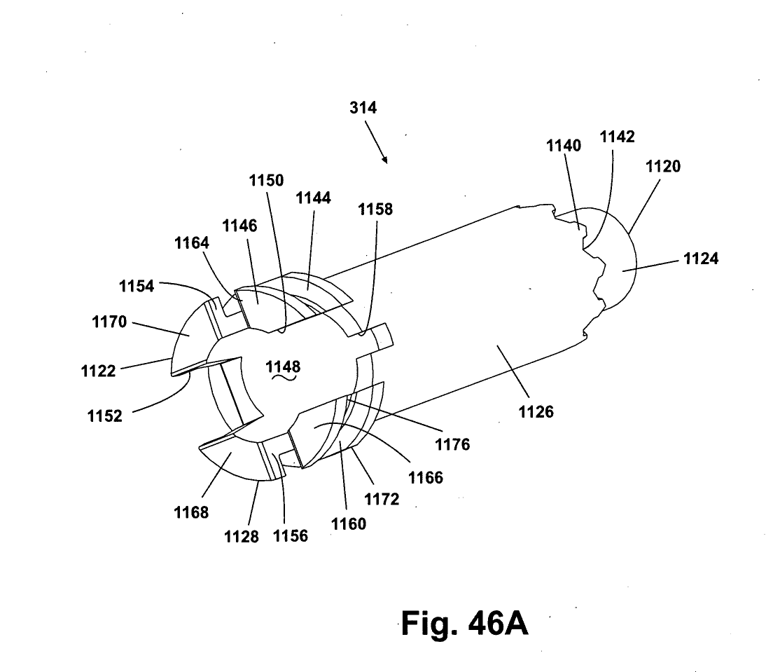

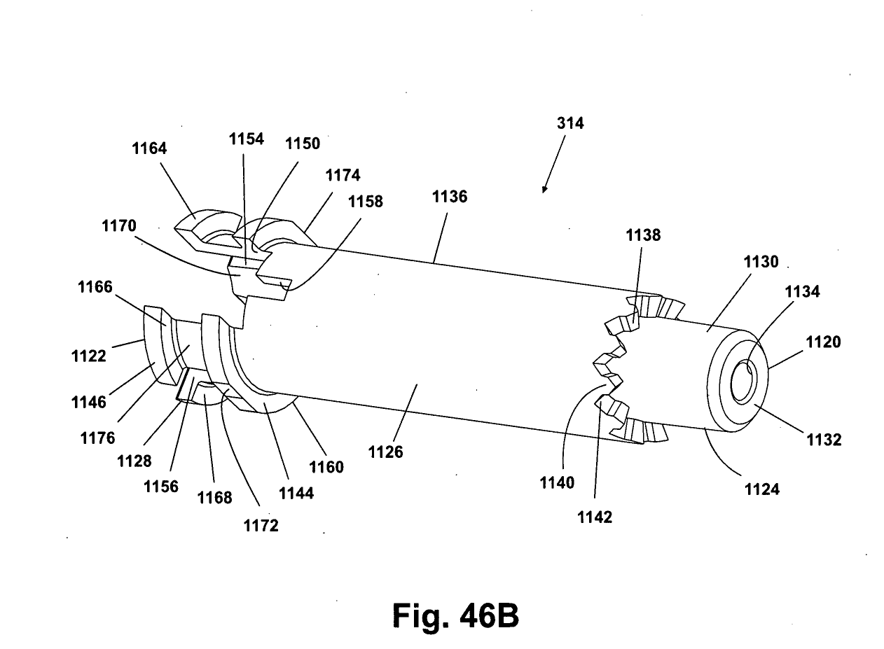

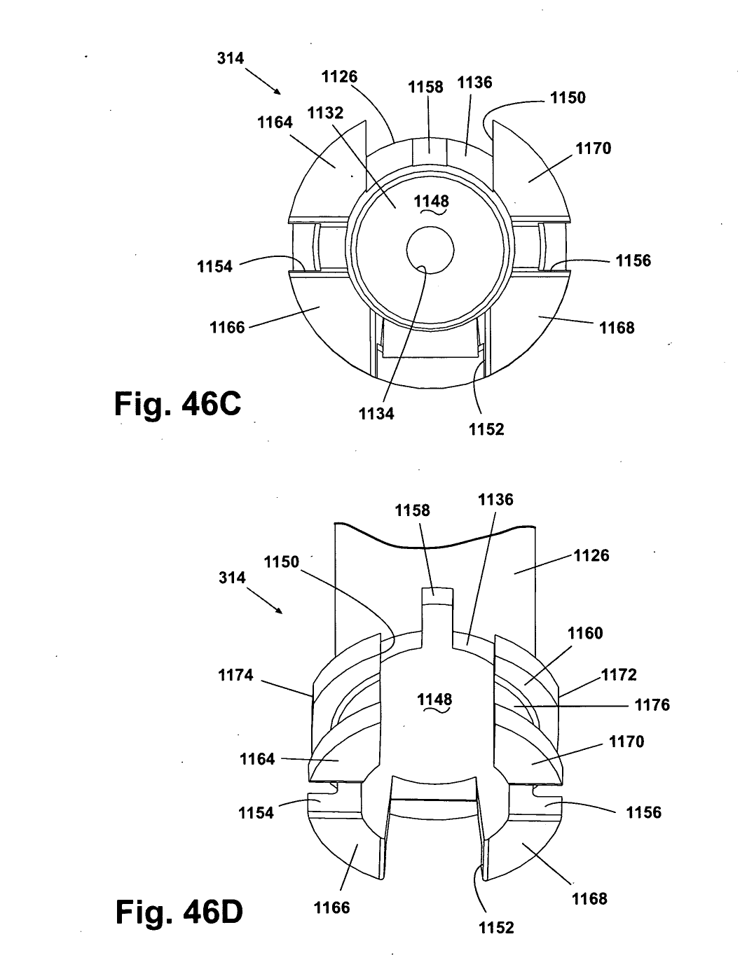

As illustrated in FIGS. 46A-D, the cutting cannula carriage 314 is an elongated, generally cylindrically-shaped body having a distal end 1120 and a proximal end 1122, and comprising a nose portion 1124 at the distal end 1120, a flange portion 1128 at the proximal end 1122, and a barrel portion 1126 intermediate the nose portion 1124 and the flange portion 1128. The nose portion 1124 comprises an annular wall 1130 terminating at the distal end in a circular end wall 1132 having an aperture 1134 coaxially extending therethrough. The barrel portion 1126 comprises an annular wall 1136 having a diameter somewhat greater than the diameter of the annular wall 1130.

The annular wall 1136 terminates at its intersection with the nose portion 1124 in a circumferential toothed edge 1138 comprising a radial array of alternating projections 1140 and cavities 1142, which effectively form a gear. The annular wall 1136 transitions at the proximal end 1122 to a circumferential inner flange 1144 extending radially away from the annular wall 1136. The inner flange 1144 transitions through a neck portion 1176 having a diameter equal to the diameter of the annular wall 1136 to a circumferential outer flange 1146 extending radially away from the neck portion 1176 in parallel, spaced disposition with the inner flange 1144.

The outer flange 1146, the annular wall 1136, and the inner flange 1144 are interrupted by a primary notch 1150 inset in the annular wall 1136 from the proximal end 1122. A narrow barrel notch 1158 is inset longitudinally from the primary notch 1150 into the annular wall 1136 toward the distal end 1120. The inner flange 1144 is provided with a pair of planar faces 1172, 1174 along the circumferential face thereof spaced 90° from the primary notch 1150. The outer flange 1146 is interrupted by a primary notch 1152 in diametrically-opposed disposition with the primary notch 1150, and a pair of secondary notches 1154, 1156 in diametrically-opposed disposition and offset 90° from the primary notches 1150, 1152. The notches 1150-1156 divide the outer flange 1146 into equal area sector pieces 1164, 1166, 1168, 1170. The outer flange 1146, the inner flange 1144, the neck portion 1176, the annular wall 1136, and the annular wall 1130 define a cylindrical chamber 1148 extending from the proximal end 1122 to the aperture 1134. The aperture 1134 is adapted for fixed insertion of the coring cannula 16 so that the coring cannula 16 extends distally from the distal end 1120. The coring cannula 16 is oriented relative to the cutting cannula carriage 314 so that the excising finger 70 is longitudinally aligned with the barrel notch 1158.

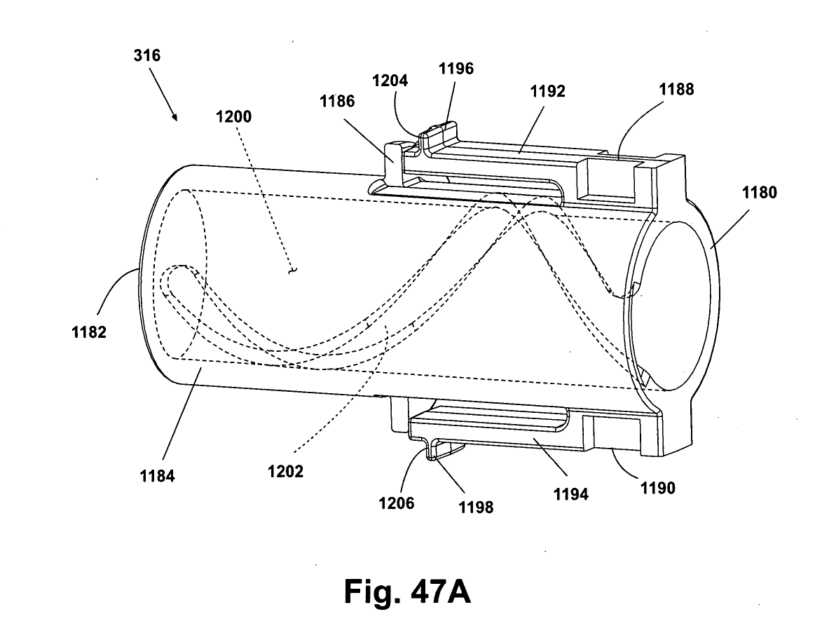

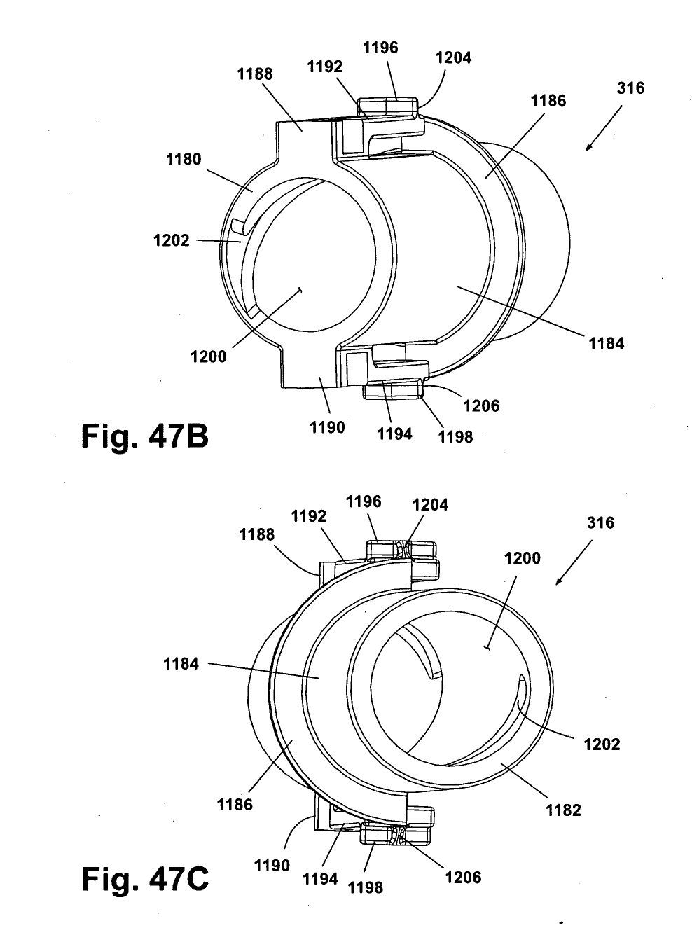

Referring now to FIGS. 47A-C, the helical drive member 316 is an elongated, generally annular body having a distal end 1180 and a proximal end 1182, and comprising an annular wall 1184 defining a cylindrical chamber 1200 open at both ends 1180, 1182. A helical channelway 1202 extends along the interior of the annular wall 1184 from the distal end 1180 to the proximal end 1182. A semicircular rib 1186 extends radially along an outer surface of the annular wall 1184 at approximately the mid-section of the annular wall 1184. A pair of diametrically-opposed resilient arms 1192, 1194 extends longitudinally along the outer surface of the annular wall 1184 from the distal end 1180 toward the proximal end 1182. The resilient arms 1192, 1194 are attached in cantilevered fashion to the annular wall 1184 through a pair of diametrically-opposed mounting blocks 1188, 1190 at the distal end 1180. The resilient arms 1192, 1194 terminate in a pair of radially outwardly-extending hooks 1196, 1198, respectively. An inclined brace 1204, 1206 extends from the resilient arm 1192, 1194, respectively, to the proximal face of the hook 1196, 1198, respectively.

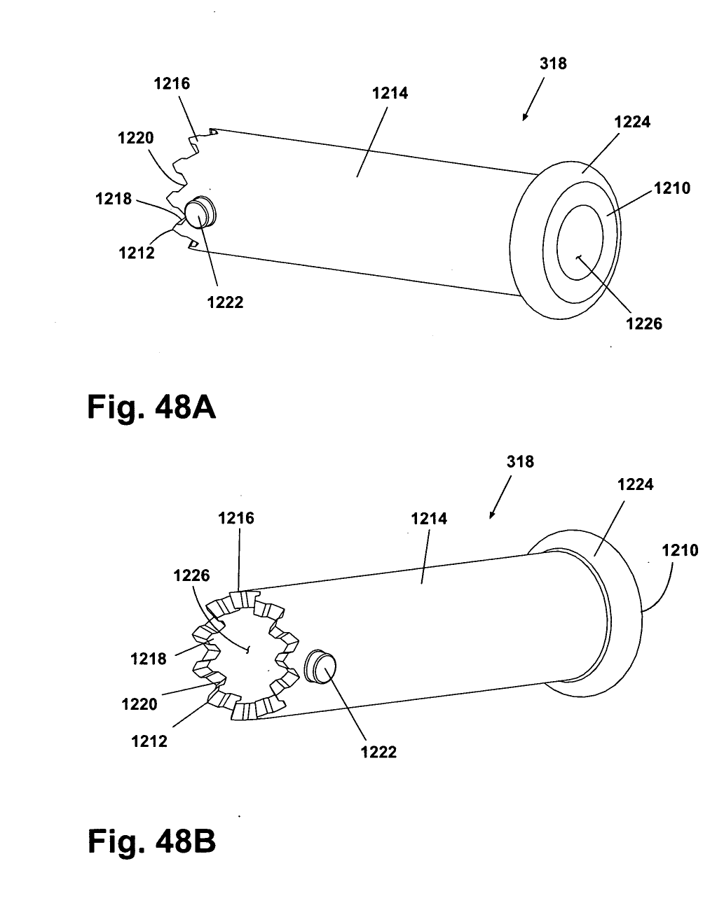

Referring now to FIGS. 48A-B, the rotating driven member 318 is an elongated, generally annular body having a distal end 1210 and a proximal end 1212, and comprising an annular wall 1214 defining a cylindrical aperture 1226 extending between both ends 1210, 1212. The annular wall 1214 terminates at the proximal end 1212 in a circumferential toothed edge 1216 comprising a radial array of alternating projections 1218 and cavities 1220 that effectively form a gear that can mesh with the gear on the cutting cannula carriage 314. The projections 1218 are adapted for cooperative registry with the cavities 1142 of the cutting cannula carriage 314, and the cavities 1220 are adapted for cooperative registry with the projections 1140 of the cutting cannula carriage 314.

The annular wall 1214 transitions at the distal end 1210 to a circular flange 1224 having a diameter somewhat greater than the diameter of the annular wall 1214. A cylindrical boss 1222 extends radially away from the annular wall 1214 adjacent the proximal end 1212, and is adapted for slidable registry with the helical channelway 1202. The annular wall 1214 is adapted for slidable insertion in the chamber 1200 of the helical drive member 316, with the boss 1222 received in the channelway 1202. The aperture 1226 at the proximal end 1212 is adapted to receive the nose portion 1124 of the cutting cannula carriage 314, and the aperture 1226 at the distal end 1210 is adapted to receive the collar 996 of the nosepiece 300.

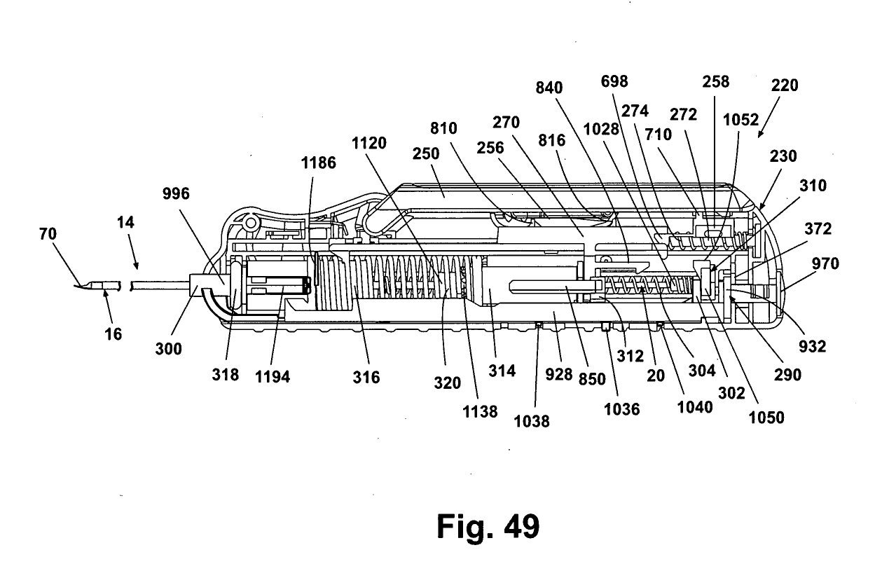

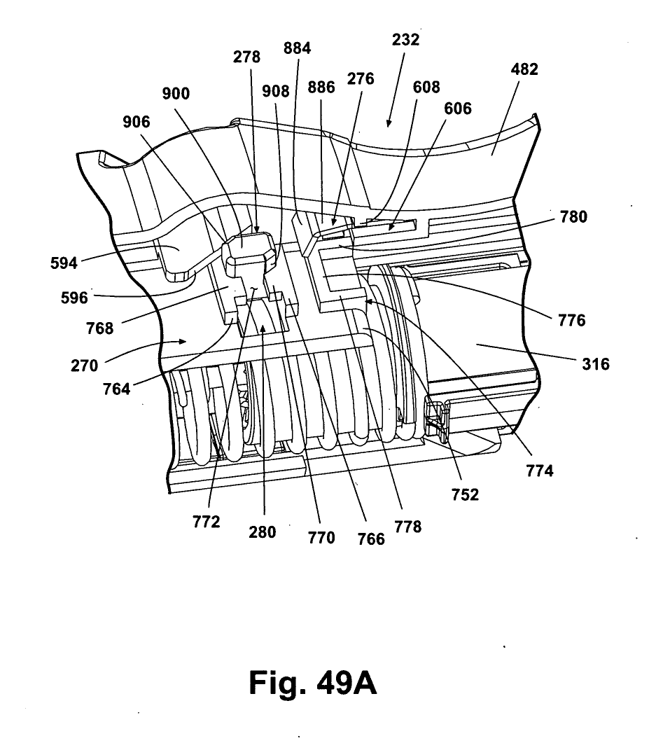

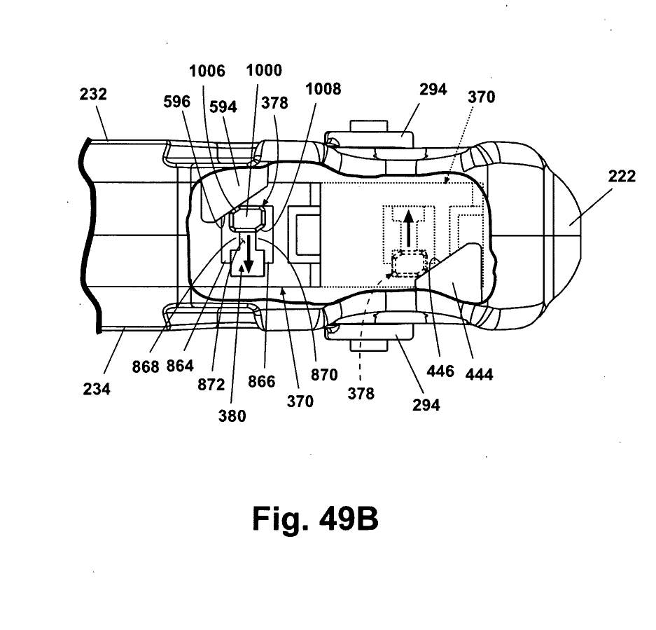

The assembled biopsy gun 220 will now be described with reference to the Figures, and particularly to FIGS. 25 and 49. The rotating driven member 318 is received in the helical drive member 316 by inserting the proximal end 1212 in the distal end of the chamber 1200 with the boss 1222 received in the helical channelway 1202. The cutting cannula carriage 314, with the coring cannula 16 affixed thereto, is installed in the helical drive member 316 by inserting the distal end 1120 in the proximal end of the chamber 1200 for cooperative registry of the toothed edge 1138 of the cutting cannula carriage 314 with the toothed edge 1216 of the rotating driven member 318. The cutting cannula carriage 314 and the coring cannula 16 are oriented so that the excising finger 70 is to the ventral side of the biopsy gun 220 and the barrel notch 1158 is to the ventral side of the cutting cannula carriage 314. The semicircular rib 1186 is oriented to the dorsal side of the helical drive member 316. A helical spring 320 encircles the proximal end 1182 of the helical drive member 316 to bear against the semicircular rib 1186 and extend away from the proximal end 1182.

The spoon cannula carriage 312, with the spool cannula 18 affixed thereto, is received in the cutting cannula carriage 314 by inserting the distal end 1070 into the proximal end 1122 of the chamber 1148 with the spoon cannula 18 inserted through the coring cannula 16. The helical spring 304 is inserted into the barrel chamber 1088 of the spoon cannula carriage 312 and retained by the spoon cannula seat 1086 to extend proximally away from the spoon cannula carriage 312. With the stylet 20 seated in the stylet seat 1028 of the adjustment member 302, the assembled spoon cannula carriage 312, cutting cannula carriage 314, helical drive member 316, and rotating driven member 318 are attached to the adjustment member 302 by inserting the stylet 20 through the spoon cannula 18. The proximal end of the helical spring 304 is retained around the stylet seat 1028, thereby urging the spoon cannula carriage 312 distally into engagement with the cutting cannula carriage 314. The adjustment member 302 is inserted into the slot 950 of the firing cage 290 so that the side rails 926, 928 and the beam 1024 of the adjustment member 302 are in longitudinal registry to form a curved, closed-bottom channel. The nosepiece 300 is then attached to the adjustment member 302 by inserting the cannula assembly 14 through the collar 996 and the aperture 1010, and inserting the dovetail 1002 into the dovetail cutout 1042 so that the collar 996 is received in the aperture 1226 of the rotating driven member 318, with the mortise 1000 of the nosepiece beam 994 in slidable registry with the end wall slot 952. As so assembled, the sample size control assembly 242 can translate longitudinally relative to the firing cage 290 a distance defined by the length of the mortise 1000.

The firing cage 290 is received in one of the housing shells 232, 234, such as the right housing shell 234, so that the plunger 970 is seated in the trigger opening 360 and the spring arm 972 contacts the intermediate wall 364. The beam 968 is received in the intermediate trigger opening 370 and the distal trigger opening 374. The side rail 926 and the beam 1024 extend along the bottom wall 334, with the collar 996 of the nosepiece 300 extending through the nosepiece opening 454 and the nosepiece opening 448. The adjustment lever 1036 is received in the sample size selector slot 410, the forward stop 1038 is received in one of the distal slots 412, 414, and the rearward stop 1040 is received in one of the proximal slots 416, 418. The distal end wall 930 of the firing cage 290 will be positioned adjacent the distal end of the front trigger opening 460 so that the tooth 466 will be in cooperative registry with the inclined face 946. This enables the button 294 attached to the button boss 468 to be inwardly-depressed so that the tooth 466 can be urged inwardly against the inclined face 446, thereby urging the firing cage 290 in a distal direction. The resilient arm 1192 and the mounting block 1188 of the helical drive member 316 are received within the slot 435 in the side wall 330 of the right housing shell 234 distally of the stop rib 433 for slidable translation therein.

The latch plate 310 is supported by the latch plate support 390 with the plate portion 1050 received in the latch plate channel 392 so that the crown portion 1052 extends distally with the proximal end wall 932 of the firing cage 290 positioned intermediate the latch plate 310 and the inner wall 372. The spoon cannula carriage 312 is supported by the upper spoon cannula carriage rail 394 and the lower spoon cannula carriage rail 396, with the end slot 1102 of the end bearing 1098 in slidable registry with the lower spoon cannula carriage rail 396, and the end bearing 1098 slidably received between the carriage rails 394, 396. The cutting cannula carriage 314 is supported by the cutting cannula carriage cradle 430 along the barrel portion 1126. The helical drive member 316 is also supported by the arcuate surface 942 of the distal end wall 930 of the firing cage 290.

The latch 258 is installed by inserting the lateral beam 716 outwardly through the latch slot 350 so that a button 260 can be affixed to the button mount 724. The flange plate 720 will be in slidable registry with the dorsal face of the upper interior wall 380, with the tooth 702 in registry with the dorsal side of the upper interior wall 530 proximal of the lower latch opening 384. The hook 712 will extend vertically upwardly through the upper latch opening 346. The box portion 756 of the shuttle 270 will extend through the shuttle cavity 726. An elastic band 728 of suitable resiliency and dimension is extendable around each latch arm 708, 710 and the retainer boss 526 to urge the latch 258 toward the proximal wall 486. The latch 258 can be selectively translated alternately proximally and distally by movement of the button 260 and the lateral beam 716 along the latch slot 350.

The shuttle 270 is received in the right housing shell 234 with the distal end 752 of the shuttle 270 extending toward the distal wall 488 of the right housing shell 234 so that the lateral edge of the plate portion 754 and the end boss 828 are received in the shuttle slot 386. The lower rail 852 of the shuttle 270 is received in the slot 435 proximally of the stop rib 433. The side wall 802 of the box portion 756 will be in slidable registry with the swing arm cage 339. The box portion 756 will extend dorsally of the upper interior wall 380. The cam block retainer 280 and the end box 774 will extend dorsally approximately the same height as the upper interior wall 380. The arcuate surface 790 of the cradle wall 786 will slidably register with the annular wall 1184 of the helical drive member 316 with the cradle wall 786 in registry with the distal side of the semicircular rib 1186. The cam block 278 is slidably inserted into the cam block retainer 280 so that the inner flange 768 and the outer flange 770 of the cam block retainer 280 are received in the channelways 910, 912 and the upper block 900 extends dorsally of the flanges 768, 770. The spring retainer 272 is slidably inserted into the chamber 820 of the box portion 756 so that the spring 274 encircles the rod 870 of the spring retainer 272 and the spring boss 864 of the box portion 756. The flange 872 of the spring retainer 272 can then be inserted into the spring retainer chamber 366 of the right housing shell 234 with the rod 870 extending through the spring retainer opening 368.

The left housing shell 232 can be assembled to the right housing shell 234 with the internal components seated in and supported by the cooperative elements of both the left housing shell 232 and the right housing shell 234.

With the left housing shell 232 and the right housing shell 234 assembled into the housing 230, the handle 250 can be installed to the housing 230 by inserting the pivot bosses 636 into the pivot apertures 442, 592. The spring mounting bosses 646 are inserted into the center apertures 660 of the mounting arm 650 for frictional registry of the mounting arm 650 with the handle 250. The mounting arm 650 is oriented to extend intermediate the swing arm supports 642 with the contact arm 652 depending away from the handle 250 proximally of the swing arm supports 642. The pivot rod 680 of the swing arm 256 is snapfit into the pivot openings 644 of the swing arm supports 642 so that the sliding rod 676 is disposed away from the handle 250 and the swing arm 256 is disposed distally of the contact arm 652. The end boss 678 is received in the swing arm chamber 342 for slidable translation therealong between the swing arm opening 340 and the proximal end 344. The end boss 678 can be inserted into the swing arm chamber 342 through the swing arm opening 340, and will be housed in the proximal end 344 with the swing arm support 642 received through the swing arm opening 340 when the handle 250 is pivoted ventrally to a closed position in registry with the housing 230.

As illustrated in FIG. 49A, the cam spring 276 is attached to the left housing shell 232 so that the upper leg 880 is received in the cam spring housing 606 with the inclined member 890 in registry with the stop wall 608 and the lower leg 884 depending toward the distal end 752 of the shuttle 270. The opposite shell, in this example the right housing shell 234, is then brought into mating registry with the left housing shell 232 to form the housing 230. The assembled elements will cooperatively engage the right housing shell 234 in a manner similar to the engagement of the assembled elements with the left housing shell 232, as hereinbefore described.

The operation of the biopsy gun 220 will now be described with reference to FIGS. 49-56. In the Figures, elements of the biopsy gun 220, particularly the housing shells 232, 234, will be either removed or illustrated in phantom to facilitate a complete understanding of the operation of the gun 220. The biopsy gun 220 is initially in an uncocked condition, and must be cocked prior to introducing the cannula assembly 14 into the tissue mass 22. As described previously herein and illustrated in FIG. 9A, with the biopsy gun 220 in a cocked condition, the cannula assembly 14 is inserted into the tissue mass 22 so that the penetration tip 34 is adjacent the lesion 24. The biopsy gun 220 is then fired by operation of the firing cage 290 for excision of a biopsy sample.

As illustrated in FIG. 49, in the uncocked condition, the biopsy gun 220 is initially in a configuration with the handle 250 extending along the dorsal side 226 of the housing 230. The latch 258, under the influence of the elastic band 728, is urged towards the proximal end 224 of the housing 230 with the hooks 710, 712 of the latch 258 in cooperative registry with the hook 640 of the latch extension 638 to retain the handle 250 against the housing 230. Under the influence of the spring 274, the shuttle 270 is urged toward the distal end 222 of the housing 230. Under the influence of the spring 304, the spool cannula carriage 312 and the cutting cannula carriage 314 are also urged toward the distal end 222 of the housing 230. The spring 254 is deflected into a generally linear configuration, with the swing arm 256 partially received in the housing 230. The cam block 278 will be urged to the left side of the housing 230 by engagement of the inclined face 906 with the inclined face 446 of the distal cam block wedge 444 extending inwardly from the right housing shell 234. The cam block 278 will thus be longitudinally aligned with the cam spring 276.

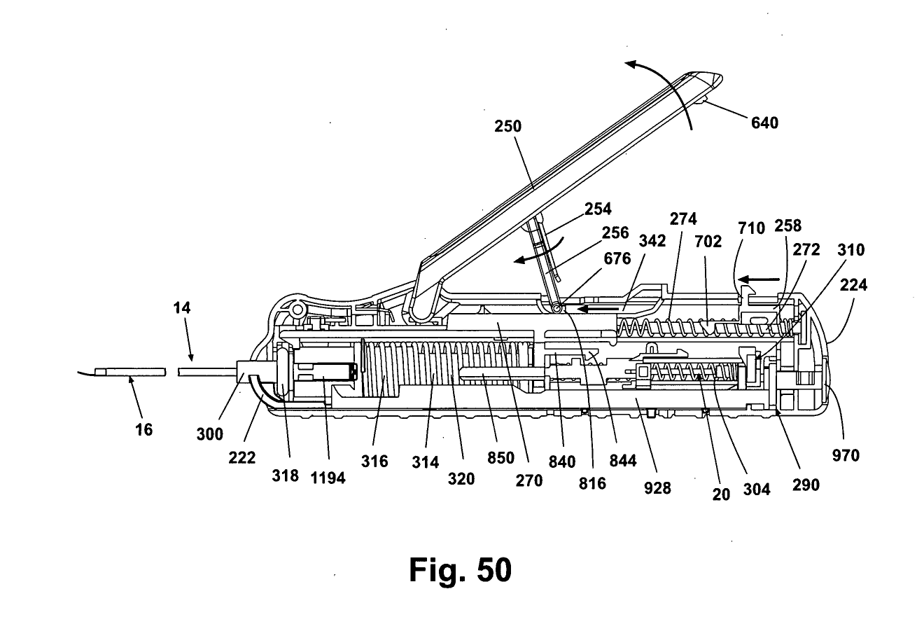

As illustrated in FIG. 50, the handle 250 is released by translating the buttons 260 at the proximal end 224 toward the distal end 222, which will translate the hooks 710, 712 away from the hook 640, and will “drop” the teeth 702, 704 into an interference registry with the lower latch opening 534, 384, respectively. This will prevent the translation of the latch 258 toward the proximal end 224, notwithstanding the tension exerted by the elastic band 728 tending to urge the latch 258 toward the proximal end 224. The handle 250 will be urged into an open position by the spring 254, which will also move the swing arm 256 to an oblique orientation as the end bosses 678 translate from the proximal end 344, 494 along the swing arm chamber 342, 492 to the swing arm opening 340, 490. The sliding rod 676 translates along the swing arm chamber 342, 492 and the top wall 800 of the shuttle 270, and over the outer bearing wall 816 into cooperative registry with the arcuate surface 818 of the outer bearing wall 816 extending dorsally of the top wall 800 of the shuttle 270.

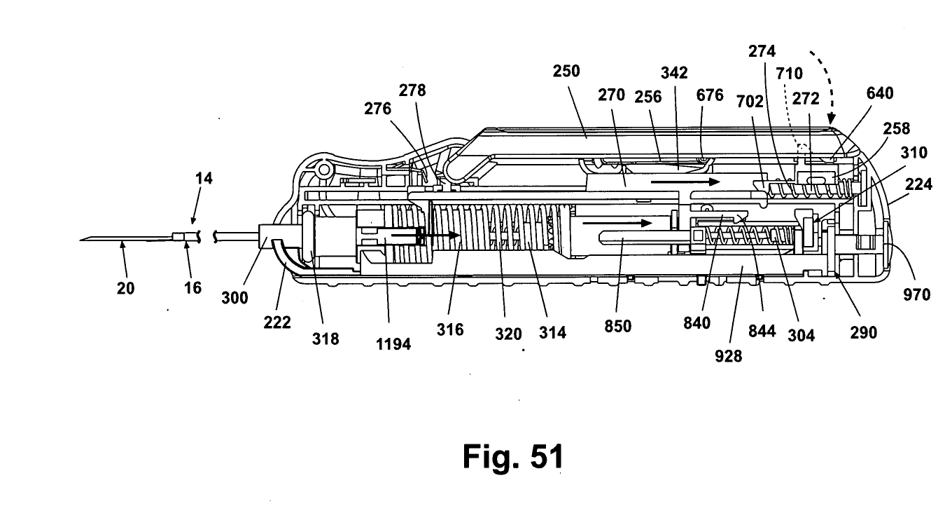

Referring now to FIG. 51, with the sliding rod bearing against the outer bearing wall 816, depressing the handle 250 toward the shell 230 will translate the shuttle 270 toward the proximal end 224. This will urge the helical drive member 316 toward the proximal end 224 due to the registry of the cradle wall 786 with the semicircular rib 1186, thereby compressing the spring 320 between the semicircular rib 1186 and the cutting cannula carriage cradles 430, 580. The resilient arms 1192, 1194 of the helical drive member 316 will be deflected radially inwardly by engagement of the braces 1204, 1206, respectively, with the stop ribs 583, 433 of the left and right housing shells 232, 234, respectively, until the hooks 1196, 1198 clear the ribs 583, 433 to return to an undeflected position in which the hooks 1196, 1198 are in registry with the proximal face of the ribs 583, 433, respectively, thus preventing the helical drive member 316 from moving toward the distal end 222 under the influence of the spring 320. The coring cannula 16 will be drawn over the stylet 20 and the spoon cannula 18 in a proximal direction.

During movement of the shuttle 270 toward the proximal end 224, the cam block 278 will be brought beneath the cam spring 276 thereby causing deflection of the lower leg 884 toward the dorsal side 226 of the housing 230. When the cam block 278 moves past the cam spring 276, the lower leg 884 will return to its initial at-rest position. The handle 250 can be fully depressed toward the shell 230 without engaging the latch 258 due to the retention of the latch 258 toward the distal end 222 and the registry of the hooks 702, 704 with the lower latch openings 384, 534, as previously described.

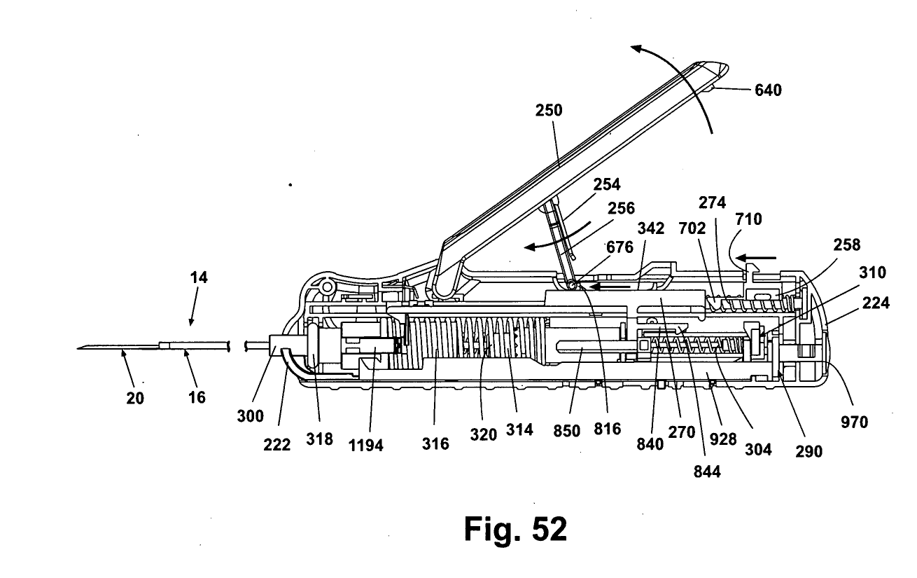

As illustrated in FIG. 49A, the shuttle 270 will be prevented from returning to its initial position by the interference between the cam block 278 and the lower leg 884 of the cam spring 276, which will engage the upper block 900 and prevent movement of the shuttle 270 toward the distal end 222. As illustrated in FIG. 52, the handle 250 can again be released to the open position by the operator merely opening his or her hand, without the necessity of translating the buttons 260 toward the distal end 222 as before, with the sliding rod 676 brought into cooperative registry with the arcuate surface 812 of the inner bearing wall 810 extending dorsally of the top wall 800 of the shuttle 270, since the inner bearing wall 810 will now be positioned where the outer bearing wall 816 was previously positioned. The inclined brace 814 will facilitate movement of the sliding rod 676 from the outer bearing wall 816 into engagement with the arcuate surface 812.

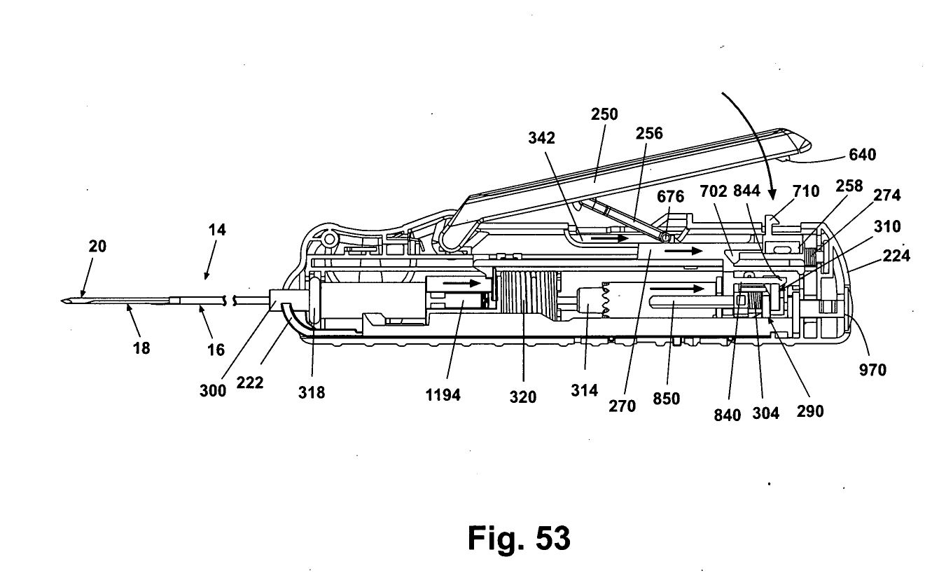

As illustrated in FIG. 53, depressing the handle 250 toward the shell 230 will again translate the shuttle 270 toward the proximal end 224. As the shuttle 270 translates toward the proximal end 224, the end bosses 826, 828 extending laterally away from the box portion 756 will engage the teeth 702, 704, respectively, of the latch 258, urging the teeth 702, 704 upwardly out of the lower latch openings 534, 384, respectively. Under the influence of the elastic band 728, the latch 258 will be urged toward the proximal end 224 for engagement with the handle 250. Thus, the handle 250 will be retained in a closed position against the housing 230 without the necessity of resetting the position of the latch 258. This structure permits the user to release the handle, squeeze the handle twice to cock the actuator, with the handle automatically springing up after the first squeezing of the handle, and locking in the closed position upon the second squeezing, which enable one-handed cocking of the actuator.

The hooks 844, 846 extending from the intermediate wall 830 of the shuttle 270 will engage the plate portion 1050 of the latch plate 310 on both sides of the crown portion 1052, thus retaining the shuttle 270 at its proximal limit, with the spring 274 fully compressed. The spool cannula carriage 312 and the cutting cannula carriage 314 will also be translated proximally by the shuttle 270 as a result of the cradle 835 being received in the neck portion 1176 of the cutting cannula carriage 314. This will bring the hook 1108 extending from the spool cannula carriage 312 into an interference engagement with the opening 1060 in the crown portion 1052 of the latch plate 310. The spring 304 will also be fully compressed. The brace 1030 of the adjustment member 302 will be received in the barrel notch 1158 of the cutting cannula carriage 314. The braces 1094, 1096 of the spoon cannula carriage 312 will be received in the secondary notches 1154, 1156 of the cutting cannula carriage 314, and the notches 838 in the intermediate wall 830 of the shuttle 270, thereby ensuring nesting of the spoon cannula carriage 312, the cutting cannula carriage 314, and the shuttle 270.

At the same time, the helical drive member 316 will be translated toward the distal wall 222 by engagement of the cradle wall 786 of the shuttle 270 with the semicircular rib 1186. This will fully compress the spring 320 between the semicircular rib 1186 and the cutting cannula carriage cradle 430, 580. Finally, as illustrated in FIG. 49B, the cam block 278 will be urged to the right side of the housing 230 by engagement of the inclined face 906 with the inclined face 596 of the proximal cam block wedge 594 extending inwardly from the left housing shell 232. In this configuration, the coring cannula 16 and the spoon cannula 18 will be fully retracted proximally, and the stylet 20 will extend distally of the coring cannula 16 and the spoon cannula 18, with the biopsy gun 220 in a cocked and ready position for obtaining a biopsy sample. The cannula assembly 14 can be introduced into the tissue mass 22 and advanced to the lesion 24 for recovery of the biopsy sample 26.

When the cannula assembly 14 has been positioned for recovery of the biopsy sample 26, the biopsy gun 220 is actuated. The biopsy gun 220 can be actuated in one of two ways to obtain the biopsy sample 26. Both procedures actuate the firing cage 290. In the first procedure, the firing plunger 292 is depressed toward the distal end 222. In the second procedure, the buttons 260 at the lower distal end of the housing 230 are depressed laterally inwardly. This urges the release arms 462, 612 laterally inwardly, which urges the teeth 466, 616 inwardly against the inclined faces 944, 946 of the distal end wall 930, thereby urging the firing cage 290 toward the distal end 222.

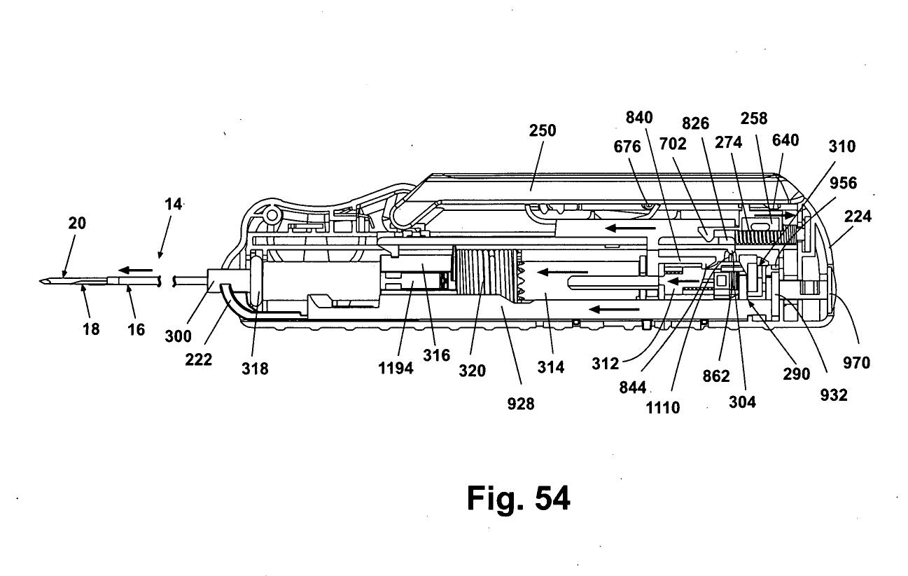

As illustrated in FIG. 54, as the firing cage 290 translates toward the distal end 222, the flanges 956, 958 of the proximal end wall 932 engage the hooks 844, 846 of the shuttle 270, urging the hooks 844, 846 dorsally out of engagement with the latch plate 310. This will release the shuttle 270 for translation toward the distal end 222 under the influence of the spring 274. This will translate the cutting cannula carriage 314 toward the distal end 222, which will initiate the movement of the coring cannula 16 over the spoon cannula 18, with the excising finger 70 translating along the arcuate wall 46 of the spoon cannula 18.

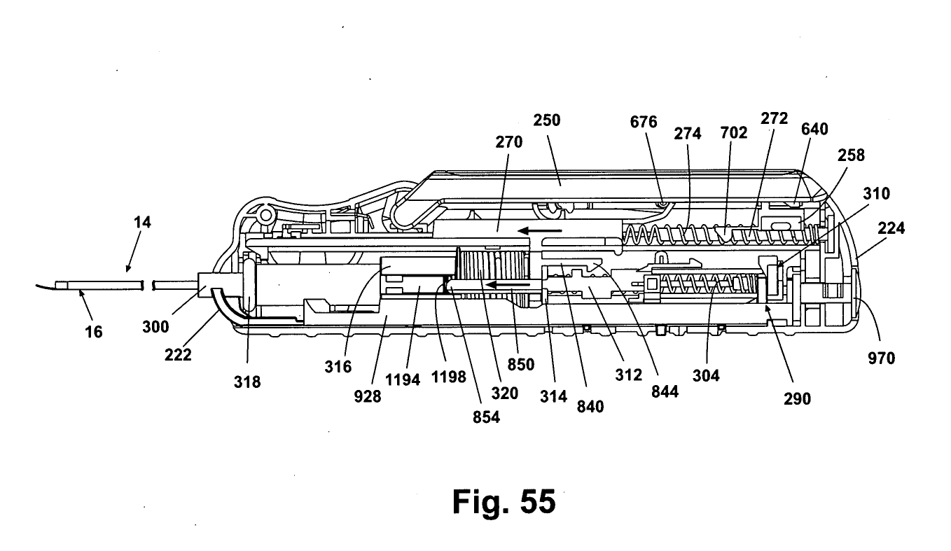

As the shuttle 270 continues to translate toward the distal end 222, the end boss 862 depending from the box portion 756 will engage the fin 1110 extending dorsally from the resilient arm 1106 of the spoon cannula carriage 312. This will deflect the resilient arm 1106 ventrally, releasing the hook 1108 from the latch plate 310, and enabling the spoon cannula carriage 312 to move toward the distal end 222 under the influence of the spring 304. The cutting cannula carriage 314 and the spoon cannula carriage 312 will thus translate toward the distal end 222, thereby urging the coring cannula 16 and the spoon cannula 18 into the lesion 24. The cutting cannula carriage 314 will be brought into engagement with the rotating driven member 318 and thus prevented from further translation toward the distal end 222. The spoon cannula carriage 312 will bottom out as the end bearings 1098, 1100 engage the stops 398, 400 in the right housing shell 234 and the stops 548, 550 in the left housing shell 232, respectively. The cutting cannula carriage 314 will bottom out after the spoon cannula carriage 312 bottoms out due to the engagement of the cutting cannula carriage 314 with the rotating driven member 318, so that the excising finger 70 extends distally of the insertion tip 48 of the spoon cannula 18, as illustrated in FIG. 55.

As the shuttle 270 continues to translate toward the distal end 222, the lower rails 850, 852 will translate in the slots 435, 585, toward the stop ribs 433, 583. The end portions 854, 858, of the lower rails 850, 852, respectively, will engage the resilient arms 1194, 1192, respectively. As illustrated in FIG. 55, the tapered tips 856, 860 of the end portions 854, 858, respectively, will engage the braces 1206, 1204, respectively, of the hooks 1198, 1196, urging the radially-inward deflection of the resilient arms 1194, 1192, respectively. This will urge the hooks 1198, 1196 out of registry with the stop ribs 433, 583, freeing the helical drive member 316 to translate toward the distal end 222 under the influence of the spring 320. As illustrated in FIG. 49B, the cam block 378 will be urged against the inclined face 446 of the distal cam block wedge 444, and translated along the gap 772 of the cam block retainer 280 toward the left housing shell 232.

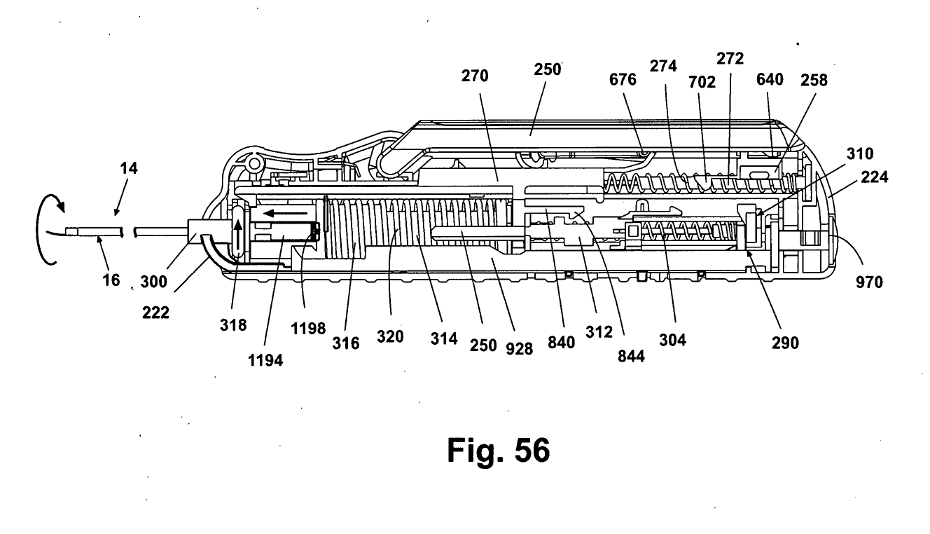

Referring to FIG. 56, as the helical drive member 316 translates, the rotating driven member 318 will be urged into rotation by the rotational motion of the helical channelway 1202 acting on the boss 1222. Rotation of the rotating driven member 318 will urge the rotation of the cutting cannula carriage 314 due to the interlocking of the teeth 1218 and channels 1220 of the rotating driven member 318 with the channels 1142 and teeth 1140, respectively, of the cutting cannula carriage 314. This will urge the excising finger 70 in a rotational motion, excising the sample 26 from the lesion 24. The cannula assembly 14 can then be withdrawn from the tissue mass 22 with the biopsy sample 26 retained in the spoon section 42 of the spoon cannula 18.

It is highly preferred that the rotation of the excising finger not begin until the excising finger has reached its longitudinal extent. This prevents the rotation of the excising finger from cutting a helical path in the sample. The helical channelway 1202, the helical drive member 316, and the boss 1222 are preferably adapted so that the cutting cannula carriage 314 and the coring cannula 16 rotate 1½ turns with a complete stroke of the helical drive member 316. More than one complete revolution increases the likelihood that the sample is completely severed.

While it is preferred to have more than one revolution of rotation for the excising finger to ensure separation, the amount of rotation needed to ensure the separation of the sample from the surround tissue can vary. In some cases, less than a complete revolution is sufficient. For example, when the excising finger extends substantially across the diameter of the coring cannula or when the suction and friction forces between the specimen and cannula are sufficiently high. Multiple revolutions can also be used. There is no theoretical limit to the number of revolutions. However, it is anticipated that 1½ revolution is sufficient.

As a result of the above-described firing of the biopsy gun 220, the coring cannula 16 is propelled over the spoon section 42 so that the excising finger 70 is positioned slightly proximally of the insertion tip 48 (FIG. 8B). The coring cannula 16 and the spoon cannula 18 then translate simultaneously into the lesion 24 to form the biopsy sample (FIG. 9B). The coring cannula 16 is further projected relative to the spoon cannula 18 so that the excising finger 70 disengages from the spoon cannula 18 to penetrate the tissue mass 22 distally of the biopsy sample (FIGS. 8C and 9C). The translation of the helical drive member 316 urges the boss 1222 along the helical channelway 1202, thereby urging the cutting cannula carriage 314 to rotate in a counterclockwise direction, thereby rotating the coring cannula 16 relative to the spoon cannula 18, so that the excising finger 70 excises the biopsy sample from the tissue mass 22 (FIG. 9D). The core biopsy device 10 is then removed from the tissue mass 22 with the biopsy sample enclosed therein (FIGS. 9E and 9F). Re-cocking of the biopsy gun 220 retracts the coring cannula 16 away from the spoon section 42, thereby exposing the biopsy sample 26 for removal (FIG. 9G).

Alternately, the biopsy gun 220 can be adapted to position the cannulae 16, 18 upon cocking of the biopsy gun 220 as shown in FIG. 8D. In this configuration, the enclosed section 60 of the coring cannula 16 is essentially coextensive with the enclosed section 40 of the spoon cannula 18, thus eliminating the semi-annular gap 88 between the stylet 20 and the annular wall 62 of the coring cannula 16. The distal edge of the excising finger 70 is in resilient contact with the arcuate wall 46 at the proximal end of the spoon section 42. The cannula assembly 14 is advanced into the tissue 22 as previously described, followed by the simultaneous advancement of the cannulae 16, 18 into the lesion 26. The coring cannula 16 is then advanced over the spoon cannula 18 through the lesion 26 until the excising finger 70 extends distally of the insertion tip 48 to position the coring cannula 16 for excision of the sample 26.

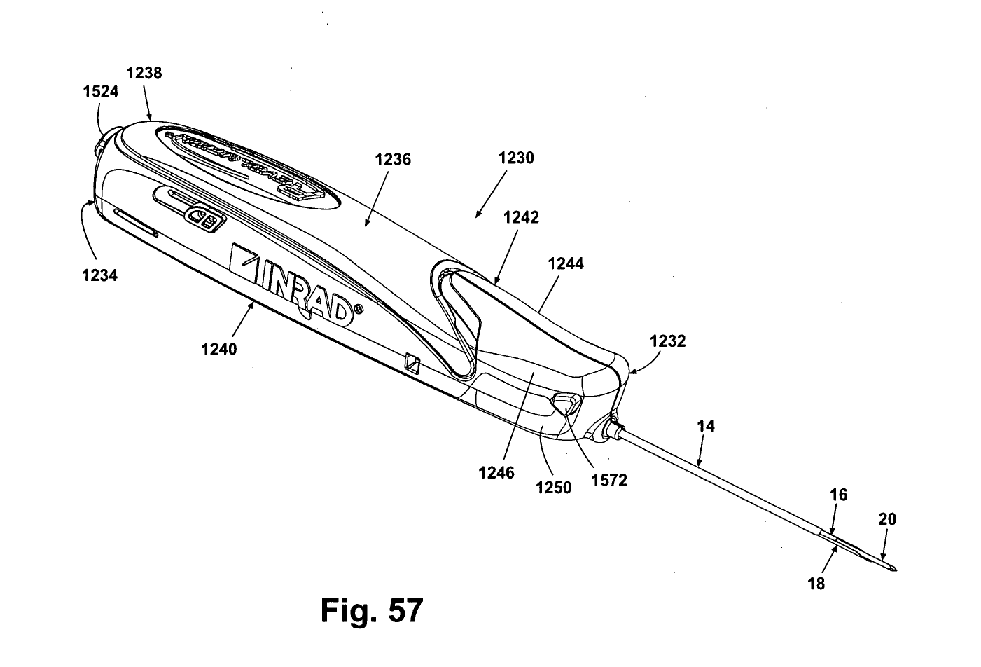

FIGS. 57-86 illustrate a third embodiment of an actuator assembly or biopsy gun 1230 for both translating the cannula assembly 14 to the excising position and then rotating the coring cannula 16. FIG. 57 illustrates the biopsy gun 1230 operably connected to the cannula assembly 14. The actuator assembly 12 has a distal end 1232, a proximal end 1234, a dorsal side 1236 supporting a cocking handle assembly 1238, and a ventral side 1240.

The biopsy gun 1230 comprises an outer housing 1242 providing an ergonomic, functional handle for facilitating the insertion of the cannula assembly 14 in a lesion 24 and the recovery of a biopsy sample 26, and comprising a left housing shell 1244 and a right housing shell 1246 adapted for cooperative registry, each housing shell 1244, 1246 having a corresponding overmolded portion 1248, 1250 for providing a soft gripping surface on the housing 1242. The housing shells 1244, 1246 are provided with a suitable number of connecting elements, such as snap-fit retainers, post and seat structures, threaded fastener apertures and seats, and the like, for fixedly interconnecting the housing shells 1244, 1246 to form the housing 1242.

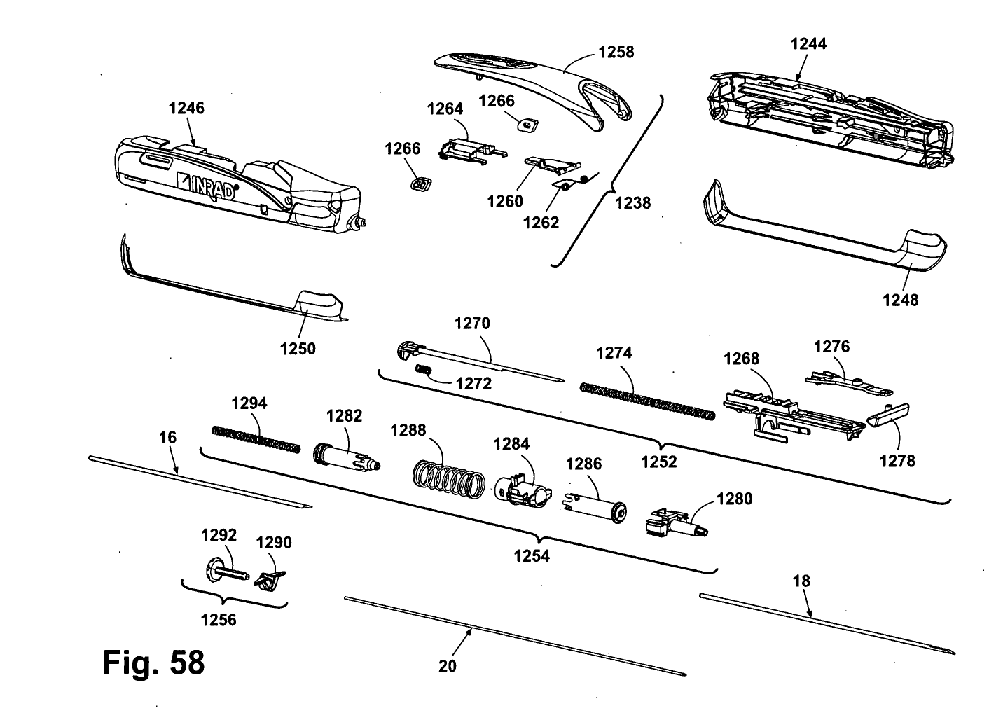

Referring to FIG. 58, the various components and/or assemblies of the biopsy gun 1230 are illustrated. The biopsy gun 1230 comprises the cocking handle assembly 1238, a trigger assembly 1252, a cannula operation assembly 1254 and a sample size control assembly 1256. The cocking handle assembly 1238 comprises a cocking lever 1258, swing arm 1260, a spring 1262, a latch 1264, and a pair of latch buttons 1266. The trigger assembly 1252 comprises a shuttle 1268, a trigger plunger 1270, a first spring 1272, a second spring 1274, a toggle piece 1276 and a toggle button 1278. The cannula operation assembly 1254 comprises a spoon cannula carriage 1280, a coring cannula carriage 1282, a helical drive member 1284, a rotating driven member 1286, a first spring 1288 and a second spring 1294. The sample size control assembly 1256 comprises a stylet carriage 1290, and an adjustment member 1292. These elements are interconnected and supported within the housing 1242 in and on various seats, slots, and rails facilitating the precisely controlled movement of the elements during the sample recovery process.

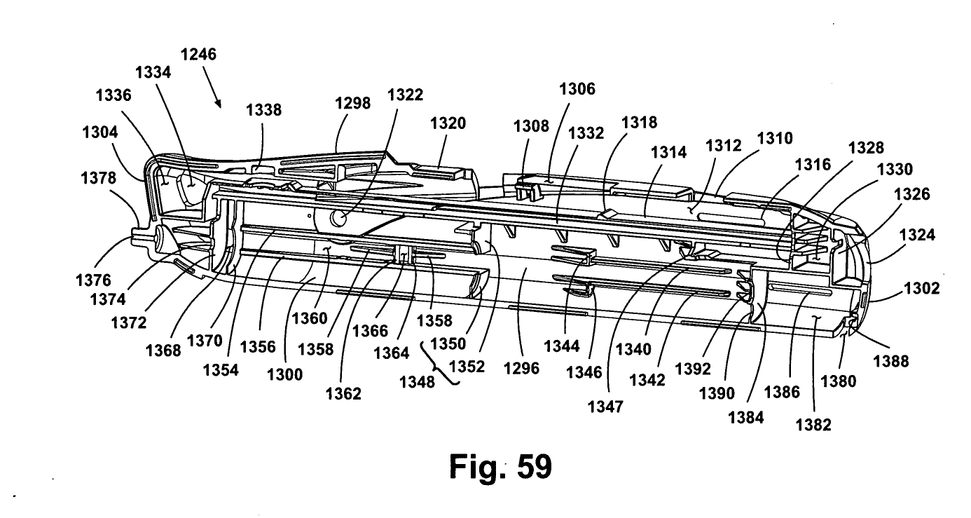

FIG. 59 illustrates the right housing shell 1246, which is an irregularly-shaped, elongated body comprising an elongated side wall 1296 joined to a top wall 1298, a bottom wall 1300, a proximal wall 1302, and a distal wall 1304. The walls 1296-1304 are contoured, and configured with openings, bosses, rails, and the like, for operational support of the elements comprising the biopsy gun 1230. The left housing shell 1244 is generally a mirror image of the right housing shell 1246, and has many of the same structural elements of the right housing shell 1246 arranged for cooperative registry of the structural elements in both shells 1244, 1246 to provide support and movement functionality to the assembled housing 1242. Accordingly, the term “chamber” as used in the description of the right handle shell 1246, unless otherwise noted, is used with the understanding that any such chamber is formed between structural elements of the assembled housing shells 1244, 1246.

Regarding operational support of the elements comprising the cocking handle assembly 1238, the top wall 1298 is provided with a swing arm opening 1306 having a swing arm stop 1308 protruding from one side of the swing arm opening 1306. Proximal of the swing arm opening 1306, the top wall 1298 is provided with a latch opening 1310 therethrough entering into a latch chamber 1312 at least partially defined by a laterally extending latch retaining wall 1314. A latch slot 1316 communicates with the latch chamber 1312 through the side wall 1296. A latch protrusion 1318 is formed on the latch retaining wall 1314 for cooperative registry with the latch 1264. The side wall 1296 and the top wall 1298 are formed with a depressed area 1320 conforming to the general shape of the cocking lever 1258, and the side wall 1296 is provided with a circular pivot aperture 1322 extending therethrough within the depressed area 1320.

Regarding operational support of the elements comprising the trigger assembly 1252, the right housing shell 1246 comprises a semicircular rear trigger opening 1324 in the proximal wall 1302. A flange receiver wall 1326 is positioned interiorly of the rear trigger opening 1324 and a spring retaining wall 1328 is positioned interiorly of the flange receiver wall 1326 to form a spring chamber 1330 therebetween. A shuttle retaining wall 1332 extends from the side wall 1296 and is spaced from and generally parallel to the latch retaining wall 1314, and cooperates with the latch retaining wall 1314 to guide the movement of the shuttle 1268. A triangular front trigger opening 1334 is formed in the side wall 1296, and opens into a toggle chamber 1336. Proximal of the toggle chamber 1336, the top wall 1298 is provided with a toggle pivot retainer 1338 for rotatably mounting the toggle piece 1276.

Regarding operational support of the elements comprising the cannula operation assembly 1254, the right housing shell 1246 comprises an upper spoon cannula carriage rail 1340 and a lower spoon cannula carriage rail 1342 in parallel, spaced-apart juxtaposition extending inwardly from the side wall 1296. The spoon cannula carriage rails 1340, 1342 each terminate in a respective stop 1344, 1346. A hook retainer 1347 protrudes from the side wall 1296 above the upper rail 1340. A coring cannula carriage cradle 1348 comprises an upper cradle piece 1350 and a lower cradle piece 1352 extending inwardly from an intermediate location on the side wall 1296 and defining an arcuate, inward-facing surface. An upper helical drive member rail 1354 and a lower helical drive member rail 1356 extend inwardly from the side wall 1296 from the coring cannula carriage cradle 1348 to a proximal drive member retaining wall 1370 in parallel, spaced-apart juxtaposition to define a slot 1360. A pair of vertical ribs 1362, 1364 extend orthogonally from the upper rail 1354 to the lower rail 1356 and define a viewing window 1366 therebetween. A ramped surface 1358 protruding from the side wall 1296 extends towards each rib 1362, 1364. A driven member mounting chamber 1368 is defined near the distal end of the right housing shell 1246 by the proximal driven member retaining wall 1370 and a distal driven member retaining wall 1372. An aperture 1374 is formed in the distal driven member retaining wall 1372 to allow the cannula assembly 14 to pass therethough and extend through an opening 1376 formed in a nosepiece 1378 protruding from the distal wall 1304.

Regarding operational support of the elements comprising the sample size control assembly 1256, the right housing shell 1246 comprises a wheel slot 1380 formed in the bottom wall 1300 near the proximal end of the right housing shell 1246. The wheel slot 1380 opens into an adjuster chamber 1382 extending from the rear wall 1302 to an adjuster retaining wall 1384. A wing slot 1386 communicates with the adjuster chamber 1382 through the side wall 1296. A proximal mount 1388 is formed in the rear wall 1302 and a distal mount 1390 is formed in the retaining wall 1384. A stylet aperture 1392 is also formed in the retaining wall 1384, above the distal mount 1390.

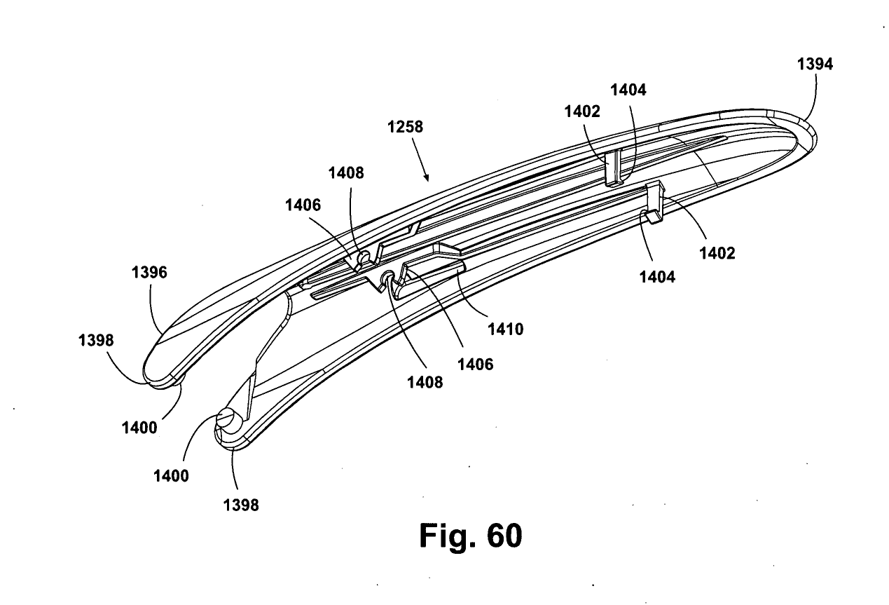

Referring to FIG. 60, the cocking lever 1258 is an elongated member having a proximal end 1394 and a distal end 1396. The distal end 1396 terminates in a pair of parallel, spaced-apart, inclined pivot extensions 1398, each of which terminates in an inwardly extending pivot boss 1400. The pivot extensions 1396 and pivot bosses 1400 are adapted for receipt in the pivot apertures 1322 with the housing shells 1244, 1246 in an assembled configuration. The cocking lever 1258 is adapted for pivotal movement about an axis coaxial with the pivot apertures 1322. A pair of opposed latch extensions 1402 extends orthogonally from the underside of the handle 126 at the proximal end 1394 thereof, each latch extension 1402 terminating in a hook 1404. A pair of parallel, spaced-apart swing arm supports 1406 extends from the underside of the handle 126 intermediate the proximal end 1394 and the distal end 1396. The swing arm supports 1406 are provided with pivot openings 1408 defining a rotational axis orthogonal to the longitudinal axis of the handle 126. On either side of the swing arm supports 1406 are a pair of pivot recesses 1410 extending into the underside of the cocking lever 1258.

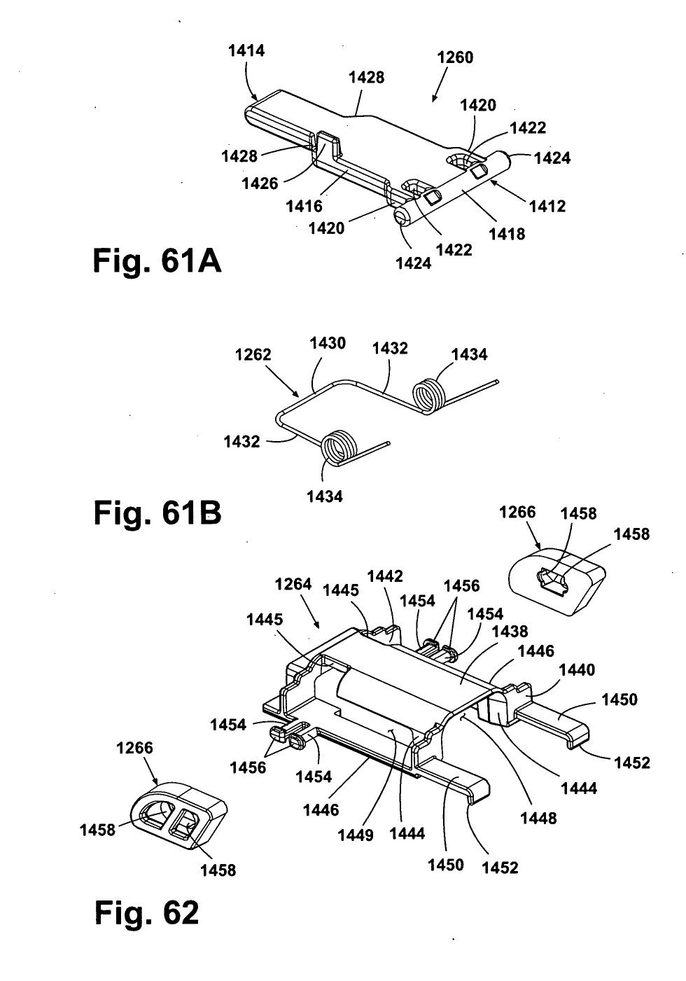

Referring to FIG. 61A, the swing arm 1260 is a generally flattened member having a pivot end 1412 and a sliding end 1414 interconnected by a center member 1416. The pivot end 1412 comprises a cylindrically shaped pivot rod 1418 orthogonal to the longitudinal axis of the center member 1416 and extending laterally away from the center member 1416. A pair of curved buttresses 1420 extend from the pivot rod 1418 to join the center member 1416 at an intermediate region thereof, to define a pair of spaced-apart openings 1422. The sections of the pivot rod 1418 adjacent the openings 1422 are adapted for insertion into the pivot openings 1408 of the swing arm supports 1406 of the cocking lever 1258 for pivotal mounting of the swing arm 1260 to the cocking lever 1258. The section of the pivot rod 1418 outside the openings 1422 form pivot rod ends 1424. The center member 1416 comprises a protrusion 1426, which is related to the manufacture of the swing arm 1260, extending normally from the surface of the center member 1416 and a pair of shoulders 1428 that narrow the center member 1416 where it joins the sliding end 1414.

Referring to FIG. 61B, the spring 1262 is a resilient, generally U-shaped member comprising a spring body 1430 having a pair of spaced spring arms 1432 formed orthogonally to the spring body 1430, each having a coiled end 1434 sized for insertion of the pivot rod ends 1424.

Referring to FIG. 62, the latch 1244 is a carriage-like body comprising a rectilinear frame portion having an upper center wall 1438 joining a pair of orthogonally-depending, spaced-apart distal and proximal walls 1440, 1442 and a pair of orthogonally-depending, spaced-apart sidewalls 1444. The center wall 1438 comprises two latch recesses 1445 adjacent the proximal wall. Two lower walls 1446 flank the upper center wall 1438 and are joined to the center wall 1438 by the distal wall 1440, proximal wall 1442 and sidewalls 1444 to define a rectilinear shuttle cavity 1448. Each lower wall 1446 is spaced from the upper center wall 1438 to form a latch channel 1449 therebetween in communication with the latch recess 1445. Extending away from the distal wall 1440 are pair of flex arms 1450, each flex arm 1450 terminating in a downwardly depending tooth 1452. A pair of latch button arms 1454 extend from each lower flanking wall 1446, in a direction away from the center wall 1438, and terminate in a latch button hook 1456. The latch buttons 1266 are provided with a pair of hook openings 1458 that receive the latch button hooks 1456 to mount the latch buttons 1266 to the latch 1244.

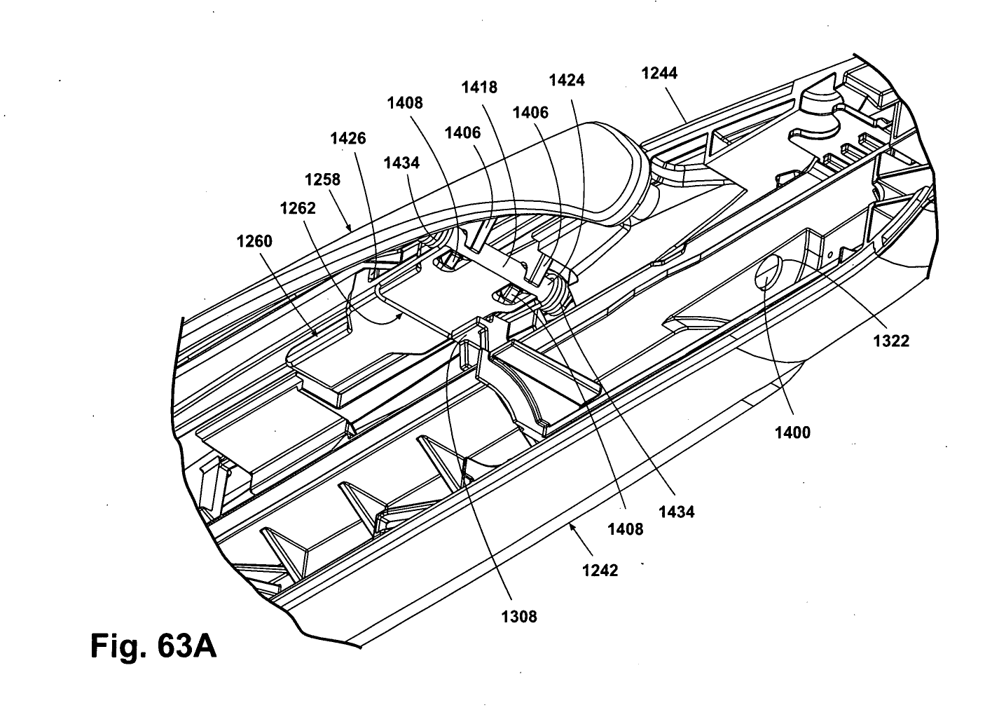

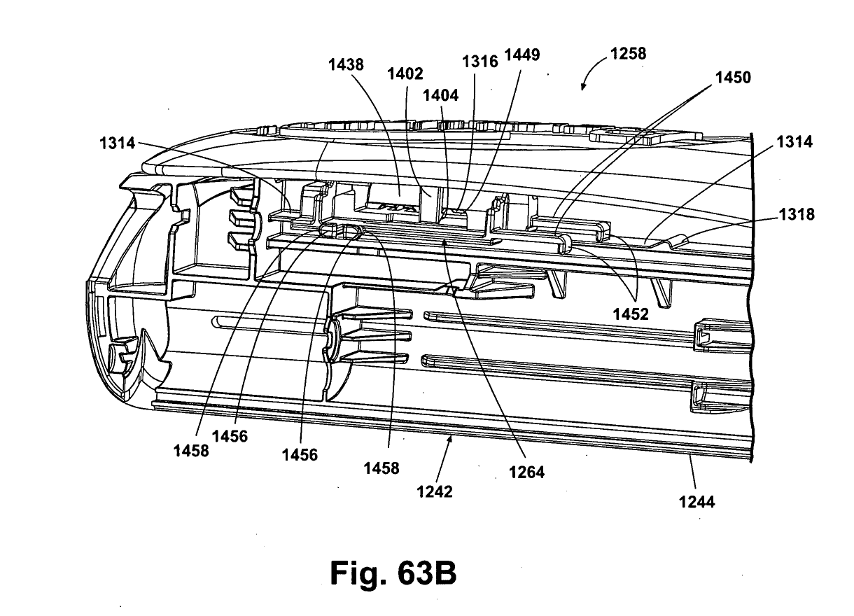

The assembled cocking handle assembly 1238 will now be described with reference to FIGS. 63A-B, in which elements of the biopsy gun 1230 unrelated to the cocking handle assembly 1238, particularly the right housing shell 1246, will not be shown in order to facilitate a clear understanding of the assembly of the cocking handle assembly 1238. It should be noted that the mounting of the cocking handle assembly 1238 to the right housing shell 1246 is substantially identical to as the left housing shell. Thus, the description of the mounting to the left housing shell will apply to the right housing shell.

The cocking lever 1258 can be installed to the housing 1242 by inserting the pivot bosses 1400 into the pivot apertures 1322. This is done with the housing shells 1244, 1246 assembled into the housing 1242. The spring 1262 is mounted to the swing arm 1260 by inserting the pivot rod ends 1424 into the coiled ends 1434 of the spring 1262. The swing arm 1260 is then pivotally mounted to the cocking lever 1258 via the pivot rod 1418. The swing arm supports 1406 are snap-fit to the swing arm 1260 by inserting the pivot rod 1418 into the pivot openings 1408, such that the pivot rod ends 1424 are received within the pivot recesses 1410. The swing arm stop 1308 prevents the swing arm 1260 from pivoting below a generally horizontal orientation when the cocking lever 1258 is in the closed or latched position. The latch 1264 is supported on the latch retaining wall 1314, with the latch button arms 1454 protruding through the latch slots 1316 and the teeth 1452 of the flex arms 1450 proximal of the latch protrusions 1318. The latch buttons 1266 are mounted to the latch 1264 by inserting the latch button hooks 1456 through the hook openings 1458. The latch extensions 1402 on the cocking lever 1258 extend through the latch opening 1310 on either side of the upper center wall 1438, such that the hooks 1404 are received in the latch channels 1449.

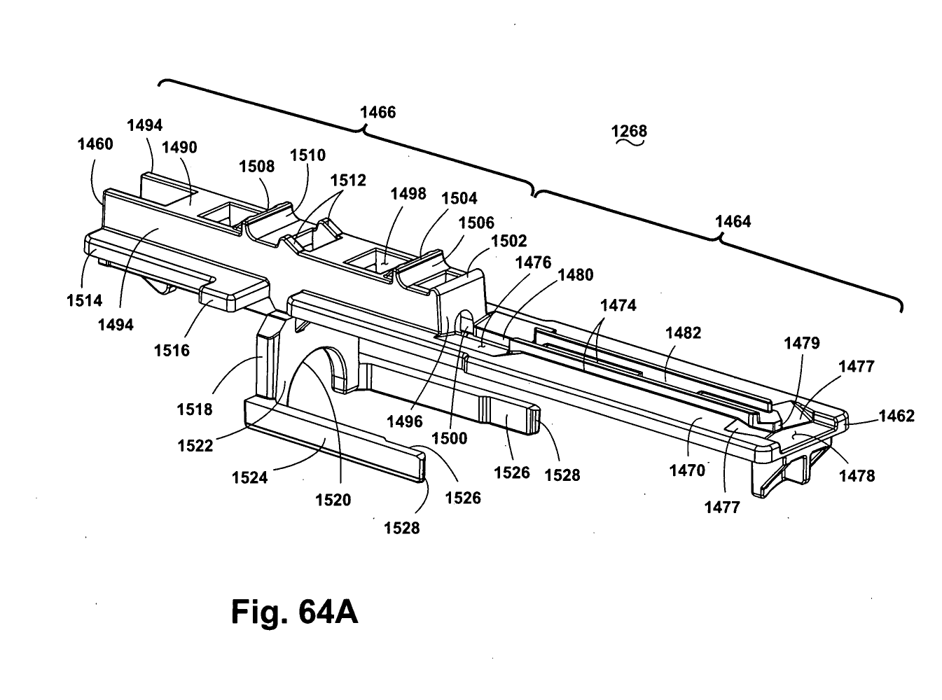

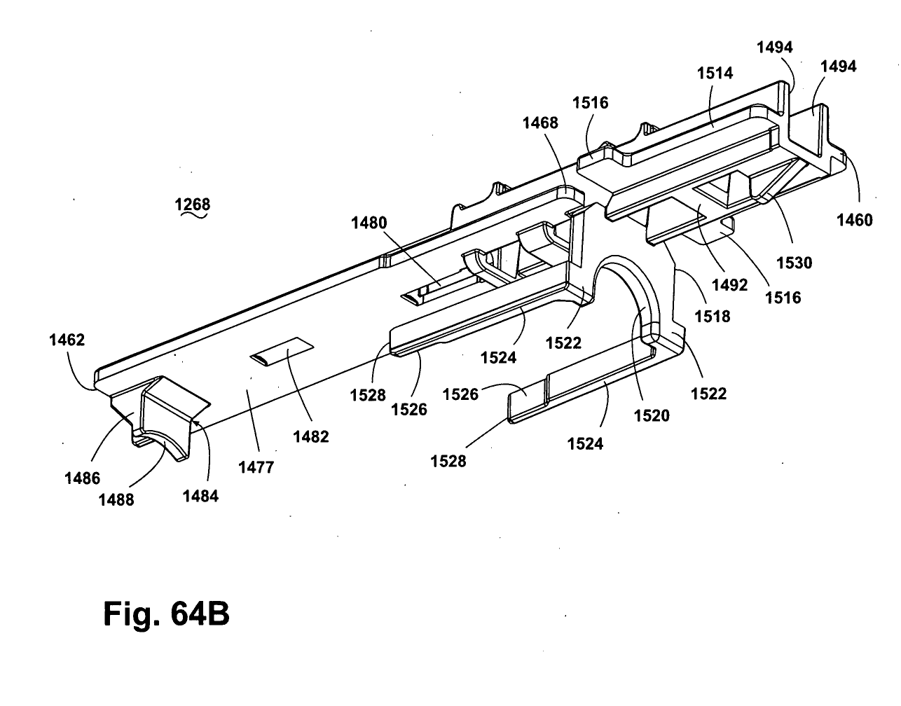

Referring to FIGS. 64A-B, the shuttle 1268 is an elongated carriage-like body having a proximal end 1460 and a distal end 1462, and comprising a plate portion 1464 and a box portion 1466. The plate portion 1464 extends from the distal end 1462 into cooperative registry with the box portion 1466, terminating in a plate proximal end 1468 at approximately the mid-line of the box portion 1466. The box portion 1466 extends from the plate portion 1464 to the proximal end 1460.

The plate portion 1464 is an elongated member having an upper surface 1470 and a lower surface 1472. A pair of spaced guide rails 1474 are formed on the upper surface 1470 and generally extend between a proximal depressed area 1476 and a distal depressed area 1478, which includes a pair of sloped cam surfaces 1477 formed on either side of a catch retainer 1479. A first pocket 1480 extends through the plate portion 1464 in the proximal depressed area 1476 and a second pocket 1482, formed distally of the first pocket 1480, extends through the plate portion 1464 between the guide rails 1474. Depending from the lower surface 1472 near the distal end 1462 is a cradle 1484 comprising a cradle wall 1486 terminating in an arcuate surface 1488 opening away from the lower surface 1472.

The box portion 1466 is an elongated, rectilinear structure comprising a top wall 1490 parallel to and spaced away from the upper surface 1470 of the plate portion 1464, and a bottom wall 1492 parallel to and spaced away from the top wall 1490 parallel to the plate portion 1464. A pair of parallel, spaced-apart sidewalls 1494 depends orthogonally from the top wall 1490 for connection, in part, with the plate portion 1464 and, in part, with the bottom wall 1492. An inner end wall 1496 extends orthogonally away from the upper surface 1470 of the plate portion 1464 to join the sidewalls 1494 to enclose a distal end of the box portion 1466. The walls 1490-1496 define an elongated, rectilinear chamber 1498. A semicircular opening 1500 extends through the inner end wall 1496 in communication with the chamber 1498.

A boss 1502 extends away from the top wall 1490, along the inner end wall 1496. An inner bearing wall 1504 extends orthogonally away from the top wall 1490, intermediate the boss 1502 and the proximal end 1460 and is provided with an arcuate surface 1506 on the distal side of the wall 1504. An outer bearing wall 1508 extends orthogonally away from the top wall 1490 intermediate the inner bearing wall 1504 and the proximal end 1460. The outer bearing wall 1508 is provided with an arcuate surface 1510 on the distal side of the wall 1508. A pair of triangular protrusions 1512 extend orthogonally away from the top wall 1490, intermediate the inner and outer bearing walls 1504, 1508. An elongated side rib 1514 extends laterally away from each side wall 1494 coplanar with the plate portion 1464. An end boss 1516 extends laterally away from the distal end of each side rib 1514.

Depending orthogonally from the bottom wall 1492 proximally of the plate proximal end 1468 is an intermediate wall 1518 inset with a cradle 1520 opening ventrally away from the box portion 1466. The lateral sides of the cradle 1520 define a pair of downwardly-depending wings 1522. Extending distally from a lower lateral portion of each wing 1522 is a cantilevered lower rail 1524. Each lower rail 1524 terminates in an inset end portion 1526 having a tapered tip 1528. Depending from the proximal end of the bottom wall 1492 is a plate-like fin 1530 disposed parallel to the longitudinal axis of the shuttle 1268.

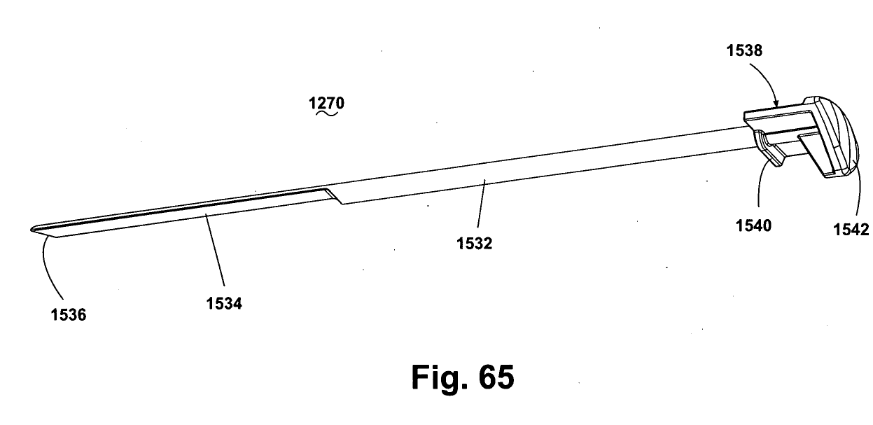

Referring to FIG. 65, the trigger plunger 1270 is an elongated, somewhat nail-shaped member comprising a cylindrical rod 1532 terminating at one end in a an inset portion 1534 forming a hemispherical rod section having an angled tip 1536. A trigger portion 1538 is connected to the end of the cylindrical rod 1532 opposite the inset portion 1534 and has a mounting flange 1540 adjacent the cylindrical rod 1532 and a protuberance serving as a rear trigger button 1542 to fire the biopsy gun 1230.

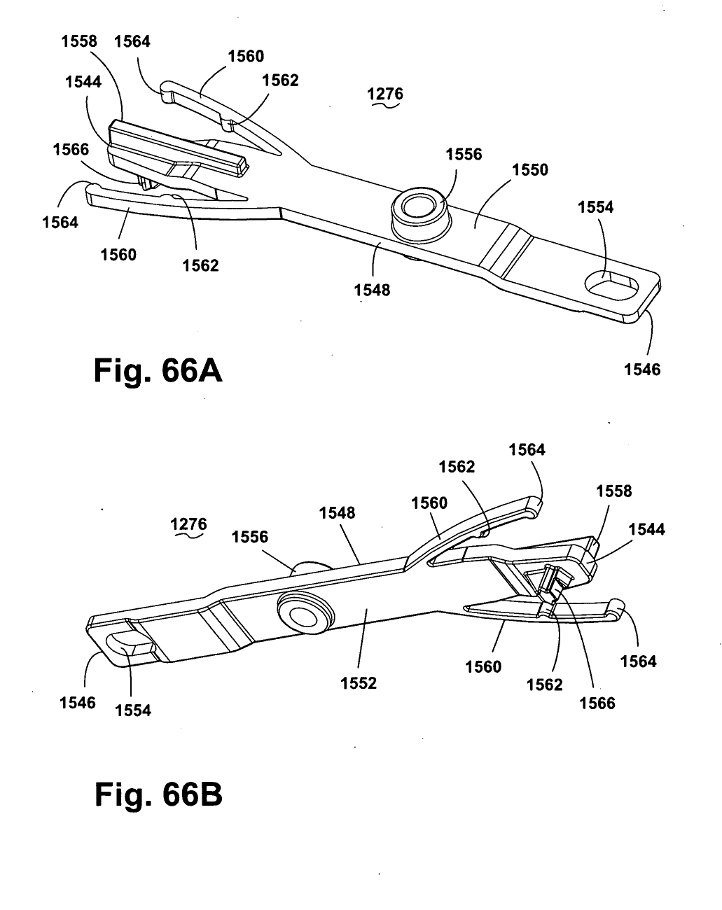

Referring to FIGS. 66A-B, the toggle piece 1276 is an elongated member having a proximal end 1544 and a distal end 1546 and comprises a flat body 1548 having an upper surface 1550 and a lower surface 1552. A toggle button opening 1554 extends through the flat body 1548 near the distal end 1546. A circular pivot boss 1556 extends orthogonally from the upper surface 1550 near the midpoint of the flat body 1548. Extending from the upper surface 1550 adjacent the proximal end 1544 is a rectilinear boss 1558 disposed parallel to the longitudinal axis of the toggle piece 1276. A pair of spring arms 1560 branch off from the flat body 1548 at an acute angle in the area of the rectilinear boss 1558 and comprise a first spacer 1562 formed on an intermediate portion of the spring arms 1560 and a second spacer 1564 formed on the terminal end of the spring arms 1560. A catch 1566 depends orthogonally from the lower surface 1552, in the region of the flat body 1548 between the spring arms 1560.

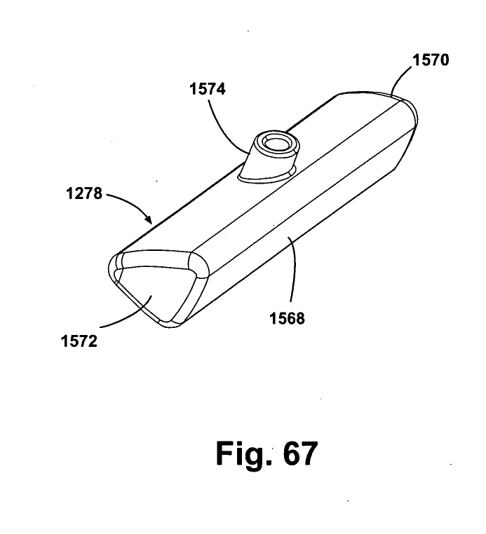

Referring to FIG. 67, the toggle button 1278 comprises an elongated triangular shaft 1568 having a first end serving as a left side trigger button 1570 and a second end serving as a right side trigger button 1572, either of which may fire the biopsy gun 1230. A circular pivot boss 1574 extends from the triangular shaft 1568 for receipt within the toggle button opening 1554 on the toggle piece 1276.

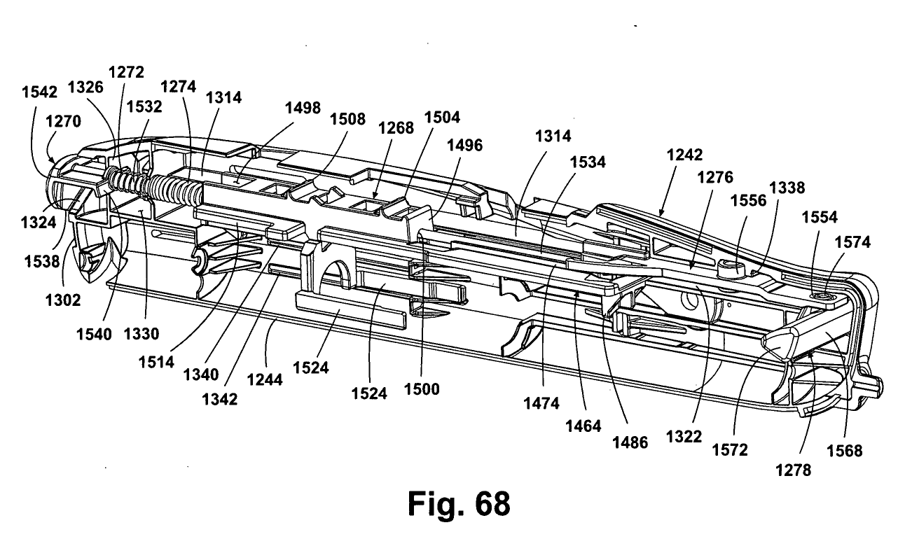

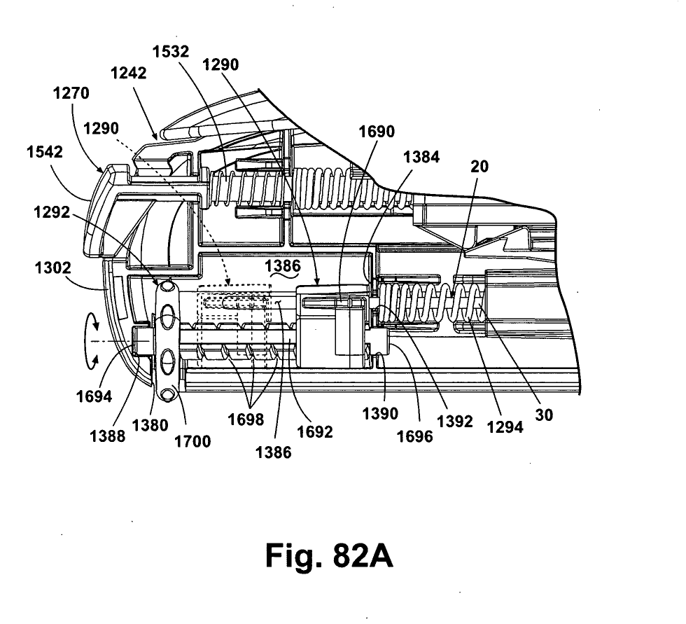

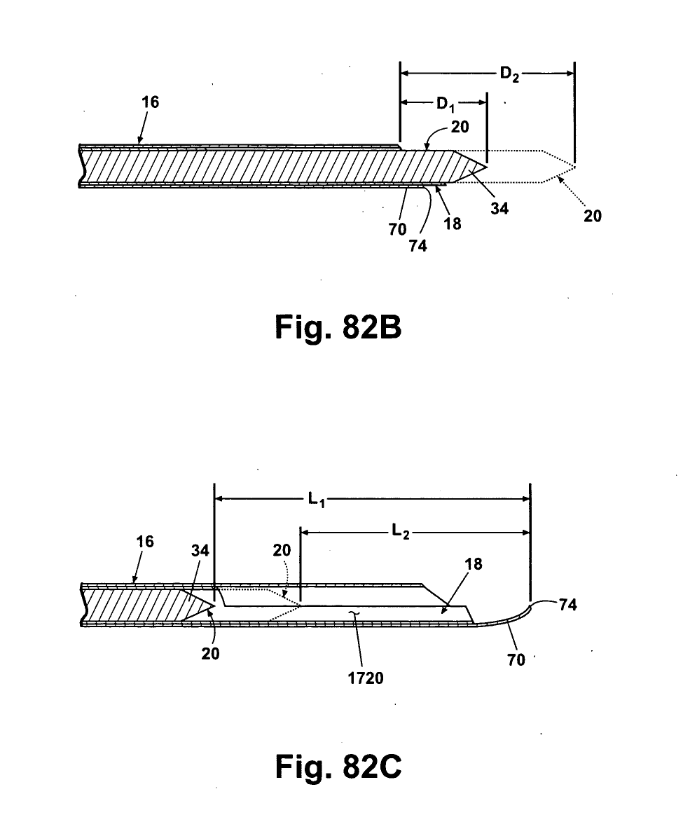

The assembled trigger assembly 1252 will now be described with reference to FIG. 68 in which elements of the biopsy gun 1230 unrelated to the trigger assembly 1252, particularly the right housing shell 1246, will not be shown in order to facilitate a clear understanding of the assembly of the trigger assembly 1252. The shuttle 1268 is mounted to the housing 1242 such that it is axially moveable relative to the housing 1242, with the sides ribs 1514 and the edges of the plate portion 1464 received between the latch retaining wall 1314 and the shuttle retaining wall 1332 and the lower rails 1524 received between the spoon cannula carriage rails 1340, 1342. The trigger plunger 1270 is mounted to the housing 1242 such that it is axially moveable relative to the housing 1242. The trigger portion 1538 is inserted into the flange receiver wall 1326 such that the mounting flange 1540 is received on the side of the wall 1326 within the spring chamber 1330 and the rear trigger button 1542 projects through the rear trigger openings 1324 in the proximal wall 1302 so that it is accessible to the user. The cylindrical rod 1532 passes through the chamber 1498 of the shuttle 1268 and extends through the semicircular opening 1500. The inset portion 1534 of the cylindrical rod 1532 is selectively received between the guide rails 1474. The first spring 1272 is mounted on the cylindrical rod 1532 of the trigger plunger 1270 within the spring chamber 1330 and biases the trigger plunger 1270 proximally. The second spring 1274 is mounted on the cylindrical rod 1532 of the trigger plunger 1270 between the spring retaining wall 1328 and the inner end wall 1496 of the shuttle 1268 to bias the shuttle 1268 distally. The toggle button 1278 mounted to the toggle piece 1276 by inserting the pivot boss 1574 of the toggle button 1278 into the toggle button opening 1554. The pivot boss 1556 of the toggle piece 1276 received by the toggle pivot retainer 1338 to rotatably mount the toggle piece 1276 to the housing 1242. With the toggle piece 1276 mounted to the housing 1242, the ends of the triangular shaft 1568 will project through the front trigger openings 1334 (FIG. 82) so that the left and right side trigger buttons 1570, 1572 are accessible to the user.

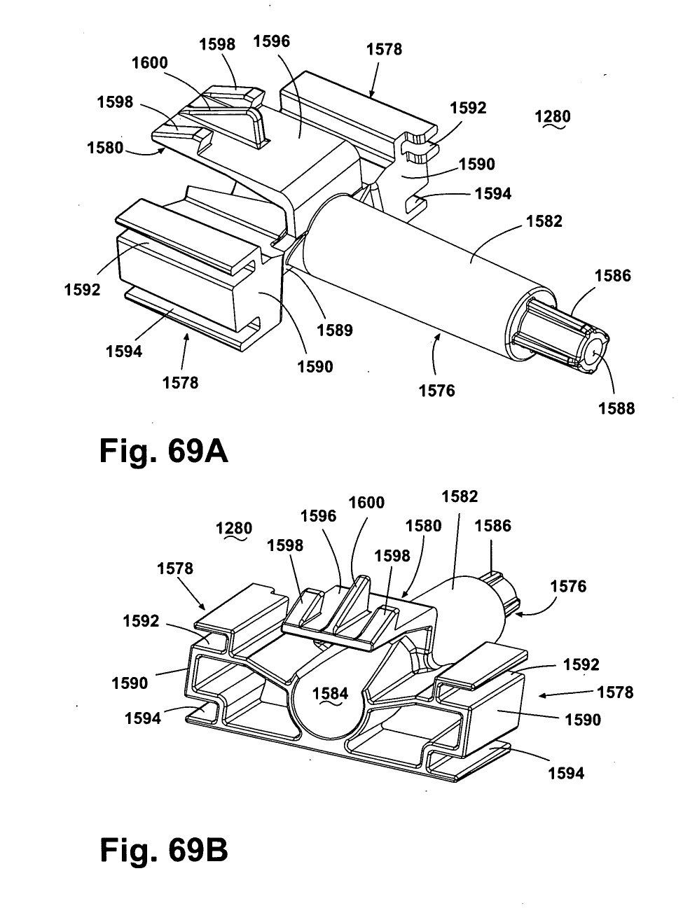

Referring to FIGS. 69A-B, the spoon cannula carriage 1280 comprises a barrel portion 1576, a pair of wing portions 1578, and a hook portion 1580. The barrel portion 1576 is an elongated, cylindrically-shaped body comprising an annular wall 1582 defining a barrel chamber 1584. The barrel portion 1576 terminates in a nosepiece 1586 having an aperture 1588 extending coaxially therethrough to communicate with the barrel chamber 1584. The proximal end of the spoon cannula 18 is received in the aperture 1588 for fixed attachment to the spoon cannula carriage 1280.

The barrel portion 1576 is joined to the wing portions 1578 by a canted face 1589. Each wing portion 1578 comprises a beam 1590, extending diametrically outwardly from a proximal end of the barrel portion 1576. Each beam 1590 is provided with a pair of spaced, elongated rectilinear slots parallel to the longitudinal axis of the barrel portion 1576; an upper end slot 1592 at an upper portion of the beam 1590 and a lower end slot 1594 at a lower portion of the beam 1590

The hook portion 1580 comprises an elongated member extending longitudinally away from a proximal end of the barrel portion 1576, disposed 90° from each wing portion 1578, parallel to the longitudinal axis of the barrel portion 1576. The hook portion 1580 comprises a strap-like resilient arm 1596 attached to the barrel portion 1576 in cantilevered fashion, terminating in a pair of upwardly-disposed hooks 1598. Extending from the resilient arm 1596 between the hooks 1598 is an upwardly-disposed, plate-like fin 1600 aligned with the longitudinal axis of the barrel portion 1576. The barrel portion 1576 is adapted for slidable registry with the cradle 1520 of the shuttle 1268, and the fin 1600 is adapted for engagement with the fin 1530.

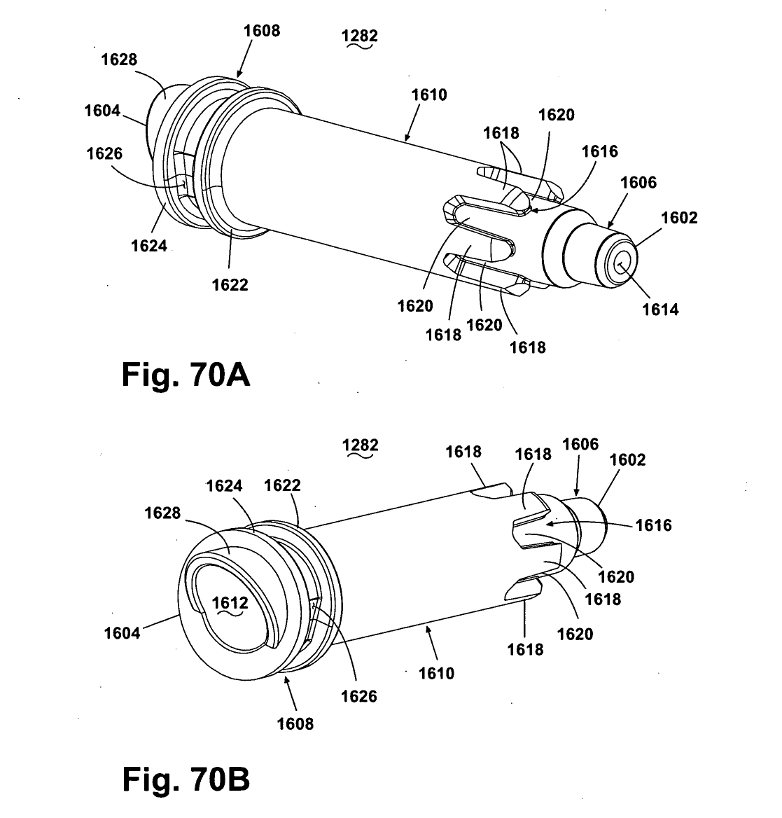

Referring to FIGS. 70A-B, the coring cannula carriage 1282 is an elongated, generally cylindrically-shaped body comprising a distal end 1602 and a proximal end 1604, and comprising a nosepiece 1606 at the distal end 1602, a flange portion 1608 at the proximal end 1604, and a barrel portion 1610 intermediate the nosepiece 1606 and the flange portion 1608. A carriage chamber 1612 extends through the coring cannula carriage 1282 from the distal end 1602 to the proximal end 1604. The barrel portion 1610 terminates at its intersection with the nosepiece 1606 in a circumferential toothed edge 1616 comprising a radial array of alternating projections 1618 and cavities 1620, which effectively form a gear. The flange portion 1608 comprises a pair of spaced circumferential flanges, including an inner flange 1622 and an outer flange 1624 defining a space 1626 therebetween. A semicircular canted wall 1628 extends proximally from the outer flange 1624 and is angled with respect to the longitudinal axis of the coring cannula carriage 1282. The nosepiece 1606 comprises an aperture 1614 coaxially extending therethrough in communication with the carriage chamber 1612. The aperture 1614 is adapted for fixed insertion of the coring cannula 16 so that the coring cannula 16 extends distally from the distal end 1602.

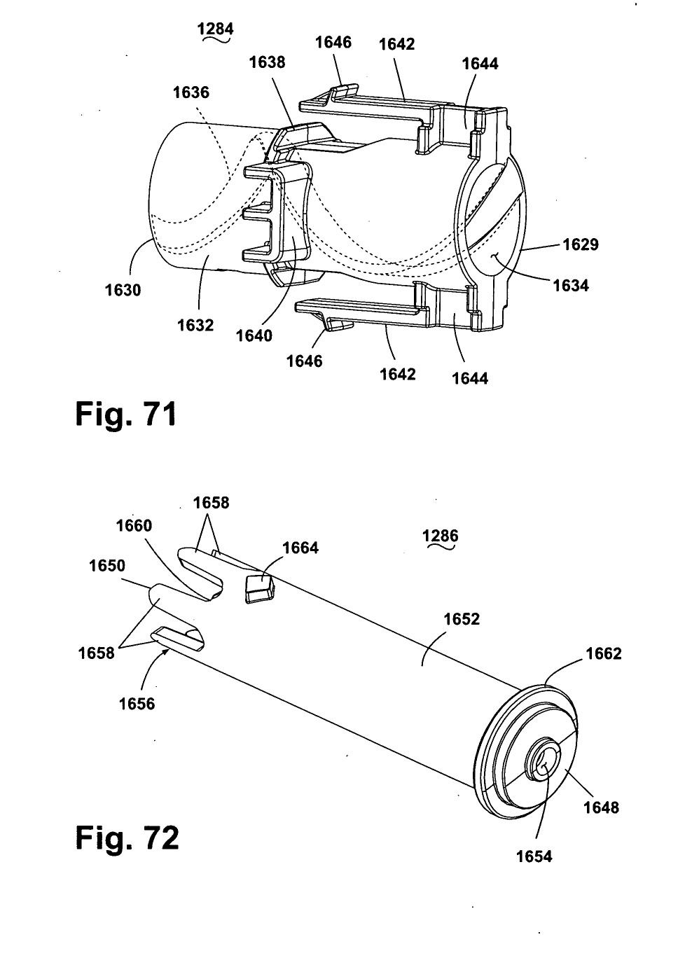

Referring to FIG. 71, the helical drive member 1284 is an elongated, generally annular body having a distal end 1629 and a proximal end 1630, and comprising an annular wall 1632 defining a cylindrical chamber 1634 open at both ends 1629, 1630. A helical channelway 1636 extends along the interior of the annular wall 1632 from the distal end 1629 to the proximal end 1630. A rib 1638 extends around an outer surface of the annular wall 1632 at approximately the mid-section of the annular wall 1362 and joins with a stop protrusion 1640 extending outwardly from the annular wall 1632. A pair of diametrically-opposed resilient arms 1642 extends longitudinally along the outer surface of the annular wall 1632 from the distal end 1629 toward the proximal end 1630. The resilient arms 1642 are attached in cantilevered fashion to the annular wall 1632 through a pair of mounting blocks 1644 at the distal end 1629. The resilient arms 1642 terminate in a pair of radially outwardly-extending hooks 1646, respectively.

Referring to FIG. 72, the rotating driven member 1286 is an elongated, generally annular body having a distal end 1648 and a proximal end 1650, and comprising an annular wall 1652 defining a cylindrical aperture 1654 extending between both ends 1648, 1650. The annular wall 1652 terminates at the proximal end 1650 in a circumferential toothed edge 1656 comprising a radial array of alternating projections 1658 and cavities 1660 that effectively form a gear that can mesh with the toothed edge 1616 on the coring cannula carriage 1282. The annular wall 1652 transitions at the distal end 1648 to a circular flange 1662 having a diameter somewhat greater than the diameter of the annular wall 1652. A square boss 1664 extends radially away from the annular wall 1652 adjacent the proximal end 1650, and is adapted for slidable registry with the helical channelway 1636. The annular wall 1652 is adapted for slidable insertion in the chamber 1634 of the helical drive member 1284, with the boss 1664 received in the channelway 1636. The aperture 1654 at the proximal end 1650 is adapted to receive the nosepiece 1606 of the coring cannula carriage 1282.

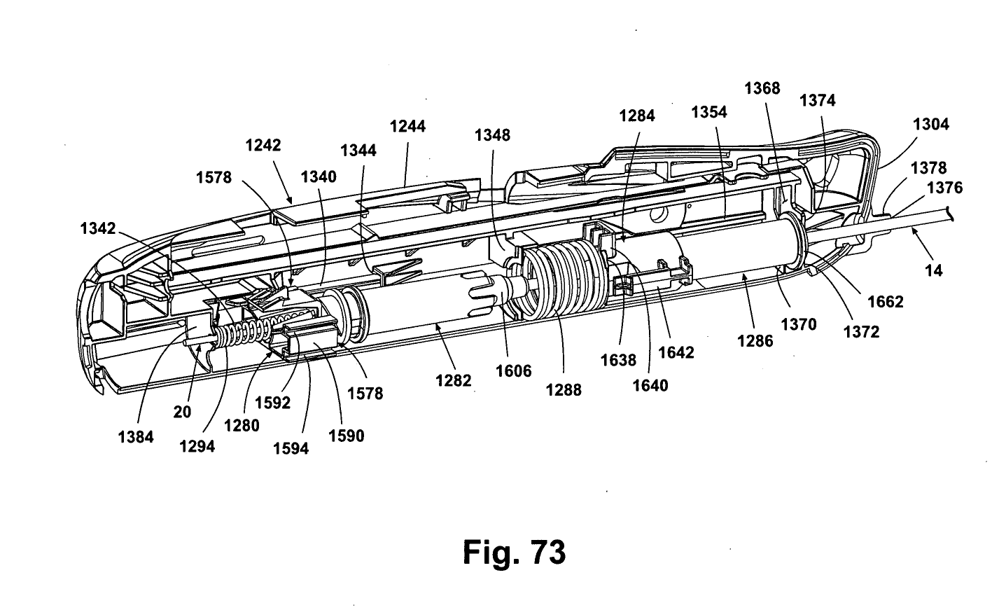

The assembled cannula operation assembly 1254 will now be described with reference to FIGS. 59 and 73, in which elements of the biopsy gun 1230 unrelated to the cannula operation assembly 1254, particularly the right housing shell 1246, will not be shown in order to facilitate a clear understanding of the assembly of the cannula operation assembly 1254. The spoon cannula carriage 1280 is slidably supported within the housing 1242 through receipt of the spoon cannula carriage rails 1340, 1342 by the wing portions 1578, with the upper spoon cannula carriage rail 1340 received within the upper end slot 1592 and the lower spoon cannula carriage rail 1342 received within the lower end slot 1594. The spoon cannula carriage 1280 is axially moveable along the spoon cannula carriage rails 1340, 1342, with the extent of its distal movement limited by the stops 1344, 1346. The coring cannula carriage 1282 is positioned within the housing 1242 distally of and coaxially with the spoon cannula carriage 1280, with the nosepiece 1606 protruding through the coring cannula carriage cradle 1348. The helical drive member 1284 is mounted to the housing 1242 distally of the coring cannula carriage 1282, with the resilient arms 1642 received within the slot 1360 between the helical drive member rails 1354, 1356. The rotating driven member 1286 is mounted to the housing 1242 distally of the helical drive member 1284, with the circular flange 1662 received within the driven member mounting chamber 1368 such that the cylindrical aperture 1654 (FIG. 72) of the rotating driven member 1286 is coaxially aligned with the aperture 1374 in the distal driven member retaining wall 1372. The spring 1288 is positioned between the coring cannula carriage cradle 1348 and the rib 1638 of the helical drive member 1284. The spring 1294 is positioned between the adjuster retaining wall 1384 and the coring cannula carriage 1280, whereby a portion of the spring 1294 is received within the barrel chamber 1584.

The cannula assembly 14, which includes the coring cannula 16 extending from the coring cannula carriage 1282, the spoon cannula 18 extending from the spoon cannula carriage 1280, and the stylet 20 extending from the stylet carriage 1290, passes through the cylindrical chamber 1634 of the helical drive member 1284 and the cylindrical aperture 1654 of the rotating driven member 1286 to protrude distally of the housing 1242 through the opening 1374 in the nosepiece 1378 protruding from the distal wall 1304.

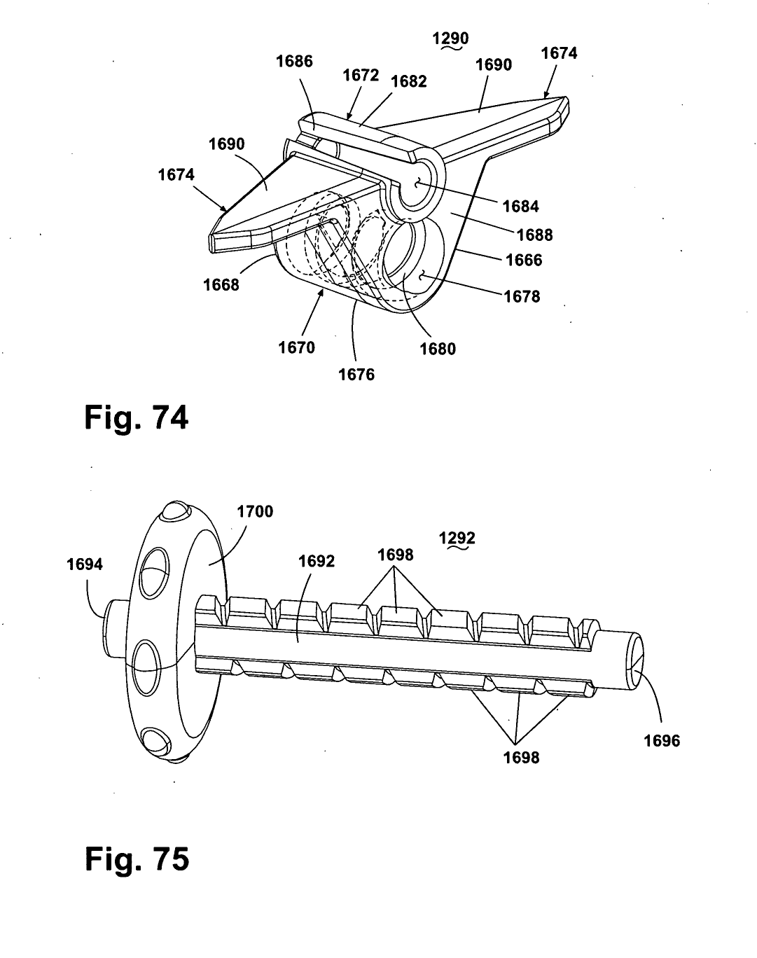

Referring to FIG. 74, the stylet carriage 1290 has a distal end 1666 and a proximal end 1668 and comprises a lower barrel portion 1670, an upper barrel portion 1672 and a pair of wing portions 1674. The barrel portion 1670 comprises an annular wall 1676 defining a cylindrical chamber 1678 open at both ends 1666, 1668. A set of screw threads 1680 extends along the interior of the annular wall 1676 from the distal end 1666 to the proximal end 1668. The upper barrel portion 1672 is generally aligned with the lower barrel portion 1670 and comprises an annular wall 1682 defining a stylet chamber 1684 that is open at the distal end 1666 and closed at the proximal end 1668. The annular wall 1682 has an open section 1686 extending from the proximal end 1668 to the distal end 1666 that allows the stylet chamber 1684 to expand in diameter towards the distal end 1666. The wing portions 1674 each comprises a triangular brace 1688 extending from the annular wall 1676 at the distal end 1666 to support an elongated wing 1690.

Referring to FIG. 75, the adjustment member 1292 is an elongated, somewhat nail-shaped member comprising cylindrical shaft 1692 terminating in a proximal mounting end 1694 and a distal mounting end 1696, with screw threads 1698 formed on the cylindrical shaft 1692 between the proximal and distal mounting ends 1694, 1696. A circular adjustment wheel 1700 is positioned coaxially on the cylindrical shaft 1692, near the proximal mounting end 1694, and may be integrally formed therewith.

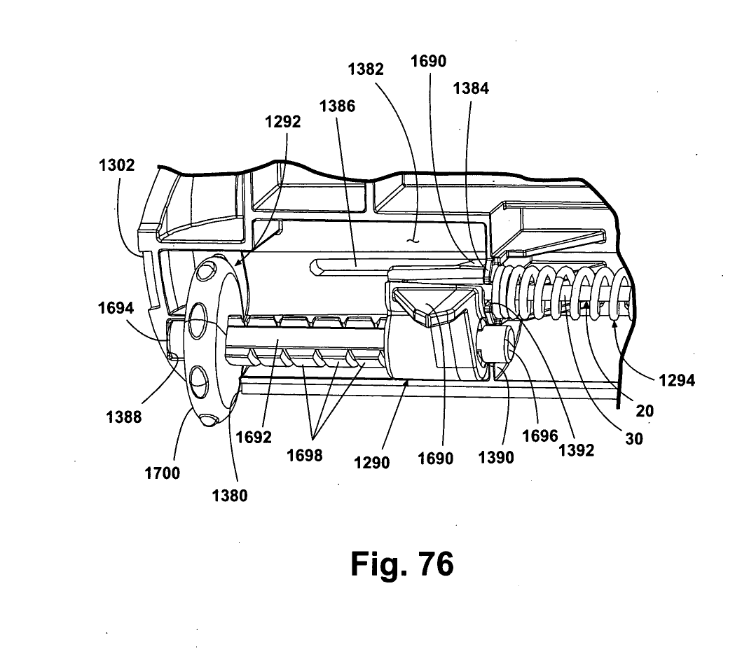

The assembled sample size control assembly 1256 will now be described with reference to FIG. 76 in which elements of the biopsy gun 1230 unrelated to the sample size control assembly 1256, particularly the right housing shell 1246, will not be shown in order to facilitate a clear understanding of the assembly of the sample size control assembly 1256. The stylet carriage 1290 is movably mounted on the adjustment member 1292 by inserting the cylindrical shaft 1692 into the cylindrical chamber 1678 and rotating the stylet carriage 1290 relative to the adjustment member 1292 such that the screw threads 1680, 1698 engage each other. The adjustment member 1292 is then fixedly mounted within the housing 1242, with the proximal mounting end 1694 received within the proximal mount 1388, the distal mounting end 1696 received within the distal mount 1390, and the adjustment wheel 1700 projecting through the wheel slot 1380 such that at least a portion of the adjustment wheel 1700 is accessible to the user. The proximal end of the stylet 20 is received in the stylet chamber 1684 for fixed attachment to the stylet carriage 1290, with the stylet body 30 projecting distally though the stylet aperture 1392 in the adjuster retaining wall 1384. The wings 1690 of the stylet carriage 1290 will project through the wing slots 1386 to indicate the position of the stylet carriage, and therefore the stylet 20, relative to the housing 1242.

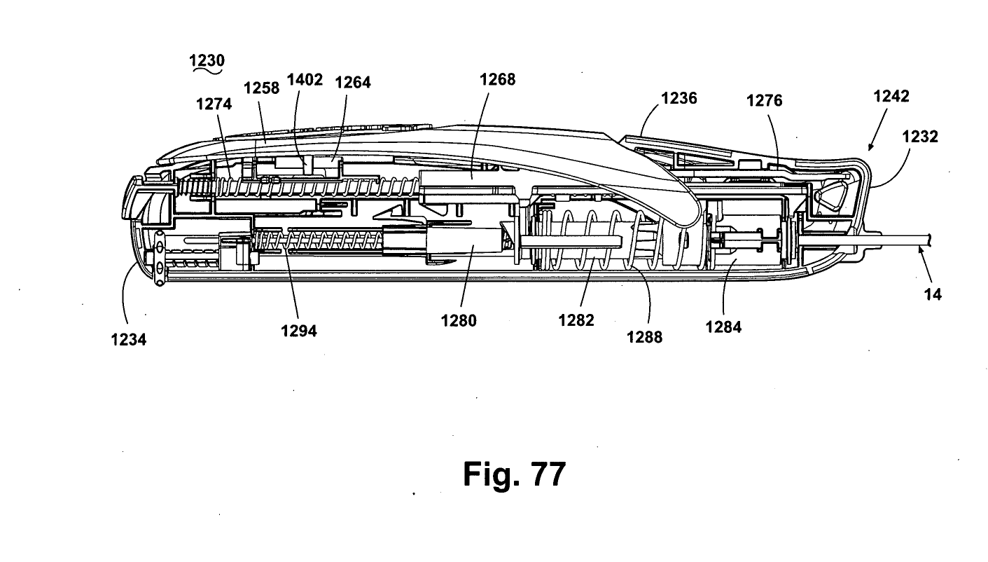

An exemplary description of the operation of the biopsy device 10 according to the third embodiment will now be described with reference to FIGS. 77-86. In the Figures, elements of the biopsy gun 1230, particularly the housing shells 1244, 1246, will be either removed or illustrated in phantom to facilitate a complete understanding of the operation of the gun 1230. The operation of the biopsy device 10 generally comprises the steps of: (i) cocking or arming the biopsy device 10; (ii) selecting the specimen size to be collected; (iii) firing the biopsy device 10 to collect a specimen; and (iv) retrieving the specimen from the biopsy device. It will be apparent to one of ordinary skill that the operation procedure can proceed in any logical order and is not limited to the sequence presented below. The following description is for illustrative purposes only and is not intended to limit the invention in any manner.

Referring to FIG. 77, the biopsy gun 1230 is initially in an uncocked condition, and is typically cocked prior to introducing the cannula assembly 14 into the tissue mass 22 (FIG. 1). In the uncocked condition, the biopsy gun 1230 is initially in a configuration with the cocking lever 1258 extending along the dorsal side 1236 of the housing 1242. The latch 1264 is urged towards the proximal end 1234 of the housing 1242 with the hooks 1404 of the latch extensions 1402 in cooperative registry with the latch channel 1449 to retain the cocking lever 1258 against the housing 1242. Under the influence of the springs 1274 and 1294, the shuttle 1268 and the two cannula carriages 1280, 1282 are respectively urged toward the distal end 1232 of the housing 1242. The shuttle 1268 is orientated relative to the toggle piece 1276 such that the catch 1566 is received in the first pocket 1480. Under the influence of the spring 1288, the helical drive member 1284 is urged toward the distal end 1232 of the housing 1242.

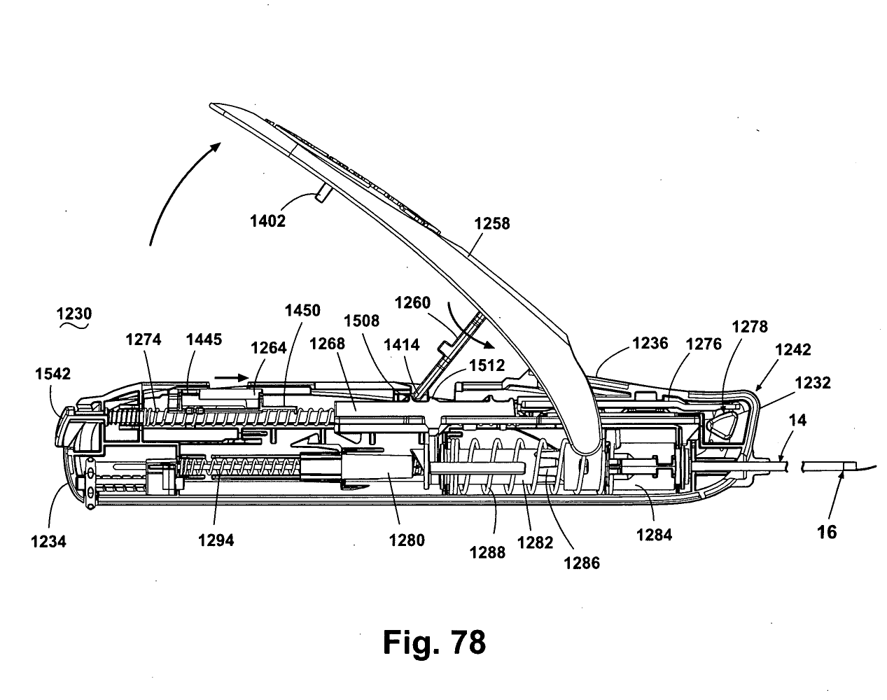

As illustrated in FIG. 78, the cocking lever 1258 is released by translating the latch 1264 distally by moving the latch buttons 1266 to the distal end of the latch slots 1316, which will translate the hooks 1404 to the latch recesses 1445, enabling clearance of the latch extensions 1402 from the latch 1264 (FIGS. 62 and 63B). The flex arms 1450 will move over the latch protrusions 1318 so that teeth 1452 are distal to the latch protrusions 1318 and thus prevents the latch 1264 from moving proximally and relatching the cocking lever 1258 before the biopsy gun 1230 is fully cocked or armed (FIG. 63B). The cocking lever 1258 will be urged into an open position by the spring 1262 (FIG. 63A), which will also move the swing arm 1260 to an oblique orientation to lodge the sliding end 1414 between the outer bearing wall 1508 and the triangular protrusions 1512 of the shuttle 1268.

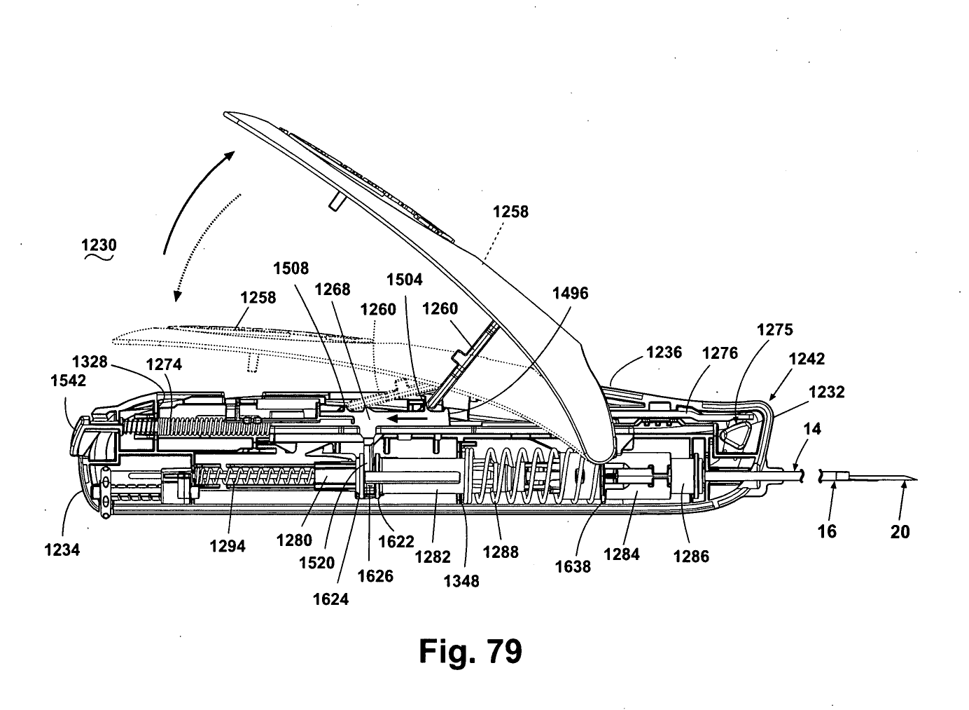

Referring to FIG. 79, depressing the cocking lever 1258 toward the housing 1242 a first time (as shown in phantom line) will drive the shuttle 1268 distally by virtue of the swing arm 1260 pressing against the arcuate surface 1510 (FIG. 64A) of the bearing wall 1508. This will urge the coring cannula carriage 1282 proximally due to the registry of the cradle 1520 on the shuttle 1268 with the space 1626 defined by the flanges 1622, 1624 of the coring cannula carriage 1282, thereby compressing the spring 1274 between the spring retaining wall 1328, and the inner end wall 1496 of the shuttle 1268. The coring cannula carriage 1282 will be drawn into a nesting arrangement with the spoon cannula carriage 1280, with the annular wall 1582 of the spoon cannula carriage 1280 received in the carriage chamber 1612 and the canted wall 1628 on the coring cannula carriage 1282 in contact with the canted face 1589 on the spoon cannula carriage 1280 (FIGS. 69A and 70B). The coring cannula 16 will be drawn over the stylet 20 and the spoon cannula 18 in a proximal direction. Upon release of the cocking lever 1258, the cocking lever 1258 will automatically return to the open position and the swing arm 1260 will move from outer bearing wall 1508 to inner bearing wall 1504.

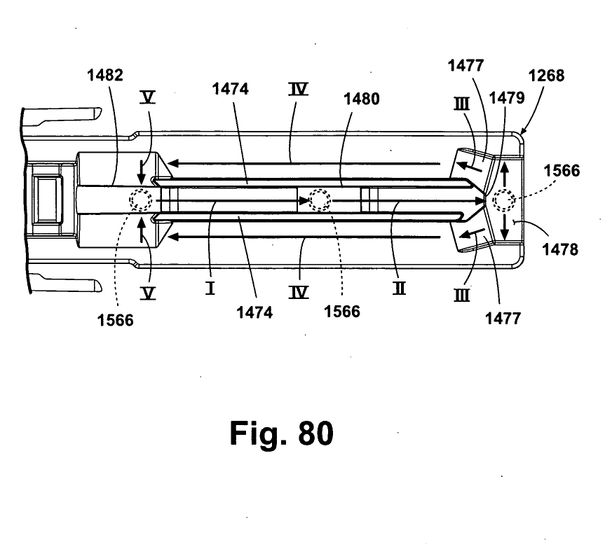

Referring to FIG. 80, which illustrates the relative movement of the shuttle 1268 and the toggle piece 1276 during operation of the biopsy gun 1230, during movement of the shuttle 1268 toward the proximal end 1234, the catch 1566 on the toggle piece 1276 will slide between the guide rails 1474 on the shuttle 1268 from the first pocket 1480 to the second pocket 1482, as indicated by arrow I. The shuttle 1268 will be prevented from returning to its initial uncocked position by the interference between the catch 1566 and the second pocket 1482.

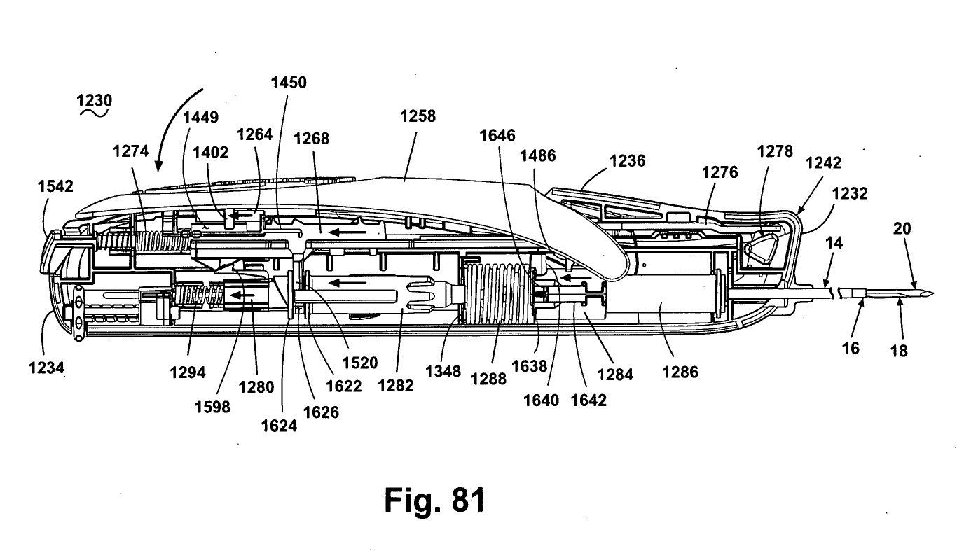

Referring to FIG. 79, depressing the cocking lever 1258 toward the housing 1242 a second time will again translate the shuttle 1268 proximally by virtue of the swing arm 1260 pressing against the arcuate surface 1506 (FIG. 64A) of the bearing wall 1504. Referring to FIG. 81, as the shuttle 1268 moves proximally, which compresses the spring 1274 further, the proximal end 1460 (FIG. 64B) will meet the proximal wall 1442 (FIG. 62) of the latch 1264, thus forcing the flex arms 1450 over the latch protrusions 1318 and urging the latch 1264 proximally in the latch slots 1316 (FIG. 63B). The cocking lever 1258 is now constrained in the closed position by virtue of the latch extensions 1402 being received in the latch channels 1449 of the latch 1264, without the necessity of manually resetting the position of the latch 1264.

The spoon cannula carriage 1280 and the coring cannula carriage 1282 will also be translated proximally by the shuttle 1268 as a result of the cradle 1520 being received on the coring cannula carriage 1282 and the nested arrangement of the spoon cannula carriage 1280 and the coring cannula carriage 1282. This will bring the hooks 1598 extending from the spoon cannula carriage 1280 into an interference engagement with the hook retainers 1347 (FIG. 59) formed on the housing shells 1244, 1246. The spring 1294 will also be fully compressed.

At the same time, the helical drive member 1284 will be translated proximally due to the registry of the cradle wall 1486 with the stop protrusion 1640, thereby compressing the spring 1288 between the rib 1638, and the carriage cradle 1348. The resilient arms 1642 of the helical drive member 1284 will be deflected radially inwardly by engagement of the hooks 1646 with the ramped surfaces 1358 leading to the ribs 1362 until the hooks 1646 lodge within the viewing windows 1366, thus preventing the helical drive member 1284 from moving distally under the influence of the spring 1288 (FIG. 59). The hooks 1646 can thus be used to indicate that the biopsy gun 1230 is fully cocked or armed by showing a distinctive color such as green in the viewing window 1366. In one contemplated configuration, the entire helical drive member 1284 can be colored green since it is only viewable in the viewing window 1366 after the second depression of the cocking lever 1258.

Further, in this configuration, the coring cannula 16 and the spoon cannula 18 will be fully retracted proximally, and the stylet 20 will extend distally of the coring cannula 16 and the spoon cannula 18, with the biopsy gun 1230 in a cocked and ready position for obtaining a biopsy sample. The cannula assembly 14 can be introduced into the tissue mass 22 and advanced to the lesion 24 for recovery of the biopsy sample 26.

Referring to FIG. 80 as the shuttle 1268 translates proximally the second time, the catch 1566 on the toggle button 1278 will slide between the guide rails 1474 from the second pocket 1482 to the distal depressed area 1478, as indicated by arrow II, and into registry with the catch retainer 1479. The shuttle 1268 is thus retained at its proximal limit, with the spring 1274 fully compressed.

Referring to FIGS. 82A-C the specimen size to be collected can optionally be selected using the sample size control assembly 1256 after the biopsy device 10 is cocked. The adjustment wheel 1700 is rotated to move the stylet carriage 1290 linearly along the cylindrical shaft 1692 of the adjustment member 1292. The stylet carriage 1290 will be moved distally or proximally depending on the direction in which the adjustment wheel 1700 is rotated. Markings (not shown) can be provided on the outer surface of the housing 1242 adjacent the wing slot 1386 to indicate various specimen lengths that can be collected by the biopsy device 10, with wings 1690 indicating the specimen length that the biopsy gun 1230 is currently set to take. Because the stylet 20 is fixed to the stylet carriage 1290, the stylet 20 will move relative to the coring cannula 16 and the spoon cannula 18; however, the distal tip of the stylet 20 will remain distal of the distal end of the cannulae 16, 18 prior to actuating the biopsy gun 1230. The cannulae 16, 18 always moves the same distance when the biopsy gun 1230 is actuated, while the stylet 20 remains stationary; therefore, adjusting the stylet 20 relative to the cannulae 16, 18 affects the size of specimen that is collected. The distance D between the distal or penetration tip 34 of the stylet 20 and the distal end of the coring cannula 16, which can be set using the sample size control assembly 1256, is proportional to the specimen length the biopsy gun 1230 will collect. The space between the distal end of the coring cannula 16 and the penetration tip 34 of the stylet 20 when the biopsy gun 1230 has been fired forms a tissue chamber 1702 which receives the specimen. The length L of a collected specimen is generally equal to the length of the tissue chamber 1702. Therefore, the farther the penetration tip 34 projects from the distal end of the coring cannula 16 prior to actuating the biopsy gun 1230, the smaller the collected specimen, and vice versa. For example, as illustrated in FIGS. 82B-C, setting the stylet 20 to the distance D₁ using the sample size control assembly 1256 will result in a specimen roughly the length L₁. Setting the stylet 20 to the distance D₂, which is greater than D₁, will result in a specimen roughly the length L₂, which is less than length L₂.

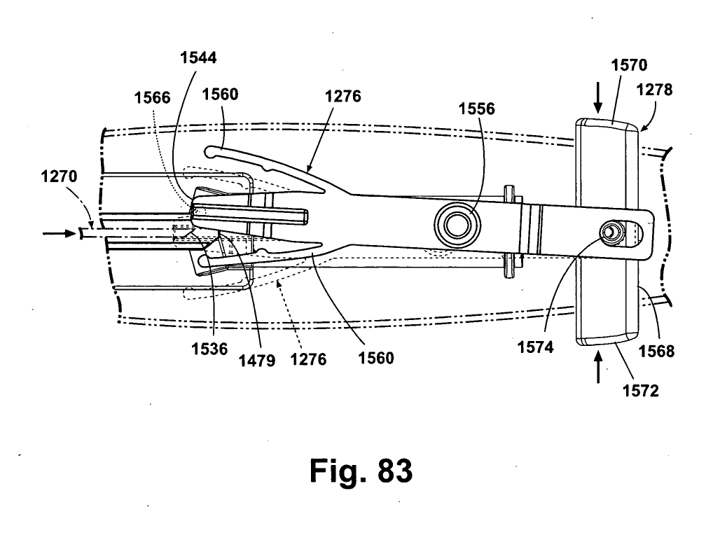

As previously described herein and illustrated in FIG. 9A, with the biopsy gun 1230 in the cocked condition, the cannula assembly 14 is inserted into the tissue mass 22 so that the penetration tip 34 is adjacent the lesion 24. The biopsy gun 1230 is then fired for excision of a biopsy sample or specimen. Referring to FIG. 83, the biopsy gun 1230 can be actuated in one of two ways to obtain the biopsy sample 26. Both procedures pivot the toggle piece 1276 to dislodge the catch 1566 from engagement with the catch retainer 1479 of the shuttle 1268. In the first procedure, rear trigger button 1542 (FIG. 82A) is depressed toward the distal end 1232, causing the entire trigger plunger 1270 to move distally so that the angled tip 1536 braces against the proximal end 1544 of the toggle piece 1276. The pressure of the angled tip 1536 against the proximal end 1544 causes the toggle piece 1276 to pivot about an axis defined by the pivot boss 1556 so that the catch 1566 clears the catch retainer 1479. In the second procedure, either the left or right side trigger button 1570, 1572 are depressed laterally inwardly, which also pivots the toggle piece 1276 so that the catch 1566 clears the catch retainer 1479. Regardless of the way in to the biopsy gun is actuated, one the catch 1566 clears the catch retainer 1479, the sequence of operations is the same.

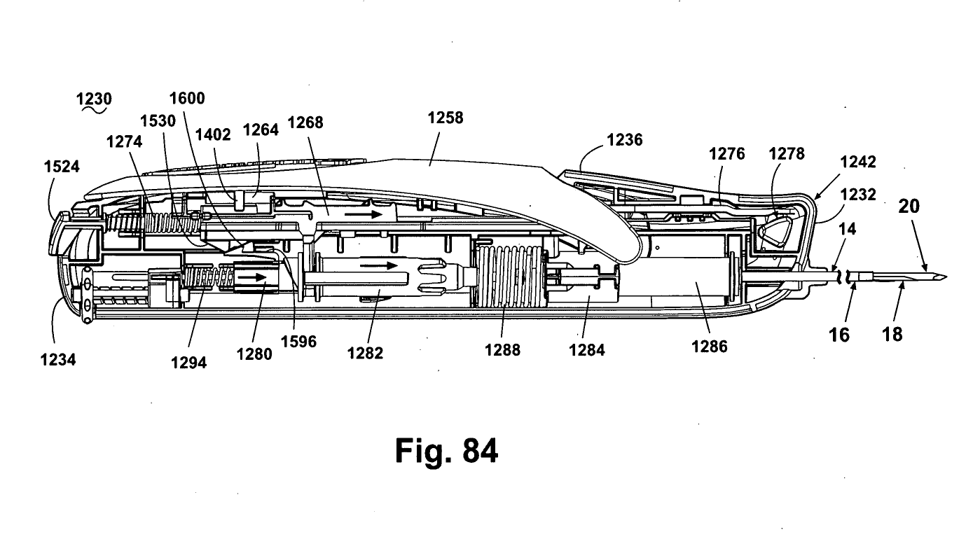

As illustrated in FIG. 84, the movement of the catch 1566 out of engagement with the catch retainer 1479 will release the shuttle 1268 for translation toward the distal end 1232 under the influence of the spring 1274. This will translate the coring cannula carriage 1282 distally, which will initiate the movement of the coring cannula 16 over the spoon cannula 18, with the excising finger 70 translating along the arcuate wall 46 of the spoon cannula 18.

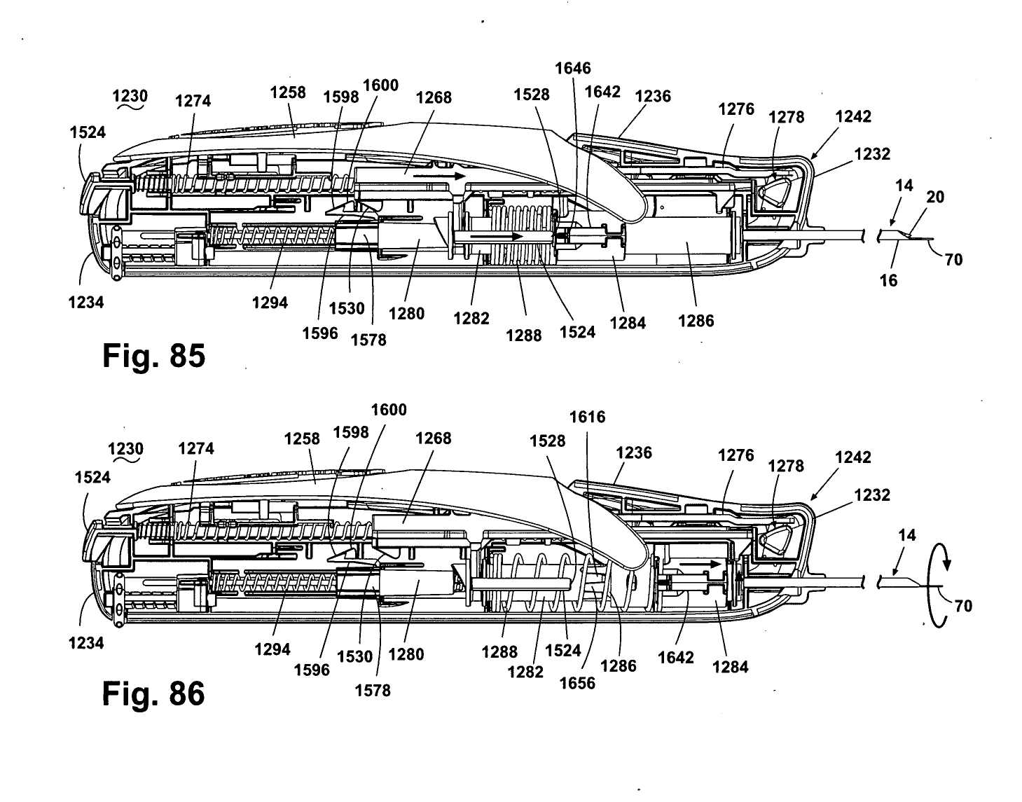

Referring to FIGS. 84 and 85, as the shuttle 1268 continues to translate toward the distal end 1232, the triangular boss 1530 will engage the fin 1600 extending dorsally from the arm 1596 of the spoon cannula carriage 1280. This will deflect the resilient arm 1596 ventrally, releasing the hooks 1598 from the hook retainers 1347 (FIG. 59), and enabling the spoon cannula carriage 1280 to move toward the distally under the influence of the spring 1294. The coring cannula carriage 1282 and the spoon cannula carriage 1280 will thus translate toward the distal end 1232, thereby urging the coring cannula 16 and the spoon cannula 18 into the lesion 24. The coring cannula carriage 1282 will be brought into engagement with the rotating driven member 1286 and thus prevented from further translation toward the distal end 1232. The spoon cannula carriage 1280 will bottom out as the wing portions 1578 engage the stops 1344, 1346 (FIG. 59) in the housing shells 1244, 1246. The coring cannula carriage 1282 will bottom out after the spoon cannula carriage 1280 bottoms out due to the engagement of the coring cannula carriage 1282 with the rotating driven member 1286, so that the excising finger 70 extends distally of the insertion tip 48 of the spoon cannula 18, as illustrated in FIG. 84.

As the shuttle 1268 continues to translate toward the distal end 1232, the lower rails 1524 will translate in the slots 1360, toward the viewing window 1366. The end portions 1526 of the lower rails 1524 will engage the resilient arms 1642 of the helical drive member 1284. The tapered tips 1528 will engage the hooks 1646, urging the radially-inward deflection of the resilient arms 1642. This will urge the hooks 1646 out of registry with the viewing window 1366 (FIG. 59), freeing the helical drive member 1284 to translate toward the distal end 1232 under the influence of the spring 1288.

Referring to FIG. 86, as the helical drive member 1284 translates, the rotating driven member 1286 will be urged into rotation by the rotational motion of the helical channelway 1636 acting on the boss 1664 (FIG. 71-72). Rotation of the rotating driven member 1286 will urge the rotation of the coring cannula carriage 1282 due to the interlocking of the toothed edge 1656 of the rotating driven member 1286 with the toothed edge 1616 of the coring cannula carriage 1282. This will urge the excising finger 70 in a rotational motion, excising the sample 26 from the lesion 24. The cannula assembly 14 can then be withdrawn from the tissue mass 22 with the biopsy sample 26 retained in the spoon section 42 of the spoon cannula 18.

Referring to FIG. 80, as the shuttle 1268 moves distally, the catch 1566 will move up one of the cam surfaces 1477, as indicated by arrow III. One of the spring arms 1560 will come into contact with one of the housing shells 1242, 1244 and will bias the toggle piece 1276 back toward the centerline of the shuttle 1268 as the catch 1566 translates along the outside of the guide rails 1474, as indication by arrow IV. On the shuttle 1268 moves far enough relative to the toggle piece 1276 for the catch 1566 to clear the guide rail 1474, the toggle piece will pivot back to the orientation corresponding to FIG. 77, and the catch 1566 will seat in the second pocket 1482, as indicated by arrow V. The biopsy gun 1230 is thus reset to its uncocked condition and is ready for cocking or arming.

The core biopsy device 10 described herein provides several distinct advantages over the prior are which increase the probability of obtaining a high-quality biopsy sample. The use of a sample size control assembly allows the user to select the specimen size to be collected. The use of a spoon-shaped biopsy sample support minimizes the disturbance and degradation of the sample which can occur with devices having enclosed sample chambers requiring the sample be ejected by a plunger, the stylet, or similar means. This avoids the necessity of obtaining a second sample if the first one proves to be unusable. Additionally, the use of the rotational cutting mechanism ensures that the sample is completely excised from the surrounding tissue mass, thereby minimizing the potential for disturbance or degradation of the sample when pulling the sample away from the tissue mass in order to sever it. This also minimizes the potential that the sample will separate from the tissue mass at a location within the sample itself rather than at its attachment to the tissue mass, thereby avoiding a sample volume which is inadequate for analysis. Finally, the core biopsy device 10 can be inserted, cocked, and actuated by an operator using one hand, thereby enabling the operator to concurrently operate an imaging device, such as an ultrasound wand, for positioning the cannula assembly 14, eliminating the need for an additional imaging technician. Thus, the core biopsy device 10 is easily inserted and triggered with one hand, providing very quick recovery of a biopsy sample, thereby enhancing the quality of the biopsy sample and minimizing discomfort to the patient. Furthering aiding in one-handed operation is the provision of a rear trigger button, a forward left side trigger button, and a forward right side trigger button, any of which the user can utilize to actuate the core biopsy device 10.

While the invention has been specifically described in connection with certain specific embodiments thereof, it is to be understood that this is by way of illustration and not of limitation. Aspects of the invention, for example the provision of three trigger buttons and their placement on the biopsy device, can be readily applied to other biopsy devices such as notch biopsy devices, non-rotational biopsy devices, non-core biopsy devices, etc. Reasonable variation and modification are possible within the scope of the forgoing disclosure and drawings without departing from the spirit of the invention, which is defined in the appended claims.

What is claimed is:

1. A biopsy device for the percutaneous removal of a specimen from a tissue mass, the biopsy device comprising:
a stylet having a distal tip;
a coring cannula defining a lumen for receiving the stylet and having an excising finger at a distal end of the coring cannula and operable between an inserting position and an excising position, where the distal tip of the stylet is spaced from the distal end of the coring cannula a predetermined distance;
an actuator operably coupled to the coring cannula to move the coring cannula relative to the stylet from the inserting position to the excising position and to rotate the coring cannula in the excising position; and
a specimen length adjuster operably coupled to the stylet to move the stylet relative to the coring cannula to set the predetermined distance.

2. The biopsy device according to claim 1, wherein the stylet is distally spaced the predetermined distance prior to firing the biopsy device.

3. The biopsy device according to claim 1 wherein the adjuster is discretely adjustable.

4. The biopsy device according to claim 3 wherein the adjuster comprises an adjustment member that is coupled to the stylet and is discretely positionable relative to the coring cannula to set the predetermined distance.

5. The biopsy device according to claim 4, and further comprising a housing, wherein a portion of the adjustment member protrudes exteriorly of the housing to position the adjustment member relative to the coring cannula to set the predetermined distance.

6. The biopsy device according to claim 1 wherein the adjuster is infinitely adjustable.

7. The biopsy device according to claim 6 wherein the adjuster comprises a adjustment member and a stylet carriage that carries the stylet and is movably mounted on the adjustment member such that movement of the adjustment member is translated to movement of the stylet carriage to set the predetermined distance.

8. The biopsy device according to claim 7 wherein rotational movement of the adjustment member is translated to linear movement of the stylet carriage to set the predetermined distance.

9. The biopsy device according to claim 1, and further comprising a handle that carries the stylet and the coring cannula, and contains the actuator and the specimen length adjuster.

10. The biopsy device according to claim 9 wherein the handle is a self-contained unit.

11. A method of conducting a percutaneous biopsy by removing a core specimen from a predetermined site in a tissue mass with a biopsy device comprising an axially movable coring cannula defining a lumen, and a stylet received within the lumen, the method comprising:
setting the length of the core specimen by adjusting the relative positions of the stylet and the coring cannula;
partially forming a core specimen by moving the coring cannula relative to the stylet; and
severing the core specimen from the tissue mass by rotating the coring cannula.

12. The method according to claim 11 wherein the severing step takes place after the partially forming step.

13. The method according to claim 11 and further comprising arming the biopsy device by moving the coring cannula relative to the stylet.

14. The method according to claim 13 wherein the setting step takes place after the arming step.

15. The method according to claim 11 wherein the setting step comprises discretely adjusting the relative positions of the stylet and the coring cannula.

16. The method according to claim 11 wherein the setting step comprises infinitely adjusting the relative positions of the stylet and the coring cannula.

17. A method of operating a biopsy device comprising an axially movable coring cannula defining a lumen, and a stylet received within the lumen, the method comprising:
axially moving the coring cannula relative to the stylet from a ready position to a cutting position to form a tissue chamber between a distal end of the stylet and a distal end of the coring cannula;
rotating the coring cannula in the cutting position; and
adjusting the relative positions of the stylet and the coring cannula to place the biopsy device in the ready position and to define the length of the tissue chamber.

18. The method according to claim 17 wherein the adjusting step comprises discretely adjusting the relative positions of the stylet and the coring cannula.

19. The method according to claim 17 wherein the adjusting step comprises infinitely adjusting the relative positions of the stylet and the coring cannula.

20. A biopsy device for the percutaneous removal of a specimen from a tissue mass, the biopsy device comprising:
a housing having opposing sides;
a needle assembly carried by the housing and comprising a stylet having a distal tip and a cannula defining a lumen for receiving the stylet;
a firing assembly carried by the housing and operably coupled to the needle assembly to effect the relative movement of the cannula and the stylet to obtain a specimen; and
a trigger assembly operably coupled to the firing assembly to control the actuation of the firing assembly and having a release button on each opposing side of the housing, wherein pressing either release button effects the actuation of the firing assembly.

21. The biopsy device according to claim 20 wherein the housing comprises a distal end and a proximal end, and the release buttons are located near the distal end of the housing.

22. The biopsy device according to claim 21 wherein the trigger assembly further comprises a rear release button located near the proximal end of the housing, wherein pressing the rear release button effects actuation of the firing assembly.

23. The biopsy device according to claim 20 wherein the housing has hollow interior and the firing assembly is located in the hollow interior.

24. The biopsy device according to claim 20 wherein the opposing sides of the housing comprise a left and right side.

25. The biopsy device according to claim 20 wherein a single member forms both release buttons.

26. The biopsy device according to claim 20, and further comprising a cocking assembly operably coupled to the firing assembly to arm the firing assembly prior to actuation of the firing assembly.

27. The biopsy device according to claim 26 wherein the cocking assembly comprises a catch and actuation of the cocking assembly moves the catch from a first position in which the firing assembly is unarmed to a second position in which the firing assembly is armed and the catch prevents actuation of the firing assembly.

28. The biopsy device according to claim 27 wherein pressing either release button effects movement of the catch from the second position to actuate the firing assembly.

29. The biopsy device according to claim 28 wherein the catch is configured to automatically move to the first position after the firing assembly is actuated.
